(19) United States
(12) Patent Application Publication
(10) Pub. No.: US 2009/0186766 A1
(43) Pub. Date: Jul. 23, 2009

(54) Heteroaroyl-Substituted Alanines with a Herbicidal Action

(75) Inventors: Matthias Witschel, Bad Dürkheim (DE); Cyrill Zagar, Hong Kong (CN); Eike Hupe, Ludwigshafen (DE); Toralf Kühn, Mannheim (DE); William Karl Moberg, Hassloch (DE); Liliana Parra Rapado, Offenburg (DE); Frank Stelzer, Mannheim (DE); Andrea Vescovi, Mannheim (DE); Michael Rack, Eppelheim (DE); Robert Reinhard, Limburgerhof (DE); Bernd Sievernich, Hassloch (DE); Klaus Grossmann, Neuhofen (DE); Thomas Ehrhardt, Speyer (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/300,884

(22) PCT Filed: May 11, 2007
(86) PCT No.: PCT/EP07/54562

§ 371 Date: Nov. 14, 2008

(30) Foreign Application Priority Data
May 19, 2006 (EP) 06114239.4

(51) Int. Cl. A01N 43/56; C07D 231/12; A01P 13/00

(52) U.S. Cl. 504/280; 548/374.1

(57) ABSTRACT

The present invention relates to heteroaroyl-substituted alanines of the formula I

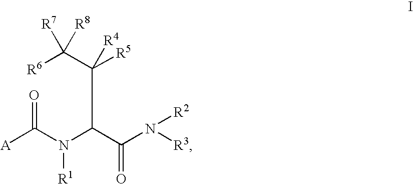

in which the variables A and R¹ to R⁸ are as defined in the description,
and to their agriculturally useful salts,
to processes and intermediates for their preparation, and to the use of these compounds or of compositions comprising these compounds for controlling unwanted plants.

The present invention relates to heteroaroyl-substituted alanines of the formula I

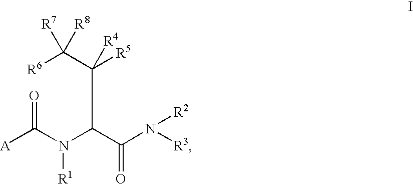

in which the variables are as defined below;
A is 5- or 6-membered heteroaryl having one to four nitrogen atoms, or having one to three nitrogen atoms and one oxygen or sulfur atom, or having one oxygen or sulfur atom, which heteroaryl may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
R¹, R² are hydrogen, hydroxyl or C₁-C₆-alkoxy;
R³ is C₁-C₆-alkyl, C₁-C₄-cyanoalkyl or C₁-C₆-haloalkyl;
R⁴ is hydrogen or C₁-C₆-alkyl;
R⁵ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₂-C₆-cyanoalkenyl, C₂-C₆-cyanoalkynyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, 3- to 6-membered heterocyclyl,
   where the cycloalkyl, cycloalkenyl or 3- to 6-membered heterocyclyl radicals mentioned above may be partially or fully halogenated and/or may carry one to three radicals from the group consisting of oxo, cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, aminocarbonylamino, (C₁-C₆-alkylamino)carbonylamino, di(C₁-C₆-alkyl)-aminocarbonylamino, aryl and aryl(C₁-C₆-alkyl);
C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₄-alkyl, C₁-C₆-haloalkyl-C₁-C₄-thioalkyl, C₂-C₆-haloalkenyl-C₁-C₄-thioalkyl, C₂-C₆-haloalkynyl-C₁-C₄-thioalkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, amino-C₁-C₄-alkyl, (C₁-C₆-alkyl)amino-C₁-C₄-alkyl, di(C₁-C₆-alkyl)amino-C₁-C₄-alkyl, (C₁-C₆-alkylsulfonyl)amino-C₁-C₄-alkyl, C₁-C₆-alkylsulfonyl(C₁-C₆-alkyl)amino-C₁-C₄-alkyl, C₁-C₆-alkylcarbonyl, hydroxycarbonyl, C₁-C₆-alkoxycarbonyl, aminocarbonyl, (C₁-C₆-alkyl)aminocarbonyl, di(C₁-C₆-alkyl)aminocarbonyl, formylamino-C₁-C₄-alkyl, (C₁-C₆-alkoxycarbonyl)amino-C₁-C₄-alkyl, C₁-C₆-alkylcarbonyl-C₁-C₆-alkyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, (C₁-C₆-alkyl)aminocarbonyl-C₁-C₄-alkyl, di(C₁-C₆-alkyl)aminocarbonyl-C₁-C₄-alkyl, (C₁-C₆-alkylcarbonyl)amino-C₁-C₄-alkyl, C₁-C₆-alkylcarbonyl(C₁-C₆-alkylamino)-C₁-C₄-alkyl, (C₁-C₆-alkyl)aminocarbonyloxy-C₁-C₄-alkyl, di(C₁-C₆-alkyl)aminocarbonyloxy-C₁-C₄-alkyl, (C₁-C₆-alkyl)aminocarbonylamino-C₁-C₄-alkyl, di(C₁-C₆-alkyl)aminocarbonylamino-C₁-C₄-alkyl;
phenyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl, phenyl-C₁-C₄-hydroxyalkyl, phenyl-C₂-C₄-hydroxyalkenyl, phenyl-C₂-C₄-hydroxyalkynyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl,
heteroaryl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl,
   where the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxyl, C₁-C₆-hydroxyalkyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, (C₁-C₆-alkyl)aminocarbonylamino, di(C₁-C₆-alkyl)-aminocarbonylamino, aryl and aryl(C₁-C₆-alkyl);
R⁶ is OR⁹, NR¹⁰R¹¹ or NO₂;
R⁷ is hydrogen, C₁-C₆-alkyl or C₁-C₆-haloalkyl;
R⁸ is hydrogen, C₁-C₆-alkyl or C₁-C₆-haloalkyl;
R⁹ and R¹⁰ are hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₁-C₆-alkylthiocarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, [(C₁-C₆-alkyl)aminocarbonyl(C₁-C₆-alkyl)amino]carbonyl, (C₁-C₆-alkyl)aminothiocarbonyl, di(C₁-C₆-alkyl)aminothiocarbonyl, (C₁-C₆-alkyl)cyanoimino, (amino)cyanoimino, (C₁-C₆-alkyl)aminocyanoimino, di(C₁-C₆-alkyl)aminocyanoimino, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-(di-C₁-C₆-alkylamino)imino-C₁-C₆-alkyl or tri-C₁-C₄-alkylsilyl,
   where the alkyl, cycloalkyl and alkoxy radicals mentioned may be partially or fully halogenated and/or may carry one to three of the following groups: cyano, hydroxyl, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl or C₁-C₄-alkylcarbonyloxy;
phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl,
   where the phenyl radical may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
SO₂R¹²;
R¹¹ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, hydroxy or C₁-C₆-alkoxy;
R¹² is C₁-C₆-alkyl, C₁-C₆-haloalkyl, di(C₁-C₆-alkyl)amino or phenyl,
   where the phenyl radical may be partially or fully halogenated and/or may carry one to three of the following groups: C₁-C₆-alkyl, C₁-C₆-haloalkyl or C₁-C₆-alkoxy;
      and their agriculturally useful salts.

Moreover, the invention relates to processes and intermediates for preparing compounds of the formula I, to compositions comprising them and to the use of these derivatives or of the compositions comprising them for controlling harmful plants.

2,ω-Diaminocarbonyl compounds with herbicidal activity are described, inter alia, in WO 03/045878.

Also known from the literature (for example WO 05/061464) are heteroaroyl-substituted phenylalanines which may carry an optionally substituted amino group in the β-position.

However, the herbicidal properties of the prior-art compounds and/or their compatibility with crop plants are not entirely satisfactory.

Accordingly, it is an object of the present invention to provide novel, in particular herbicidally active, compounds having improved properties.

We have found that this object is achieved by the heteroaroyl-substituted alanines of the formula I and their herbicidal action.

Furthermore, we have found herbicidal compositions which comprise the compounds I and have very good herbicidal action. Moreover, we have found processes for preparing these compositions and methods for controlling unwanted vegetation using the compounds I.

Depending on the substitution pattern, the compounds of the formula I comprise two or more centers of chirality, in which case they are present as enantiomers or diastereomer mixtures. The invention provides both the pure enantiomers or diastereomers and their mixtures.

The compounds of the formula I may also be present in the form of their agriculturally useful salts, the nature of the salt generally being immaterial. Suitable salts are, in general, the salts of those cations or the acid addition salts of those acids whose cations and anions, respectively, have no adverse effect on the herbicidal action of the compounds I.

Suitable cations are in particular ions of the alkali metals, preferably lithium, sodium and potassium, of the alkaline earth metals, preferably calcium and magnesium, and of the transition metals, preferably manganese, copper, zinc and iron, and also ammonium, where, if desired, one to four hydrogen atoms may be replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, dimethylammonium, diisopropylammonium, tetramethylammonium, tetrabutylammonium, 2-(2-hydroxyeth-1-oxy)eth-1-yl-ammonium, di-(2-hydroxyeth-1-yl)ammonium, trimethylbenzylammonium, furthermore phosphonium ions, sulfonium ions, preferably tri(C₁-C₄-alkyl)sulfonium, and sulfoxonium ions, preferably tri(C₁-C₄alkyl)sulfoxonium.

Anions of useful acid addition salts are primarily chloride, bromide, fluoride, hydrogensulfate, sulfate, dihydrogenphosphate, hydrogenphosphate, nitrate, bicarbonate, carbonate, hexafluorosilicate, hexafluorophosphate, benzoate, and the anions of C₁-C₄-alkanoic acids, preferably formate, acetate, propionate and butyrate.

The organic moieties mentioned for the substituents R¹-R¹⁶ or as radicals on phenyl, aryl, heteroaryl or heterocyclyl rings are collective terms for individual enumerations of the specific group members.

All hydrocarbon chains, i.e. all alkyl, alkenyl, alkynyl, haloalkyl, haloalkenyl, haloalkynyl, cyanoalkyl, cyanoalkenyl, cyanoalkynyl, hydroxyalkyl, hydroxyalkenyl, hydroxyalkynyl, alkoxy, haloalkoxy and alkylthio moieties may be straight-chain or branched.

Unless indicated otherwise, halogenated substituents preferably carry one to five identical or different halogen atoms. The term halogen denotes in each case fluorine, chlorine, bromine or iodine.

Examples of other meanings are:
C₁-C₄-alkyl and also the alkyl moieties of tri-C₁-C₄-alkylsilyl, C₁-C₆-alkoxycarbonyl-C₁-C₄-alkyl, C₁-C₄-alkylcarbonyloxy, C₁-C₄-alkyl-C₁-C₆-alkoxycarbonylamino, C₁-C₄-alkoxy-C₁-C₄-alkoxy-C₁-C₄-alkyl, C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₄-alkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, amino-C₁-C₄-alkyl, C₁-C₆-alkylamino-C₁-C₄-alkyl, di(C₁-C₆-alkyl)amino-C₁-C₄-alkyl, formylamino-C₁-C₄-alkyl, C₁-C₆-alkoxycarbonylamino-C₁-C₄-alkyl, C₁-C₆-alkylsulfonylamino-C₁-C₄-alkyl, C₁-C₆-alkylsulfonyl-(C₁-C₆-alkyl)amino-C₁-C₄-alkyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, (C₁-C₆-alkyl)aminocarbonyl-C₁-C₄-alkyl, di(C₁-C₆-alkyl)aminocarbonyl-C₁-C₄-alkyl, (C₁-C₆-alkyl)aminocarbonylamino-C₁-C₄-alkyl, di(C₁-C₆-alkyl)aminocarbonylamino-C₁-C₄-alkyl, C₁-C₆-alkylcarbonylamino-C₁-C₄-alkyl, C₁-C₆-alkylcarbonyl-(C₁-C₆-alkyl)amino)-C₁-C₄-alkyl, (C₁-C₆-alkylamino)carbonyloxy-C₁-C₄-alkyl, di(C₁-C₆-alkylamino)carbonyloxy-C₁-C₄-alkyl, phenyl-C₁-C₄-alkyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl, heteroaryl-C₁-C₄-alkyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl, and aryl(C₁-C₄-alkyl):
   for example methyl, ethyl, n-propyl, 1-methylethyl, n-butyl, 1-methylpropyl, 2-methylpropyl and 1,1-dimethylethyl;
C₁-C₆-alkyl and also the alkyl moieties of C₁-C₆-cyanoalkyl, C₁-C₆-alkylcarbonyl-C₁-C₆-alkyl, N—(C₃-C₆-alkynyl)-N—(C₁-C₆-alkyl)aminocarbonyl, N—(C₁-C₆-alkoxy)-N—(C₁-C₆-alkyl)aminocarbonyl, C₁-C₈-alkylcarbonyl-C₁-C₆-alkyl, C₁-C₆-alkoxyimino-C₁-C₆-alkyl, N—(C₁-C₆-alkylamino)imino-C₁-C₆-alkyl, N-(di-C₁-C₆-alkylamino)imino-C₁-C₆-alkyl, (C₁-C₆-alkyl)cyanoimino, phenyl-C₁-C₆-alkyl, phenylcarbonyl-C₁-C₆-alkyl, N—(C₁-C₆-alkyl)-N-phenylaminocarbonyl:
C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1-ethyl-1-methylpropyl and 1-ethyl-3-methylpropyl;
C₁-C₄-alkylcarbonyl: for example methylcarbonyl, ethylcarbonyl, propylcarbonyl, 1-methylethylcarbonyl, butylcarbonyl, 1-methylpropylcarbonyl, 2-methylpropylcarbonyl or 1,1-dimethylethylcarbonyl;
C₁-C₆-alkylcarbonyl and also the alkylcarbonyl radicals of C₁-C₆-alkylcarbonyl-C₁-C₆-alkyl, C₁-C₆-alkylcarbonyloxy-C₁-C₈-alkyl, C₁-C₆-alkylcarbonylamino-C₁-C₄-alkyl, phenyl-C₁-C₆-alkylcarbonyl, C₁-C₆-alkylcarbonyl-(C₁-C₆-alkylamino)-C₁-C₄-alkyl: C₁-C₄-alkylcarbonyl as mentioned above, and also, for example, pentylcarbonyl, 1-methylbutylcarbonyl, 2-methylbutylcarbonyl, 3-methylbutylcarbonyl, 2,2-dimethylpropylcarbonyl, 1-ethylpropylcarbonyl, hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl;
C₃-C₆-cycloalkyl and also the cycloalkyl moieties of C₃-C₆-cycloalkylcarbonyl: monocyclic saturated hydrocarbons having 3 to 6 ring members, such as cyclopropyl, cyclobutyl, cyclopentyl and cyclohexyl;
C₃-C₆-cycloalkenyl: for example 1-cyclopropenyl, 2-cyclopropenyl, 1-cyclobutenyl, 2-cyclobutenyl, 1-cyclopentenyl, 2-cyclopentenyl, 1,3-cyclopentadienyl, 1,4-cyclopentadienyl, 2,4-cyclopentadienyl, 1-cyclohexenyl, 2-cyclohexenyl, 3-cyclohexenyl, 1,3-cyclohexadienyl, 1,4-cyclohexadienyl, 2,5-cyclohexadienyl;
C₃-C₆-alkenyl and also the alkenyl moieties of C₃-C₆-alkenyloxycarbonyl, C₃-C₆-alkenylaminocarbonyl, N—(C₃-C₆-alkenyl)-N—(C₁-C₆-alkyl)aminocarbonyl and N—(C₃-C₆-alkenyl)-N—(C₁-C₆-alkoxy)aminocarbonyl: for example 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
C₂-C₆-alkenyl and also the alkenyl moieties of C₂-C₆-alkenylcarbonyl, C₂-C₆-alkenyloxy-C₁-C₄-alkyl, C₂-C₆-alkenylthio-C₁-C₄-alkyl, phenyl-C₂-C₄-alkenyl, heteroaryl-C₂-C₄-alkenyl: C₃-C₆-alkenyl as mentioned above, and also ethenyl;
C₃-C₆-alkynyl and also the alkynyl moieties of C₃-C₆-alkynyloxycarbonyl, C₃-C₆-alkynylaminocarbonyl, N—(C₃-C₆-alkynyl)-N—(C₁-C₆-alkyl)aminocarbonyl, N—(C₃-C₆-alkynyl)-N—(C₁-C₆-alkoxy)aminocarbonyl: for example 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
C₂-C₆-alkynyl and also the alkynyl moieties of C₂-C₆-alkynylcarbonyl, C₂-C₂-alkynyloxy-C₁-C₄-alkyl, C₂-C₆-alkynylthio-C₁-C₄-alkyl, phenyl-C₂-C₄-alkynyl, heteroaryl-C₂-C₄-alkynyl: C₃-C₆-alkynyl as mentioned above, and also ethynyl;
C₁-C₄-cyanoalkyl: for example cyanomethyl, 1-cyanoeth-1-yl, 2-cyanoeth-1-yl, 1-cyanoprop-1-yl, 2-cyanoprop-1-yl, 3-cyanoprop-1-yl, 1-cyanoprop-2-yl, 2-cyanoprop-2-yl, 1-cyanobut-1-yl, 2-cyanobut-1-yl, 3-cyanobut-1-yl, 4-cyanobut-1-yl, 1-cyanobut-2-yl, 2-cyanobut-2-yl, 1-cyanobut-3-yl, 2-cyanobut-3-yl, 1-cyano-2-methylprop-3-yl, 2-cyano-2-methylprop-3-yl, 3-cyano-2-methylprop-3-yl and 2-cyanomethylprop-2-yl;
C₁-C₄-hydroxyalkyl and also the C₁-C₄-hydroxyalkyl moieties of phenyl-C₁-C₄-hydroxyalkyl, heteroaryl-C₁-C₄-hydroxyalkyl: for example hydroxymethyl, 1-hydroxyeth-1-yl, 2-hydroxyeth-1-yl, 1-hydroxyprop-1-yl, 2-hydroxyprop-1-yl, 3-hydroxyprop-1-yl, 1-hydroxyprop-2-yl, 2-hydroxyprop-2-yl, 1-hydroxybut-1-yl, 2-hydroxybut-1-yl, 3-hydroxybut-1-yl, 4-hydroxybut-1-yl, 1-hydroxybut-2-yl, 2-hydroxybut-2-yl, 1-hydroxybut-3-yl, 2-hydroxybut-3-yl, 1-hydroxy-2-methylprop-3-yl, 2-hydroxy-2-methylprop-3-yl, 3-hydroxy-2-methylprop-3-yl and 2-hydroxymethylprop-2-yl, 1,2-dihydroxyethyl, 1,2-dihydroxyprop-3-yl, 2,3-dihydroxyprop-3-yl, 1,2-dihydroxyprop-2-yl, 1,2-dihydroxybut-4-yl, 2,3-dihydroxybut-4-yl, 3,4-dihydroxybut-4-yl, 1,2-dihydroxybut-2-yl, 1,2-dihydroxybut-3-yl, 2,3-dihydroxybut-3-yl, 1,2-dihydroxy-2-methylprop-3-yl, 2,3-dihydroxy-2-methylprop-3-yl;
C₁-C₆-hydroxyalkyl: C₁-C₄-hydroxyalkyl as mentioned above and also, for example, 1-hydroxypent-5-yl, 2-hydroxypent-5-yl, 3-hydroxypent-5-yl, 4-hydroxypent-5-yl, 5-hydroxypent-5-yl, 1-hydroxypent-4-yl, 2-hydroxypent-4-yl, 3-hydroxypent-4-yl, 4-hydroxypent-4-yl, 1-hydroxypent-3-yl, 2-hydroxypent-3-yl, 3-hydroxypent-3-yl, 1-hydroxy-2-methylbut-3-yl, 2-hydroxy-2-methylbut-3-yl, 3-hydroxy-2-methylbut-3-yl, 1-hydroxy-2-methylbut-4-yl, 2-hydroxy-2-methylbut-4-yl, 3-hydroxy-2-methylbut-4-yl, 4-hydroxy-2-methylbut-4-yl, 1-hydroxy-3-methylbut-4-yl, 2-hydroxy-3-methylbut-4-yl, 3-hydroxy-3-methylbut-4-yl, 4-hydroxy-3-methylbut-4-yl, 1-hydroxyhex-6-yl, 2-hydroxyhex-6-yl, 3-hydroxyhex-6-yl, 4-hydroxyhex-6-yl, 5-hydroxyhex-6-yl, 6-hydroxyhex-6-yl, 1-hydroxy-2-methylpent-5-yl, 2-hydroxy-2-methylpent-5-yl, 3-hydroxy-2-methylpent-5-yl, 4-hydroxy-2-methylpent-5-yl, 5-hydroxy-2-methylpent-5-yl, 1-hydroxy-3-methylpent-5-yl, 2-hydroxy-3-methylpent-5-yl, 3-hydroxy-3-methylpent-5-yl, 4-hydroxy-3-methylpent-5-yl, 5-hydroxy-3-methylpent-5-yl, 1-hydroxy-4-methylpent-5-yl, 2-hydroxy-4-methylpent-5-yl, 3-hydroxy-4-methylpent-5-yl, 4-hydroxy-4-methylpent-5-yl, 5-hydroxy-4-methylpent-5-yl, 1-hydroxy-5-methylpent-5-yl, 2-hydroxy-5-methylpent-5-yl, 3-hydroxy-5-methylpent-5-yl, 4-hydroxy-5-methylpent-5-yl, 5-hydroxy-5-methylpent-5-yl, 1-hydroxy-2,3-dimethylbut-4-yl, 2-hydroxy-2,3-dimethylbut-4-yl, 3-hydroxy-2,3-dimethylbut-4-yl, 4-hydroxy-2,3-dimethylbut-4-yl, 1,2-dihydroxy-pent-5-yl, 2,3-dihydroxy-pent-5-yl, 3,4-dihydroxy-pent-5-yl, 4,5-dihydroxy-pent-5-yl, 1,2-dihydroxypent-4-yl, 2,3-dihydroxypent-4-yl, 3,4-dihydroxypent-4-yl, 4,5-dihydroxypent-4-yl, 1,2-dihydroxypent-3-yl, 2,3-dihydroxypent-3-yl, 1,2-dihydroxy-2-methylbut-3-yl, 2,3-dihydroxy-2-methylbut-3-yl, 3,4-dihydroxy-2-methylbut-3-yl, 2-hydroxy-2-hydroxymethylbut-3-yl, 1,2-dihydroxy-2-methylbut-4-yl, 2,3-dihydroxy-2-methylbut-4-yl, 3,4-dihydroxy-2-methylbut-4-yl, 1,2-dihydroxy-3-methylbut-4-yl, 2,3-dihydroxy-3-methylbut-4-yl, 3,4-dihydroxy-3-methylbut-4-yl, 3-hydroxy-3-hydroxymethylbut-4-yl, 1,2-dihydroxyhex-6-yl, 2,3-dihydroxy-hex-6-yl, 3,4-dihydroxyhex-6-yl, 4,5-dihydroxyhex-6-yl, 5,6-dihydroxyhex-6-yl, 1,2-dihydroxy-2-methylpent-5-yl, 2,3-dihydroxy-2-methylpent-5-yl, 3,4-dihydroxy-2-methylpent-5-yl, 4,5-dihydroxy-2-methylpent-5-yl, 2-hydroxy-2-hydroxymethylpent-5-yl, 1,2-dihydroxy-3-methylpent-5-yl, 2,3-dihydroxy-3-methylpent-5-yl, 3,4-dihydroxy-3-methylpent-5-yl, 4,5-dihydroxy-3-methylpent-5-yl, 3-hydroxy-3-hydroxymethylpent-5-yl, 1,2-dihydroxy-4-methylpent-5-yl, 2,3-dihydroxy-4-methylpent-5-yl, 3,4-dihydroxy-4-methylpent-5-yl, 4,5-dihydroxy-4-methylpent-5-yl, 4-hydroxy-4-hydroxymethylpent-5-yl, 1,2-dihydroxy-5-methylpent-5-yl, 2,3-dihydroxy-5-methylpent-5-yl, 3,4-dihydroxy-5-methylpent-5-yl, 4,5-dihydroxy-5-methylpent-5-yl, 5-hydroxy-5-hydroxymethylpent-5-yl, 1,2-dihydroxy-2,3-dimethylbut-4-yl, 2,3-dihydroxy-2,3-dimethylbut-4-yl, 3,4-dihydroxy-2,3-dimethylbut-4-yl, 2-hydroxy-2-hydroxymethyl-3-methylbut-4-yl, 3-hydroxy-3-hydroxymethyl-2-methylbut-4-yl;
C₁-C₄-haloalkyl and also the haloalkyl moieties of phenyl-C₁-C₄-haloalkyl, heteroaryl-C₁-C₄-haloalkyl: a C₁-C₄-alkyl radical as mentioned above which is partially or fully substituted by fluorine, chlorine, bromine and/or iodine, i.e., for example, chloromethyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl, nonafluorobutyl, 1,1,2,2-tetrafluoroethyl and 1-trifluoromethyl-1,2,2,2,2-tetrafluoroethyl;
C₁-C₆-haloalkyl and also the haloalkyl moieties of C₁-C₆-haloalkyl-C₁-C₄-thioalkyl: C₁-C₄-haloalkyl as mentioned above, and also, for example, 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl and tridecafluorohexyl;
C₃-C₆-haloalkenyl: a C₃-C₆-alkenyl radical as mentioned above which is partially or fully substituted by fluorine, chlorine, bromine and/or iodine, for example 2-chloroprop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl or 2,3-dibromobut-2-en-1-yl;
C₂-C₆-haloalkenyl and also the C₂-C₆-haloalkenyl moieties of C₂-C₆-haloalkenyloxy-C₁-C₄-alkyl, C₂-C₆-haloalkenyl-C₁-C₄-thioalkyl, phenyl-C₂-C₄-haloalkenyl, heteroaryl-C₂-C₄-haloalkenyl: a C₂-C₆-alkenyl radical as mentioned above which is partially or fully substituted by fluorine, chlorine, bromine and/or iodine: for example 2-chlorovinyl, 2-chloroallyl, 3-chloroallyl, 2,3-dichloroallyl, 3,3-dichloroallyl, 2,3,3-trichloroallyl, 2,3-dichlorobut-2-enyl, 2-bromovinyl, 2-bromoallyl, 3-bromoallyl, 2,3-dibromoallyl, 3,3-dibromoallyl, 2,3,3-tribromoallyl or 2,3-dibromobut-2-enyl;
C₂-C₆-cyanoalkenyl: for example 2-cyanovinyl, 2-cyanoallyl, 3-cyanoallyl, 2,3-dicyanoallyl, 3,3-dicyanoallyl, 2,3,3-tricyanoallyl, 2,3-dicyanobut-2-enyl;
C₂-C₆-hydroxyalkenyl and also the hydroxyl moieties of phenyl-C₁-C₄-hydroxyalkenyl, heteroaryl-C₁-C₄-hydroxyalkenyl: for example 2-hydroxyvinyl, 2-hydroxyallyl, 3-hydroxyallyl, 2,3-dihydroxyallyl, 3,3-dihydroxyallyl, 2,3,3-trihydroxyallyl, 2,3-dihydroxybut-2-enyl;
C₃-C₆-haloalkynyl: a C₃-C₆-alkynyl radical as mentioned above which is partially or fully substituted by fluorine, chlorine, bromine and/or iodine, for example 1,1-difluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
C₂-C₆-haloalkynyl and also the C₂-C₆-haloalkynyl moieties of C₂-C₆-haloalkynyloxy-C₁-C₄-alkyl, C₂-C₆-haloalkynyl-C₁-C₄-thioalkyl, phenyl-C₂-C₄-haloalkynyl, heteroaryl-C₂-C₄-haloalkynyl: a C₂-C₆-alkynyl radical as mentioned above which is partially or fully substituted by fluorine, chlorine, bromine and/or iodine, for example 1,1-difluoroprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl or 6-iodohex-5-yn-1-yl;
C₂-C₆-cyanoalkynyl: for example 1,1-dicyanoprop-2-yn-1-yl, 3-cyanoprop-2-yn-1-yl, 4-cyano-but-2-yn-1-yl, 1,1-dicyanobut-2-yn-1-yl, 4-cyanobut-3-yn-1-yl, 5-cyanopent-3-yn-1-yl, 5-cyanopent-4-yn-1-yl, 6-cyanohex-4-yn-1-yl or 6-cyanohex-5-yn-1-yl;
C₂-C₆-hydroxyalkynyl and also the hydroxyl moieties of phenyl-C₂-C₄-hydroxyalkynyl, heteroaryl-C₂-C₄-hydroxyalkynyl: for example 1,1-dihydroxyprop-2-yn-1-yl, 3-hydroxyprop-2-yn-1-yl, 4-hydroxybut-2-yn-1-yl, 1,1-dihydroxybut-2-yn-1-yl, 4-hydroxybut-3-yn-1-yl, 5-hydroxypent-3-yn-1-yl, 5-hydroxypent-4-yn-1-yl, 6-hydroxyhex-4-yn-1-yl or 6-hydroxyhex-5-yn-1-yl;
C₁-C₆-alkylsulfinyl (C₁-C₆-alkyl-S(═O)—) and also the C₁-C₆-alkylsulfinyl moieties of C₁-C₆-alkylsulfinyl-C₁-C₄-alkyl: for example methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl;
C₁-C₆-haloalkylsulfinyl and also the C₁-C₆-haloalkylsulfinyl moieties of C₁-C₆-haloalkylsulfinyl-C₁-C₄-alkyl: C₁-C₆-alkylsulfinyl radical as mentioned above which is partially or fully substituted by fluorine, chlorine, bromine and/or iodine, i.e. for example fluoromethylsulfinyl, difluoromethylsulfinyl, trifluoromethylsulfinyl, chlorodifluoromethylsulfinyl, bromodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2,2,2-trichloroethylsulfinyl, 2-chloro-2-fluoroethylsulfinyl, 2-chloro-2,2-difluoroethylsulfinyl, 2,2-dichloro-2-fluoroethylsulfinyl, pentafluoroethylsulfinyl, 2-fluoropropylsulfinyl, 3-fluoropropylsulfinyl, 2-chloropropylsulfinyl, 3-chloropropylsulfinyl, 2-bromopropylsulfinyl, 3-bromopropylsulfinyl, 2,2-difluoropropylsulfinyl, 2,3-difluoropropylsulfinyl, 2,3-dichloropropylsulfinyl, 3,3,3-trifluoropropylsulfinyl, 3,3,3-trichloropropylsulfinyl, 2,2,3,3,3-pentafluoropropylsulfinyl, heptafluoropropylsulfinyl, 1-(fluoromethyl)-2-fluoroethylsulfinyl, 1-(chloromethyl)-2-chloroethylsulfinyl, 1-(bromomethyl)-2-bromoethylsulfinyl, 4-fluorobutylsulfinyl, 4-chlorobutylsulfinyl, 4-bromobutylsulfinyl, nonafluorobutylsulfinyl, 5-fluoropentylsulfinyl, 5-chloropentylsulfinyl, 5-bromopentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl and dodecafluorohexylsulfinyl;
C₁-C₆-alkylsulfonyl (C₁-C₆-alkyl-S(O)₂—) and also the C₁-C₆-alkylsulfonyl moieties of C₁-C₆-alkylsulfonyl-C₁-C₄-alkyl, C₁-C₆-alkylsulfonylamino, C₁-C₆-alkylsulfonylamino-C₁-C₄-alkyl, C₁-C₆-alkylsulfonyl-(C₁-C₆-alkyl)amino-C₁-C₄-alkyl, C₁-C₆-alkylsulfonylaminocarbonyl: for example methylsulfonyl, ethylsulfonyl, propylsulfonyl, 1-methylethylsulfonyl, butylsulfonyl, 1-methylpropylsulfonyl, 2-methylpropylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl;
C₁-C₆-haloalkylsulfonyl and also the C₁-C₆-haloalkylsulfonyl moieties of C₁-C₆-haloalkylsulfonyl-C₁-C₄-alkyl, C₁-C₆-haloalkylsulfonylamino: a C₁-C₆-alkylsulfonyl radical as mentioned above which is partially or fully substituted by fluorine, chlorine, bromine and/or iodine, i.e. for example fluoromethylsulfonyl, difluoromethylsulfonyl, trifluoromethylsulfonyl, chlorodifluoromethylsulfonyl, bromodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, pentafluoroethylsulfonyl, 2-fluoropropylsulfonyl, 3-fluoropropylsulfonyl, 2-chloropropylsulfonyl, 3-chloropropylsulfonyl, 2-bromopropylsulfonyl, 3-bromopropylsulfonyl, 2,2-difluoropropylsulfonyl, 2,3-difluoropropylsulfonyl, 2,3-dichloropropylsulfonyl, 3,3,3-trifluoropropylsulfonyl, 3,3,3-trichloropropylsulfonyl, 2,2,3,3,3-pentafluoropropylsulfonyl, heptafluoropropylsulfonyl, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl, nonafluorobutylsulfonyl, 5-fluoropentylsulfonyl, 5-chloropentylsulfonyl, 5-bromopentylsulfonyl, 5-iodopentylsulfonyl, 6-fluorohexylsulfonyl, 6-bromohexylsulfonyl, 6-iodohexylsulfonyl and dodecafluorohexylsulfonyl;
C₁-C₄-alkoxy and also the alkoxy moieties of hydroxycarbonyl-C₁-C₄-alkoxy, C₁-C₄-alkoxycarbonyl-C₁-C₄-alkoxy, C₁-C₄-alkoxy-C₁-C₄-alkoxy-C₁-C₄-alkyl and C₁-C₄-alkyl-C₁-C₄-alkoxycarbonylamino: for example methoxy, ethoxy, propoxy, 1-methylethoxy, butoxy, 1-methylpropoxy, 2-methylpropoxy and 1,1-dimethylethoxy;
C₁-C₆-alkoxy and also the alkoxy moieties of hydroxycarbonyl-C₁-C₆-alkoxy, C₁-C₄-alkyl-C₁-C₆-alkoxycarbonylamino, C₁-C₆-alkoxycarbonyl-C₁-C₆-alkoxy, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, C₁-C₆-alkoxyimino-C₁-C₆-alkyl and C₁-C₆-alkoxy-C₁-C₄-alkoxy: C₁-C₄-alkoxy as mentioned above, and also, for example, pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;
C₁-C₄-haloalkoxy: a C₁-C₄-alkoxy radical as mentioned above which is partially or fully substituted by fluorine, chlorine, bromine and/or iodine, i.e., for example, fluoromethoxy, difluoromethoxy, trifluoromethoxy, chlorodifluoromethoxy, bromodifluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy and nonafluorobutoxy;
C₁-C₆-haloalkoxy and also the C₁-C₆-haloalkoxy moieties of C₁-C₆-haloalkoxy-C₁-C₄-alkyl, C₁-C₆-haloalkoxycarbonyl-C₁-C₄-alkyl: C₁-C₄-haloalkoxy as mentioned above, and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy and dodecafluorohexoxy;
C₁-C₆-alkoxy-C₁-C₄-alkyl and also the C₁-C₆-alkoxy-C₁-C₄-alkyl moieties of C₁-C₆-alkoxy-C₁-C₄-alkoxy-C₁-C₄-alkyl: C₁-C₄-alkyl which is substituted by C₁-C₆-alkoxy as mentioned above, i.e., for example, methoxymethyl, ethoxymethyl, propoxymethyl, (1-methylethoxy)methyl, butoxymethyl, (1-methylpropoxy)methyl, (2-methylpropoxy)methyl, (1,1-dimethylethoxy)methyl, 2-(methoxy)ethyl, 2-(ethoxy)ethyl, 2-(propoxy)ethyl, 2-(1-methylethoxy)ethyl, 2-(butoxy)ethyl, 2-(1-methylpropoxy)-ethyl, 2-(2-methylpropoxy)ethyl, 2-(1,1-dimethylethoxy)ethyl, 2-(methoxy)propyl, 2-(ethoxy)propyl, 2-(propoxy)propyl, 2-(1-methylethoxy)propyl, 2-(butoxy)propyl, 2-(1-methylpropoxy)propyl, 2-(2-methylpropoxy)propyl, 2-(1,1-dimethylethoxy)-propyl, 3-(methoxy)propyl, 3-(ethoxy)propyl, 3-(propoxy)propyl, 3-(1-methylethoxy)-propyl, 3-(butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(propoxy)butyl, 2-(1-methylethoxy)butyl, 2-(butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(propoxy)butyl, 3-(1-methylethoxy)butyl, 3-(butoxy)butyl, 3-(1-methylpropoxy)-butyl, 3-(2-methylpropoxy)butyl, 3-(1,1-dimethylethoxy)butyl, 4-(methoxy)butyl, 4-(ethoxy)butyl, 4-(propoxy)butyl, 4-(1-methylethoxy)butyl, 4-(butoxy)butyl, 4-(1-methylpropoxy)butyl, 4-(2-methylpropoxy)butyl and 4-(1,1-dimethylethoxy)butyl;
C₁-C₄-alkoxycarbonyl and also the alkoxycarbonyl moieties of C₁-C₄-alkoxycarbonyl-C₁-C₄-alkoxy, C₁-C₄-alkoxy-C₁-C₄-alkoxycarbonyl and di-(C₁-C₄-alkyl)amino-C₁-C₄-alkoxycarbonyl: for example methoxycarbonyl, ethoxycarbonyl, propoxycarbonyl, 1-methylethoxycarbonyl, butoxycarbonyl, 1-methylpropoxycarbonyl, 2-methylpropoxycarbonyl or 1,1-dimethylethoxycarbonyl;
C₁-C₆-alkoxycarbonyl and also the alkoxycarbonyl moieties of C₁-C₆-alkoxycarbonyl-C₁-C₆-alkoxy, (C₁-C₆-alkoxycarbonyl)amino-C₁-C₄-alkyl, C₁-C₆-alkoxycarbonyl-C₁-C₄-alkyl, C₁-C₄-alkyl-(C₁-C₆-alkoxycarbonyl)amino: C₁-C₄-alkoxycarbonyl as mentioned above, and also, for example, pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl;
C₁-C₄-alkylthio and also the C₁-C₄-alkylthio moieties of C₁-C₆-haloalkyl-C₁-C₄-thioalkyl, C₂-C₆-haloalkenyl-C₁-C₄-thioalkyl, C₂-C₆-haloalkynyl-C₁-C₄-thioalkyl: for example methylthio, ethylthio, propylthio, 1-methylethylthio, butylthio, 1-methylpropylthio, 2-methylpropylthio and 1,1-dimethylethylthio;
C₁-C₆-alkylthio and also the C₁-C₆-alkylthio moieties of C₁-C₆-alkylthio-C₁-C₄-alkyl: C₁-C₄-alkylthio as mentioned above, and also, for example, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutyl-thio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;
C₁-C₆-alkylamino and also the C₁-C₆-alkylamino radicals of N—(C₁-C₆-alkylamino)imino-C₁-C₆-alkyl, (C₁-C₆-alkyl)amino-C₁-C₄-alkyl, C₁-C₆-alkylsulfonyl-(C₁-C₆-alkyl)amino-C₁-C₄-alkyl, C₁-C₆-alkylcarbonyl-(C₁-C₆-alkylamino)-C₁-C₄-alkyl, (C₁-C₆-alkyl)aminothiocarbonyl and (C₁-C₆-alkyl)aminocyanoimino: for example methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino;
di(C₁-C₄-alkyl)amino: for example N,N-dimethylamino, N,N-diethylamino, N,N-dipropylamino, N,N-di-(1-methylethyl)amino, N,N-dibutylamino, N,N-di-(1-methylpropyl)amino, N,N-di-(2-methylpropyl)amino, N,N-di-(1,1-dimethylethyl)amino, N-ethyl-N-methylamino, N-methyl-N-propylamino, N-methyl-N-(1-methylethyl)amino, N-butyl-N-methylamino, N-methyl-N-(1-methylpropyl)-amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino and N-(1,1-dimethylethyl)-N-(2-methylpropyl)amino;
di(C₁-C₆-alkyl)amino and also the dialkylamino radicals of N-(di-C₁-C₆-alkylamino)-imino-C₁-C₆-alkyl, di(C₁-C₆-alkyl)amino-C₁-C₄-alkyl and di(C₁-C₆-alkyl)aminocyanoimino: di(C₁-C₄-alkyl)amino as mentioned above, and also, for example, N,N-dipentylamino, N,N-dihexylamino, N-methyl-N-pentylamino, N-ethyl-N-pentylamino, N-methyl-N-hexylamino and N-ethyl-N-hexylamino; (C₁-C₄-alkylamino)carbonyl: for example methylaminocarbonyl, ethylaminocarbonyl, propylaminocarbonyl, 1-methylethylaminocarbonyl, butylaminocarbonyl, 1-methylpropylaminocarbonyl, 2-methylpropylaminocarbonyl or 1,1-dimethylethylaminocarbonyl;
(C₁-C₄-alkyl)aminocarbonyl and also the (C₁-C₄-alkyl)aminocarbonyl moieties of (C₁-C₄-alkylamino)carbonylamino: for example methylaminocarbonyl, ethylaminocarbonyl, propylaminocarbonyl, 1-methylethylaminocarbonyl, butylaminocarbonyl, 1-methylpropylaminocarbonyl, 2-methylpropylaminocarbonyl or 1,1-dimethylethylaminocarbonyl;
di(C₁-C₄-alkyl)aminocarbonyl and also the di(C₁-C₄-alkyl)aminocarbonyl moieties of di(C₁-C₄-alkyl)aminocarbonylamino: for example N,N-dimethylaminocarbonyl, N,N-diethylaminocarbonyl, N,N-di-(1-methylethyl)aminocarbonyl, N,N-dipropylaminocarbonyl, N,N-dibutylaminocarbonyl, N,N-di-(1-methylpropyl)aminocarbonyl, N,N-di-(2-methylpropyl)aminocarbonyl, N,N-di-(1,1-dimethylethyl)aminocarbonyl, N-ethyl-N-methylaminocarbonyl, N-methyl-N-propylaminocarbonyl, N-methyl-N-(1-methylethyl)aminocarbonyl, N-butyl-N-methylaminocarbonyl, N-methyl-N-(1-methylpropyl)aminocarbonyl, N-methyl-N-(2-methylpropyl)aminocarbonyl, N-(1,1-dimethylethyl)-N-methylaminocarbonyl, N-ethyl-N-propylaminocarbonyl, N-ethyl-N-(1-methylethyl)aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N-(1,1-dimethylethyl)aminocarbonyl, N-(1-methylethyl)-N-propylaminocarbonyl, N-butyl-N-propylaminocarbonyl, N-(1-methylpropyl)-N-propylaminocarbonyl, N-(2-methylpropyl)-N-propylaminocarbonyl, N-(1,1-dimethylethyl)-N-propylaminocarbonyl, N-butyl-N-(1-methylethyl)aminocarbonyl, N-(1-methylethyl)-N-(1-methylpropyl)aminocarbonyl, N-(1-methylethyl)-N-(2-methylpropyl)aminocarbonyl, N-(1,1-dimethylethyl)-N-(1-methylethyl)aminocarbonyl, N-butyl-N-(1-methylpropyl)aminocarbonyl, N-butyl-N-(2-methylpropyl)aminocarbonyl, N-butyl-N-(1,1-dimethylethyl)aminocarbonyl, N-(1-methylpropyl)-N-(2-methylpropyl)aminocarbonyl, N-(1,1-dimethylethyl)-N-(1-methylpropyl)aminocarbonyl or N-(1,1-dimethylethyl)-N-(2-methylpropyl)aminocarbonyl;
(C₁-C₆-alkyl)aminocarbonyl and also the (C₁-C₆-alkyl)aminocarbonyl moieties of (C₁-C₆-alkylamino)carbonylamino, (C₁-C₆-alkyl)aminocarbonyl-C₁-C₄-alkyl, (C₁-C₆-alkyl)aminocarbonyloxy-C₁-C₄-alkyl and (C₁-C₆-alkyl)aminocarbonylamino-C₁-C₄-alkyl: (C₁-C₄-alkylamino)carbonyl as mentioned above, and also, for example, pentylaminocarbonyl, 1-methylbutylaminocarbonyl, 2-methylbutylaminocarbonyl, 3-methylbutylaminocarbonyl, 2,2-dimethylpropylaminocarbonyl, 1-ethylpropylaminocarbonyl, hexylaminocarbonyl, 1,1-dimethylpropylaminocarbonyl, 1,2-dimethylpropylaminocarbonyl, 1-methylpentylaminocarbonyl, 2-methylpentylaminocarbonyl, 3-methylpentylaminocarbonyl, 4-methylpentylaminocarbonyl, 1,1-dimethylbutylaminocarbonyl, 1,2-dimethylbutylaminocarbonyl, 1,3-dimethylbutylaminocarbonyl, 2,2-dimethylbutylaminocarbonyl, 2,3-dimethylbutylaminocarbonyl, 3,3-dimethylbutylaminocarbonyl, 1-ethylbutylaminocarbonyl, 2-ethylbutylaminocarbonyl, 1,1,2-trimethylpropylaminocarbonyl, 1,2,2-trimethylpropylaminocarbonyl, 1-ethyl-1-methylpropylaminocarbonyl or 1-ethyl-2-methylpropylaminocarbonyl;
di(C₁-C₆-alkyl)aminocarbonyl and also the di(C₁-C₆-alkyl)aminocarbonyl moieties of di(C₁-C₆-alkyl)aminocarbonylamino, di(C₁-C₆-alkyl)aminocarbonyl-C₁-C₄-alkyl, di(C₁-C₆-alkyl)aminocarbonyloxy-C₁-C₄-alkyl and di(C₁-C₆-alkyl)aminocarbonylamino-C₁-C₄-alkyl: di(C₁-C₄-alkyl)aminocarbonyl as mentioned above, and also, for example, N-methyl-N-pentylaminocarbonyl, N-methyl-N-(1-methylbutyl)aminocarbonyl, N-methyl-N-(2-methylbutyl)aminocarbonyl, N-methyl-N-(3-methylbutyl)aminocarbonyl, N-methyl-N-(2,2-dimethylpropyl)aminocarbonyl, N-methyl-N-(1-ethylpropyl)aminocarbonyl, N-methyl-N-hexylaminocarbonyl, N-methyl-N-(1,1-dimethylpropyl)aminocarbonyl, N-methyl-N-(1,2-dimethylpropyl)aminocarbonyl, N-methyl-N-(1-methylpentyl)aminocarbonyl, N-methyl-N-(2-methylpentyl)aminocarbonyl, N-methyl-N-(3-methylpentyl)aminocarbonyl, N-methyl-N-(4-methylpentyl)aminocarbonyl, N-methyl-N-(1,1-dimethylbutyl)aminocarbonyl, N-methyl-N-(1,2-dimethylbutyl)aminocarbonyl, N-methyl-N-(1,3-dimethylbutyl)aminocarbonyl, N-methyl-N-(2,2-dimethylbutyl)aminocarbonyl, N-methyl-N-(2,3-dimethylbutyl)aminocarbonyl, N-methyl-N-(3,3-dimethylbutyl)aminocarbonyl, N-methyl-N-(1-ethylbutyl)aminocarbonyl, N-methyl-N-(2-ethylbutyl)aminocarbonyl, N-methyl-N-(1,1,2-trimethylpropyl)aminocarbonyl, N-methyl-N-(1,2,2-trimethylpropyl)aminocarbonyl, N-methyl-N-(1-ethyl-1-methylpropyl)aminocarbonyl, N-methyl-N-(1-ethyl-2-methylpropyl)aminocarbonyl, N-ethyl-N-pentylaminocarbonyl, N-ethyl-N-(1-methylbutyl)aminocarbonyl, N-ethyl-N-(2-methylbutyl)aminocarbonyl, N-ethyl-N-(3-methylbutyl)aminocarbonyl, N-ethyl-N-(2,2-dimethylpropyl)aminocarbonyl, N-ethyl-N-(1-ethylpropyl)aminocarbonyl, N-ethyl-N-hexylaminocarbonyl, N-ethyl-N-(1,1-dimethylpropyl)aminocarbonyl, N-ethyl-N-(1,2-dimethylpropyl)aminocarbonyl, N-ethyl-N-(1-methylpentyl)aminocarbonyl, N-ethyl-N-(2-methylpentyl)-aminocarbonyl, N-ethyl-N-(3-methylpentyl)aminocarbonyl, N-ethyl-N-(4-methylpentyl)aminocarbonyl, N-ethyl-N-(1,1-dimethylbutyl)aminocarbonyl, N-ethyl-N-(1,2-dimethylbutyl)aminocarbonyl, N-ethyl-N-(1,3-dimethylbutyl)aminocarbonyl, N-ethyl-N-(2,2-dimethylbutyl)aminocarbonyl, N-ethyl-N-(2,3-dimethylbutyl)aminocarbonyl, N-ethyl-N-(3,3-dimethylbutyl)aminocarbonyl, N-ethyl-N-(1-ethylbutyl)-aminocarbonyl, N-ethyl-N-(2-ethylbutyl)aminocarbonyl, N-ethyl-N-(1,1,2-trimethylpropyl)aminocarbonyl, N-ethyl-N-(1,2,2-trimethylpropyl)aminocarbonyl, N-ethyl-N-(1-ethyl-1-methylpropyl)aminocarbonyl, N-ethyl-N-(1-ethyl-2-methylpropyl)-aminocarbonyl, N-propyl-N-pentylaminocarbonyl, N-butyl-N-pentylaminocarbonyl, N,N-dipentylaminocarbonyl, N-propyl-N-hexylaminocarbonyl, N-butyl-N-hexylaminocarbonyl, N-pentyl-N-hexylaminocarbonyl or N,N-dihexylaminocarbonyl;
di(C₁-C₆-alkyl)aminothiocarbonyl: for example N,N-dimethylaminothiocarbonyl, N,N-diethylaminothiocarbonyl, N,N-di-(1-methylethyl)aminothiocarbonyl, N,N-dipropylaminothiocarbonyl, N,N-dibutylaminothiocarbonyl, N,N-di-(1-methylpropyl)aminothiocarbonyl, N,N-di-(2-methylpropyl)aminothiocarbonyl, N,N-di-(1,1-dimethylethyl)aminothiocarbonyl, N-ethyl-N-methylaminothiocarbonyl, N-methyl-N-propylaminothiocarbonyl, N-methyl-N-(1-methylethyl)aminothiocarbonyl, N-butyl-N-methylaminothiocarbonyl, N-methyl-N-(1-methylpropyl)aminothiocarbonyl, N-methyl-N-(2-methylpropyl)aminothiocarbonyl, N-(1,1-dimethylethyl)-N-methylaminothiocarbonyl, N-ethyl-N-propylaminothiocarbonyl, N-ethyl-N-(1-methylethyl)-aminothiocarbonyl, N-butyl-N-ethylaminothiocarbonyl, N-ethyl-N-(1-methylpropyl)-aminothiocarbonyl, N-ethyl-N-(2-methylpropyl)aminothiocarbonyl, N-ethyl-N-(1,1-dimethylethyl)aminothiocarbonyl, N-(1-methylethyl)-N-propylaminothiocarbonyl, N-butyl-N-propylaminothiocarbonyl, N-(1-methylpropyl)-N-propylaminothiocarbonyl, N-(2-methylpropyl)-N-propylamino-thiocarbonyl, N-(1,1-dimethylethyl)-N-propylaminothiocarbonyl, N-butyl-N-(1-methylethyl)aminothiocarbonyl, N-(1-methylethyl)-N-(1-methylpropyl)aminothiocarbonyl, N-(1-methylethyl)-N-(2-methylpropyl)aminothiocarbonyl, N-(1,1-dimethylethyl)-N-(1-methylethyl)aminothiocarbonyl, N-butyl-N-(1-methylpropyl)aminothiocarbonyl, N-butyl-N-(2-methylpropyl)aminothiocarbonyl, N-butyl-N-(1,1-dimethylethyl)aminothiocarbonyl, N-(1-methylpropyl)-N-(2-methylpropyl)aminothiocarbonyl, N-(1,1-dimethylethyl)-N-(1-methylpropyl)aminothiocarbonyl, N-(1,1-dimethylethyl)-N-(2-methylpropyl)-aminothiocarbonyl, N-methyl-N-pentylaminothiocarbonyl, N-methyl-N-(1-methylbutyl)aminothiocarbonyl, N-methyl-N-(2-methylbutyl)aminothiocarbonyl, N-methyl-N-(3-methylbutyl)aminothiocarbonyl, N-methyl-N-(2,2-dimethylpropyl)aminothiocarbonyl, N-methyl-N-(1-ethylpropyl)aminothiocarbonyl, N-methyl-N-hexylaminothiocarbonyl, N-methyl-N-(1,1-dimethylpropyl)aminothiocarbonyl, N-methyl-N-(1,2-dimethylpropyl)aminothiocarbonyl, N-methyl-N-(1-methylpentyl)-aminothiocarbonyl, N-methyl-N-(2-methylpentyl)aminothiocarbonyl, N-methyl-N-(3-methylpentyl)aminothiocarbonyl, N-methyl-N-(4-methylpentyl)aminothiocarbonyl, N-methyl-N-(1,1-dimethylbutyl)aminothiocarbonyl, N-methyl-N-(1,2-dimethylbutyl)aminothiocarbonyl, N-methyl-N-(1,3-dimethylbutyl)aminothiocarbonyl, N-methyl-N-(2,2-dimethylbutyl)aminothiocarbonyl, N-methyl-N-(2,3-dimethylbutyl)aminothiocarbonyl, N-methyl-N-(3,3-dimethylbutyl)aminothiocarbonyl, N-methyl-N-(1-ethylbutyl)aminothiocarbonyl, N-methyl-N-(2-ethylbutyl)-aminothiocarbonyl, N-methyl-N-ethyl-N-(1,1,2-trimethylpropyl)aminothiocarbonyl, N-methyl-N-(1,2,2-trimethylpropyl)aminothiocarbonyl, N-methyl-N-(1-ethyl-1-methylpropyl)aminothiocarbonyl, N-methyl-N-(1-ethyl-2-methylpropyl)aminothiocarbonyl, N-ethyl-N-pentylaminothiocarbonyl, N-ethyl-N-(1-methylbutyl)aminothiocarbonyl, N-ethyl-N-(2-methylbutyl)aminothiocarbonyl, N-ethyl-N-(3-methylbutyl)aminothiocarbonyl, N-ethyl-N-(2,2-dimethylpropyl)aminothiocarbonyl, N-ethyl-N-(1-ethylpropyl)aminothiocarbonyl, N-ethyl-N-hexylaminothiocarbonyl, N-ethyl-N-(1,1-dimethylpropyl)aminothiocarbonyl, N-ethyl-N-(1,2-dimethylpropyl)aminothiocarbonyl, N-ethyl-N-(1-methylpentyl)aminothiocarbonyl, N-ethyl-N-(2-methylpentyl)aminothiocarbonyl, N-ethyl-N-(3-methylpentyl)aminothiocarbonyl, N-ethyl-N-(4-methylpentyl)aminothiocarbonyl, N-ethyl-N-(1,1-dimethylbutyl)aminothiocarbonyl, N-ethyl-N-(1,2-dimethylbutyl)aminothiocarbonyl, N-ethyl-N-(1,3-dimethylbutyl)aminothiocarbonyl, N-ethyl-N-(2,2-dimethylbutyl)aminothiocarbonyl, N-ethyl-N-(2,3-dimethylbutyl)aminothiocarbonyl, N-ethyl-N-(3,3-dimethylbutyl)aminothiocarbonyl, N-ethyl-N-(1-ethylbutyl)aminothiocarbonyl, N-ethyl-N-(2-ethylbutyl)-aminothiocarbonyl, N-ethyl-N-(1,1,2-trimethylpropyl)aminothiocarbonyl, N-ethyl-N-(1,2,2-trimethylpropyl)aminothiocarbonyl, N-ethyl-N-(1-ethyl-1-methylpropyl)-aminothiocarbonyl, N-ethyl-N-(1-ethyl-2-methylpropyl)aminothiocarbonyl, N-propyl-N-pentylaminothiocarbonyl, N-butyl-N-pentylaminothiocarbonyl, N,N-dipentylaminothiocarbonyl, N-propyl-N-hexylaminothiocarbonyl, N-butyl-N-hexylaminothiocarbonyl, N-pentyl-N-hexylaminothiocarbonyl or N,N-dihexylaminothiocarbonyl;
three- to six-membered heterocyclyl: monocyclic saturated or partially unsaturated hydrocarbons having three to six ring members as mentioned above which, in addition to carbon atoms, may contain one to four nitrogen atoms or one to three nitrogen atoms and one oxygen or sulfur atom or one to three oxygen atoms or one to three sulfur atoms and which may be attached via a carbon atom or a nitrogen atom, for example
for example 2-oxiranyl, 2-oxetanyl, 3-oxetanyl, 2-aziridinyl, 3-thiethanyl, 1-azetidinyl, 2-azetidinyl,
for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 1,2,3,4-tetrazolidin-5-yl;
for example 1-pyrrolidinyl, 2-isothiazolidinyl, 2-isothiazolidinyl, 1-pyrazolidinyl, 3-oxazolidinyl, 3-thiazolidinyl, 1-imidazolidinyl, 1,2,4-triazolidin-1-yl, 1,2,4-oxadiazolidin-2-yl, 1,2,4-oxadiazolidin-4-yl, 1,2,4-thiadiazolidin-2-yl, 1,2,4-thiadiazolidin-4-yl, 1,2,3,4-tetrazolidin-1-yl,
for example 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 4,5-dihydropyrrol-2-yl, 4,5-dihydropyrrol-3-yl, 2,5-dihydropyrrol-2-yl, 2,5-dihydropyrrol-3-yl, 4,5-dihydroisoxazol-3-yl, 2,5-dihydroisoxazol-3-yl, 2,3-dihydroisoxazol-3-yl, 4,5-dihydroisoxazol-4-yl, 2,5-dihydroisoxazol-4-yl, 2,3-dihydroisoxazol-4-yl, 4,5-dihydroisoxazol-5-yl, 2,5-dihydroisoxazol-5-yl, 2,3-dihydroisoxazol-5-yl, 4,5-dihydroisothiazol-3-yl, 2,5-dihydroisothiazol-3-yl, 2,3-dihydroisothiazol-3-yl, 4,5-dihydroisothiazol-4-yl, 2,5-dihydroisothiazol-4-yl, 2,3-dihydroisothiazol-4-yl, 4,5-dihydroisothiazol-5-yl, 2,5-dihydroisothiazol-5-yl, 2,3-dihydroisothiazol-5-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-3-yl, 3,4-dihydropyrazol-4-yl, 3,4-dihydropyrazol-5-yl, 4,5-dihydropyrazol-3-yl, 4,5-dihydropyrazol-4-yl, 4,5-dihydropyrazol-5-yl, 2,3-dihydroimidazol-2-yl, 2,3-dihydroimidazol-3-yl, 2,3-dihydroimidazol-4-yl, 2,3-dihydroimidazol-5-yl, 4,5-dihydroimidazol-2-yl, 4,5-dihydroimidazol-4-yl, 4,5-dihydroimidazol-5-yl, 2,5-dihydroimidazol-2-yl, 2,5-dihydroimidazol-4-yl, 2,5-dihydroimidazol-5-yl, 2,3-dihydrooxazol-3-yl, 2,3-dihydrooxazol-4-yl, 2,3-dihydrooxazol-5-yl, 3,4-dihydrooxazol-3-yl, 3,4-dihydrooxazol-4-yl, 3,4-dihydrooxazol-5-yl, 2,3-dihydrothiazol-3-yl, 2,3-dihydrothiazol-4-yl, 2,3-dihydrothiazol-5-yl, 3,4-dihydrothiazol-3-yl, 3,4-dihydrothiazol-4-yl, 3,4-dihydrothiazol-5-yl, 3,4-dihydrothiazol-2-yl, 3,4-dihydrothiazol-3-yl, 3,4-dihydrothiazol-4-yl,
for example 4,5-dihydropyrrol-1-yl, 2,5-dihydropyrrol-1-yl, 4,5-dihydroisoxazol-2-yl, 2,3-dihydroisoxazol-1-yl, 4,5-dihydroisothiazol-1-yl, 2,3-dihydroisothiazol-1-yl, 2,3-dihydropyrazol-1-yl, 4,5-dihydropyrazol-1-yl, 3,4-dihydropyrazol-1-yl, 2,3-dihydroimidazol-1-yl, 4,5-dihydroimidazol-1-yl, 2,5-dihydroimidazol-1-yl, 2,3-dihydrooxazol-2-yl, 3,4-dihydrooxazol-2-yl, 2,3-dihydrothiazol-2-yl, 3,4-dihydrothiazol-2-yl;
for example 2-piperidinyl, 3-piperidinyl, 4-piperidinyl, 1,3-dioxan-2-yl, 1,3-dioxan-4-yl, 1,3-dioxan-5-yl, 1,4-dioxan-2-yl, 1,3-dithian-2-yl, 1,4-dithian-3-yl, 1,3-dithian-4-yl, 1,4,dithian-2-yl 2-tetrahydropyranyl, 3-tetrahydropyranyl, 4-tetrahydropyranyl, 2-tetrahydrothiopyranyl, 3-tetrahydrothiopyranyl, 4-tetrahydrothiopyranyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, tetrahydro-1,3-oxazin-2-yl, tetrahydro-1,3-oxazin-6-yl, 2-morpholinyl, 3-morpholinyl, 1,3,5-trioxan-2-yl;
for example 1-piperidinyl, 1-hexahydropyridazinyl, 1-hexahydropyrimidinyl, 1-piperazinyl, 1,3,5-hexahydrotriazin-1-yl, 1,2,4-hexahydrotriazin-1-yl, tetrahydro-1,3-oxazin-1-yl, 1-morpholinyl;
for example 2H-pyran-2-yl, 2H-pyran-3-yl, 2H-pyran-4-yl, 2H-pyran-5-yl, 2H-pyran-6-yl, 3,6-dihydro-2H-pyran-2-yl, 3,6-dihydro-2H-pyran-3-yl, 3,6-dihydro-2H-pyran-4-yl, 3,6-dihydro-2H-pyran-5-yl, 3,6-dihydro-2H-pyran-6-yl, 3,4-dihydro-2H-pyran-3-yl, 3,4-dihydro-2H-pyran-4-yl, 3,4-dihydro-2H-pyran-6-yl, 2H-thiopyran-2-yl, 2H-thiopyran-3-yl, 2H-thiopyran-4-yl, 2H-thiopyran-5-yl, 2H-thiopyran-6-yl, 5,6-dihydro-4H-1,3-oxazin-2-yl;
aryl and the aryl moiety of aryl(C₁-C₆-alkyl), aryl(C₁-C₄-alkyl): a monocyclic to tricyclic aromatic carbocycle having 6 to 14 ring members, such as, for example, phenyl, naphthyl and anthracenyl;
heteroaryl and also the heteroaryl radicals in heteroaryl-C₁-C₄-alkyl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, heteroarylsulfonyl-C₁-C₄-alkyl:
mono- or bicyclic aromatic heteroaryl having 5 to 10 ring members which, in addition to carbon atoms, contains 1 to 4 nitrogen atoms, or 1 to 3 nitrogen atoms and an oxygen or sulfur atom, or an oxygen or a sulfur atom, for example
monocycles, such as furyl (for example 2-furyl, 3-furyl), thienyl (for example 2-thienyl, 3-thienyl), pyrrolyl (for example pyrrol-2-yl, pyrrol-3-yl), pyrazolyl (for example pyrazol-3-yl, pyrazol-4-yl), isoxazolyl (for example isoxazol-3-yl, isoxazol-4-yl, isoxazol-5-yl), isothiazolyl (for example isothiazol-3-yl, isothiazol-4-yl, isothiazol-5-yl), imidazolyl (for example imidazol-2-yl, imidazol-4-yl), oxazolyl (for example oxazol-2-yl, oxazol-4-yl, oxazol-5-yl), thiazolyl (for example thiazol-2-yl, thiazol-4-yl, thiazol-5-yl), oxadiazolyl (for example 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl), thiadiazolyl (for example 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl), triazolyl (for example 1,2,3-triazol-4-yl, 1,2,4-triazol-3-yl), tetrazol-5-yl, pyridyl (for example pyridin-2-yl, pyridin-3-yl, pyridin-4-yl), pyrazinyl (for example pyridazin-3-yl, pyridazin-4-yl), pyrimidinyl (for example pyrimidin-2-yl, pyrimidin-4-yl, pyrimidin-5-yl), pyrazin-2-yl, triazinyl (for example 1,3,5-triazin-2-yl, 1,2,4-triazin-3-yl, 1,2,4-triazin-5-yl, 1,2,4-triazin-6-yl), tetrazinyl (for example 1,2,4,5-tetrazin-3-yl); and also
bicycles such as the benzo-fused derivatives of the abovementioned monocycles, for example quinolinyl, isoquinolinyl, indolyl, benzothienyl, benzofuranyl, benzoxazolyl, benzothiazolyl, benzisothiazolyl, benzimidazolyl, benzopyrazolyl, benzothiadiazolyl, benzotriazolyl;
5- or 6-membered heteroaryl having one to four nitrogen atoms, or one to three nitrogen atoms and one oxygen or sulfur atom, or having one oxygen or sulfur atom: for example aromatic 5-membered heterocycles which are attached via a carbon atom and which, in addition to carbon atoms, may contain one to four nitrogen atoms, or one to three nitrogen atoms and one oxygen or sulfur atom, or having one oxygen or sulfur atom as ring members, for example, 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
for example aromatic 6-membered heterocycles which are attached via a carbon atom and which, in addition to carbon atoms, may contain one to four, preferably one to three nitrogen atoms as ring members, for example, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In a particular embodiment, the variables of the heteroaroyl-substituted alanines of the formula I have the following meanings which, both on their own and in combination with one another, are particular embodiments of the compounds of the formula I:

Preference is given to the heteroaroyl-substituted alanines of the formula I in which
A is 5- or 6-membered heteroaryl having one to four nitrogen atoms, or one to three nitrogen atoms and one oxygen or sulfur atom, or having one oxygen or sulfur atom, which heteroaryl is substituted by a C₁-C₆-haloalkyl radical,
   preferably substituted in the 2-position by a C₁-C₆-haloalkyl radical, and may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl.

Preference is also given to the heteroaroyl-substituted alanines of the formula I in which
A is 5- or 6-membered heteroaryl selected from the group consisting of pyrrolyl, thienyl, furyl, pyrazolyl, imidazolyl, thiazolyl, oxazolyl, tetrazolyl, pyridyl and pyrimidinyl;
   where the heteroaryl radicals mentioned may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
preferably 5- or 6-membered heteroaryl selected from the group consisting of thienyl, furyl, pyrazolyl, imidazolyl, thiazolyl, oxazolyl and pyridyl;
   where the heteroaryl radicals mentioned may be partially or fully halogenated and/or may carry 1 to 3 radicals from the group consisting of C₁-C₆-alkyl, C₃-C₆-cycloalkyl and C₁-C₆-haloalkyl;
very preferably 5-membered heteroaryl selected from the group consisting of thienyl, furyl, pyrazolyl, imidazolyl, thiazolyl and oxazolyl;
   where the heteroaryl radicals mentioned may be partially halogenated and/or may carry 1 or 2 radicals from the group consisting of C₁-C₆-alkyl and C₁-C₄-haloalkyl;
particularly preferably 5-membered heteroaryl selected from the group consisting of thienyl, furyl, pyrazolyl and imidazolyl;
   where the heteroaryl radicals mentioned may be partially halogenated and/or may carry 1 or 2 radicals from the group consisting of C₁-C₆-alkyl and C₁-C₄-haloalkyl;
especially preferably 5-membered heteroaryl selected from the group consisting of pyrazolyl and imidazolyl;
   where the heteroaryl radicals mentioned may be partially halogenated and/or may carry 1 or 2 radicals from the group consisting of C₁-C₆-alkyl and C₁-C₄-haloalkyl;
most preferably pyrazolyl which may be partially halogenated and/or may carry 1 or 2 radicals from the group consisting of C₁-C₆-alkyl and C₁-C₄-haloalkyl.

Preference is also given to the heteroaroyl-substituted alanines of the formula I in which
A is 6-membered heteroaryl having one to four nitrogen atoms;
   particularly preferably pyridyl or pyrimidyl,
   especially preferably pyrimidyl;
      where the heteroaryl radicals mentioned may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl.

Preference is also given to the heteroaroyl-substituted alanines of the formula I in which
A is 5-membered heteroaryl having one to four nitrogen atoms, or one to three nitrogen atoms and one oxygen or sulfur atom, or having one oxygen atom;
   particularly preferably 5-membered heteroaryl selected from the group consisting of thienyl, furyl, pyrazolyl, imidazolyl, thiazolyl and oxazolyl;
   especially preferably 5-membered heteroaryl selected from the group consisting of thienyl, furyl, pyrazolyl and imidazolyl;
   most preferably pyrazolyl;
      where the heteroaryl radicals mentioned are substituted by a C₁-C₆-haloalkyl radical, preferably in the 2-position by a C₁-C₆-haloalkyl radical, and may carry 1 to 3 radicals from the group consisting of halogen, cyano, C₁-C₆-alkyl, C₃-C₆-cycloalkyl, C₁-C₆-alkoxy, C₁-C₆-haloalkoxy and C₁-C₆-alkoxy-C₁-C₄-alkyl.

Preference is also given to the heteroaroyl-substituted alanines of the formula I in which
A is 5-membered heteroaryl having one to four nitrogen atoms;
   preferably 5-membered heteroaryl having one to three nitrogen atoms;
   very preferably 5-membered heteroaryl having one to two nitrogen atoms;
   particularly preferably 5-membered heteroaryl having two nitrogen atoms;
   most preferably pyrazolyl;
      where the heteroaryl radicals mentioned may be substituted by 1 to 3 radicals from the group consisting of halogen, cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
      preferably by 1 to 3 radicals from the group consisting of halogen, cyano, C₁-C₆-alkyl and C₁-C₆-haloalkyl;
      very preferably by 1 to 2 radicals from the group consisting of C₁-C₆-alkyl and C₁-C₆-haloalkyl.

Preference is also given to the heteroaroyl-substituted alanines of the formula I in which
A is 5- or 6-membered heteroaryl attached via carbon and selected from the group consisting of A1 to A14 where

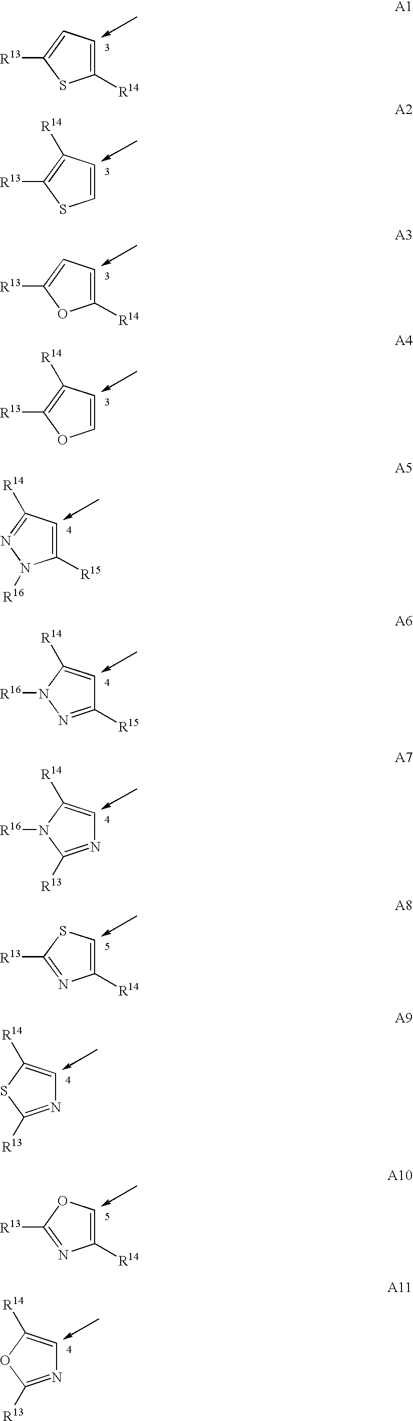

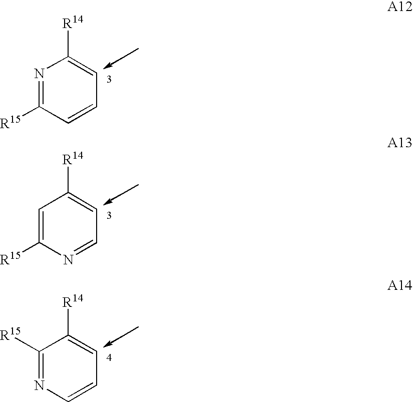

where the arrow indicates the point of attachment and
   R¹³ is hydrogen, halogen, C₁-C₆-alkyl or C₁-C₆-haloalkyl;
      particularly preferably hydrogen, C₁-C₄-alkyl or C₁-C₄-haloalkyl;
      especially preferably hydrogen or C₁-C₄-alkyl;
      most preferably hydrogen;
   R¹⁴ is halogen, C₁-C₆-alkyl, C₁-C₆-haloalkyl or C₁-C₆-haloalkoxy;
      particularly preferably halogen, C₁-C₄-alkyl or C₁-C₆-haloalkyl;
      especially preferably halogen or C₁-C₆-haloalkyl;
      very preferably C₁-C₆-haloalkyl;
      most preferably C₁-C₄-haloalkyl
      with utmost preference CF₃;
   R¹⁵ is hydrogen, halogen, C₁-C₆-alkyl or C₁-C₆-haloalkyl;
      particularly preferably hydrogen, halogen or C₁-C₄-haloalkyl;
      especially preferably hydrogen or halogen;
      most preferably hydrogen; and
   R¹⁶ is hydrogen, C₁-C₆-alkyl, C₃-C₆-cycloalkyl, C₁-C₆-haloalkyl or C₁-C₆-alkoxy-C₁-C₄-alkyl;
      particularly preferably C₁-C₄-alkyl, C₃-C₆-cycloalkyl, C₁-C₄-haloalkyl or C₁-C₄-alkoxy-C₁-C₄-alkyl;
      especially preferably C₁-C₄-alkyl or C₁-C₄-haloalkyl;
      most preferably C₁-C₄-alkyl;
      with utmost preference CH₃;
very preferably A1, A2, A3, A4, A5, A6, A8 or A9;
   where R¹³ to R¹⁶ are as defined above;
particularly preferably A1, A2, A5 or A6;
   where R¹³ to R¹⁶ are as defined above;
especially preferably A5 or A6;
where R¹⁴ to R¹⁶ are as defined above;
most preferably A5;
where R¹⁴ to R¹⁶ are as defined above.

Preference is also given to the heteroaroyl-substituted alanines of the formula I in which
R¹ is hydrogen.

Preference is also given to the heteroaroyl-substituted alanines of the formula I in which
R² is hydrogen or hydroxyl;
   particularly preferably hydrogen.

Preference is also given to the heteroaroyl-substituted alanines of the formula I in which
R³ is C₁-C₆-alkyl or C₁-C₆-haloalkyl;
   particularly preferably C₁-C₆-alkyl;
   especially preferably C₁-C₄-alkyl;
   most preferably CH₃.

Preference is also given to the heteroaroyl-substituted alanines of the formula I in which
R⁴ is hydrogen or C₁-C₄-alkyl;
   preferably hydrogen or CH₃;
   especially preferably hydrogen.

Preference is also given to the heteroaroyl-substituted alanines of the formula I in which
R⁵ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl or 3- to 6-membered heterocyclyl,
   where the cycloalkyl, cycloalkenyl or 3- to 6-membered heterocyclyl radicals mentioned above may be partially or fully halogenated and/or may carry one to three radicals from the group consisting of oxo, C₁-C₆-alkyl, C₁-C₆-haloalkyl, hydroxycarbonyl and C₁-C₆-alkoxycarbonyl;
C₁-C₆-alkoxy-C₁-C₄-alkyl, C₁-C₆-haloalkoxy-C₁-C₄-alkyl, C₁-C₆-alkoxy-C₁-C₄-alkoxy-C₁-C₄-alkyl, C₁-C₆-alkylthio-C₁-C₄-alkyl, C₁-C₆-alkylsulfonylamino-C₁-C₄-alkyl, hydroxycarbonyl, C₁-C₆-alkoxycarbonyl, aminocarbonyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, C₁-C₆-alkylcarbonylamino-C₁-C₄-alkyl, (C₁-C₆-alkyl)aminocarbonylamino-C₁-C₄-alkyl di(C₁-C₆-alkyl)aminocarbonylamino-C₁-C₄-alkyl, di(C₁-C₆-alkyl)aminocarbonyloxy-C₁-C₄-alkyl, formylamino-C₁-C₄-alkyl;
phenyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₁-C₄-hydroxyalkyl, phenyloxy-C₁-C₄-alkyl, phenylthio-C₁-C₄-alkyl, phenylsulfinyl-C₁-C₄-alkyl, phenylsulfonyl-C₁-C₄-alkyl;
heteroaryl, heteroaryl-C₁-C₄-alkyl, heteroaryl-C₁-C₄-hydroxyalkyl, heteroaryloxy-C₁-C₄-alkyl, heteroarylthio-C₁-C₄-alkyl, heteroarylsulfinyl-C₁-C₄-alkyl or heteroarylsulfonyl-C₁-C₄-alkyl,
   where the phenyl and heteroaryl radicals mentioned above may be partially or fully halogenated and/or may carry one to three radicals from the group consisting of cyano, nitro, C₁-C₆-alkyl, C₁-C₆-haloalkyl, hydroxyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkylsulfonylamino and C₁-C₆-haloalkylsulfonylamino;
particularly preferably C₂-C₆-alkenyl, C₁-C₆-haloalkyl, 3- to 6-membered heterocyclyl, C₁-C₆-alkoxy-C₁-C₄-alkyl, C₁-C₆-alkoxy-C₁-C₄-alkoxy-C₁-C₄-alkyl, aminocarbonyl, C₁-C₆-alkylcarbonylamino-C₁-C₄-alkyl, formylamino-C₁-C₄-alkyl, phenyl or heteroaryl,
   where the 3- to 6-membered heterocyclyl and the phenyl and heteroaryl radicals mentioned above may be partially or fully halogenated and/or may carry one to three C₁-C₆-alkyl radicals;
especially preferably C₂-C₆-alkenyl, C₁-C₆-alkoxy-C₁-C₄-alkyl, C₁-C₆-alkoxy-C₁-C₄-alkoxy-C₁-C₄-alkyl, phenyl or heteroaryl.

Preference is also given to the heteroaroyl-substituted alanines of the formula I in which
R⁶ is NR¹⁰R¹¹ or OR⁹
   particularly preferably OR⁹.

Preference is also given to the heteroaroyl-substituted alanines of the formula I in which
R⁶ is NR¹⁰R¹¹ or NO₂;
   particularly preferably NR¹⁰R¹¹.

Preference is also given to the heteroaroyl-substituted alanines of the formula I in which
R⁶ is OR⁹ or NO₂;
   particularly preferably NO₂.

Preference is also given to the heteroaroyl-substituted alanines of the formula I in which
R⁷ is hydrogen or C₁-C₆-alkyl;
   particularly preferably hydrogen or C₁-C₄-alkyl;
   especially preferably hydrogen.

Preference is also given to the heteroaroyl-substituted alanines of the formula I in which
R⁸ is hydrogen or C₁-C₆-alkyl;
   particularly preferably hydrogen or C₁-C₄-alkyl;
   especially preferably hydrogen.

Preference is also given to the heteroaroyl-substituted alanines of the formula I in which
R⁹ and R¹⁰ in each case independently of one another are
   hydrogen, C₁-C₆-alkyl, C₃-C₆-alkenyl, C₃-C₆-alkynyl, formyl, C₁-C₆-alkylcarbonyl, C₂-C₆-alkenylcarbonyl, C₃-C₆-cycloalkylcarbonyl, C₁-C₆-alkoxycarbonyl, aminocarbonyl, C₁-C₆-alkylaminocarbonyl, C₁-C₆-alkylsulfonylaminocarbonyl, di(C₁-C₆-alkyl)aminocarbonyl, N—(C₁-C₆-alkoxy)-N—(C₁-C₆-alkyl)aminocarbonyl, [(C₁-C₆-alkyl)aminocarbonyl(C₁-C₆-alkyl)amino]carbonyl, (C₁-C₆-alkyl)aminothiocarbonyl, di(C₁-C₆-alkyl)aminothiocarbonyl, C₁-C₆-alkoxyimino-C₁-C₆-alkyl,
      where the alkyl, cycloalkyl and alkoxy radicals mentioned may be partially or fully halogenated and/or may carry one to three of the following groups: cyano, hydroxyl, C₃-C₆-cycloalkyl, C₁-C₄-alkoxy, C₁-C₄-alkylthio, di-(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl, or C₁-C₄-alkylcarbonyloxy;
   phenyl, phenyl-C₁-C₆-alkyl, phenylcarbonyl-C₁-C₆-alkyl, phenylsulfonylaminocarbonyl or phenyl-C₁-C₆-alkylcarbonyl,
      where the phenyl ring may be partially or fully halogenated and/or may carry one to three of the following groups: nitro, cyano, C₁-C₄-alkyl, C₁-C₄-haloalkyl, C₁-C₄-alkoxy or C₁-C₄-haloalkoxy; or
   SO₂R¹²;
   particularly preferably hydrogen, C₁-C₆-alkyl, C₃-C₆-alkenyl, C₃-C₆-alkynyl, formyl, C₁-C₆-alkylcarbonyl, C₂-C₆-alkenylcarbonyl, C₁-C₆-alkoxycarbonyl, aminocarbonyl, (C₁-C₆-alkyl)aminocarbonyl, C₁-C₆-alkylsulfonylaminocarbonyl, di(C₁-C₆-alkyl)aminocarbonyl, N—(C₁-C₆-alkoxy)-N—(C₁-C₆-alkyl)aminocarbonyl, [(C₁-C₆-alkyl)aminocarbonyl(C₁-C₆-alkyl)amino]carbonyl, (C₁-C₆-alkyl)aminothiocarbonyl or di(C₁-C₆-alkyl)aminothiocarbonyl,
      where the alkyl or alkoxy radicals mentioned may be partially or fully halogenated and/or may carry one to three of the following groups: C₁-C₄-alkoxy, C₁-C₄-alkoxycarbonyl, C₁-C₄-alkylaminocarbonyl or di(C₁-C₄-alkyl)aminocarbonyl; or
   SO₂R¹²;
   especially preferably hydrogen, C₁-C₆-alkyl, formyl, C₁-C₆-alkylcarbonyl, C₁-C₆-haloalkylcarbonyl, C₁-C₆-alkoxycarbonyl, aminocarbonyl, (C₁-C₆-alkyl)aminocarbonyl, di(C₁-C₆-alkyl)aminocarbonyl, N—(C₁-C₆-alkoxy)-N—(C₁-C₆-alkyl)-aminocarbonyl, [(C₁-C₆-alkyl)aminocarbonyl(C₁-C₆-alkyl)amino]carbonyl or di(C₁-C₆-alkyl)aminothiocarbonyl; or
   SO₂R¹².

Preference is also given to the heteroaroyl-substituted alanines of the formula I in which
R⁹ and R¹⁰ in each case independently of one another are
   hydrogen, C₁-C₆-alkyl, C₃-C₆-alkenyl, C₃-C₆-alkynyl, formyl, C₁-C₆-alkylcarbonyl, C₂-C₆-alkenylcarbonyl, C₃-C₆-cycloalkylcarbonyl, C₁-C₆-alkoxycarbonyl, aminocarbonyl, C₁-C₆-alkylaminocarbonyl, di(C₁-C₆-alkyl)aminocarbonyl, N—(C₁-C₆-alkoxy)-N—(C₁-C₆-alkyl)aminocarbonyl, [(C₁-C₆-alkyl)aminocarbonyl(C₁-C₆-alkyl)amino]carbonyl, di(C₁-C₆-alkyl)aminothiocarbonyl or C₁-C₆-alkoxyimino-C₁-C₆-alkyl,
      where the alkyl, cycloalkyl or alkoxy radicals mentioned may be partially or fully halogenated and/or may carry one to three of the following groups: cyano, hydroxyl, C₃-C₆-cycloalkyl, C₁-C₄-alkoxy, C₁-C₄-alkylthio, di(C₁-C₄-alkyl)amino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl or C₁-C₄-alkylcarbonyloxy; or
   SO₂R¹².

Preference is also given to the heteroaroyl-substituted alanines of the formula I in which
R¹¹ is hydrogen, C₁-C₆-alkyl, hydroxyl or C₁-C₆-alkoxy;
   particularly preferably hydrogen or C₁-C₆-alkyl;
   especially preferably hydrogen or methyl;
   most preferably hydrogen.

Preference is also given to the heteroaroyl-substituted alanines of the formula I in which
R¹² is C₁-C₆-alkyl, C₁-C₆-haloalkyl, di(C₁-C₆-alkyl)amino or phenyl,
   where the phenyl radical may be partially or partially halogenated and/or may be substituted by C₁-C₄-alkyl;
particularly preferably C₁-C₄-alkyl, C₁-C₄-haloalkyl, di(C₁-C₆-alkyl)amino or phenyl;
especially preferably methyl, trifluoromethyl or phenyl.

Preference is also given to the heteroaroyl-substituted alanines of the formula I in which
R¹² is C₁-C₆-alkyl or (C₁-C₆-alkyl)amino;
   particularly preferably C₁-C₄-alkyl or di(C₁-C₄-alkyl)amino.

Particular preference is given to the heteroaroyl-substituted alanines of the formula I in which
A is 5- or 6-membered heteroaryl selected from the group consisting of thienyl, furyl, pyrazolyl, imidazolyl, thiazolyl, oxazolyl and pyridyl; where the
   heteroaryl radicals mentioned may be partially or fully halogenated and/or may carry 1 to 3 radicals from the group consisting of C₁-C₆-alkyl, C₃-C₆-cycloalkyl and C₁-C₆-haloalkyl;
R¹ and R² are hydrogen;
R³ is C₁-C₄-alkyl,
   particularly preferably CH₃;
R⁴ is hydrogen;
R⁵ is C₂-C₆-alkenyl, C₁-C₆-haloalkyl, 3- to 6-membered heterocyclyl, C₁-C₆-alkoxy-C₁-C₄-alkyl, C₁-C₆-alkoxy-C₁-C₄-alkoxy-C₁-C₄-alkyl, aminocarbonyl, C₁-C₆-alkylcarbonylamino-C₁-C₄-alkyl, formylamino-C₁-C₄-alkyl, phenyl or heteroaryl, where the 3- to 6-membered heterocyclyl and the phenyl and heteroaryl radicals mentioned above may be partially or fully halogenated and/or may carry one to three C₁-C₆-alkyl radicals;
R⁷ and R⁸ are hydrogen;
R⁹ and R¹⁰ are hydrogen, C₁-C₆-alkyl, formyl, C₁-C₆-alkylcarbonyl, C₁-C₆-haloalkylcarbonyl, C₁-C₆-alkoxycarbonyl, aminocarbonyl, (C₁-C₆-alkyl)aminocarbonyl, di(C₁-C₆-alkyl)aminocarbonyl, N—(C₁-C₆-alkoxy)-N—(C₁-C₆-alkyl)aminocarbonyl, [(C₁-C₆-alkyl)aminocarbonyl(C₁-C₆-alkyl)amino]carbonyl or di(C₁-C₆-alkyl)aminothiocarbonyl or SO₂R¹²; and
R¹¹ is hydrogen.

Extraordinary preference is given to the compounds of the formula I.a (corresponds to formula I where A=A-1 where R¹³═H, R¹⁴═CF₃, R¹, R², R⁴, R⁷ and R⁸═H; R³═CH₃), especially the compounds of the formulae I.a.1 to I.a.384 of Table 1, where the definitions of the variables A and R¹ to R¹⁶ are of particular importance for the compounds according to the invention not only in combination with one another but in each case also on their own.

TABLE 1

| | |
|---|---|
| | I.a |
| 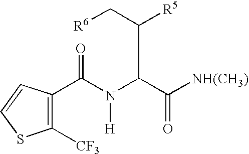 | |

| No. | R⁵ | R⁶ |
|---|---|---|
| I.a.1 | H | OH |
| I.a.2 | CH₃ | OH |
| I.a.3 | CH═CH₂ | OH |
| I.a.4 | CH═CH(CH₃) | OH |
| I.a.5 | CH═CH(CH₃)₂ | OH |
| I.a.6 | C(CH₃)CH₂ | OH |
| I.a.7 | C(CH₃)CH(CH₃) | OH |
| I.a.8 | C≡CH | OH |
| I.a.9 | C≡CCH₃ | OH |
| I.a.10 | CF₃ | OH |
| I.a.11 | CHF₂ | OH |
| I.a.12 | CF₂CF₃ | OH |
| I.a.13 | CH₂OH | OH |
| I.a.14 | CH₂OCH₃ | OH |
| I.a.15 | CH₂OC(O)CH₃ | OH |
| I.a.16 | CH(OH)CH₂OH | OH |
| I.a.17 | CH₂NHC(O)H | OH |
| I.a.18 | CH₂NHC(O)CH₃ | OH |
| I.a.19 | cyclopropyl | OH |
| I.a.20 | cyclopentyl | OH |
| I.a.21 | cyclohexyl | OH |
| I.a.22 | cyclopenten-2-yl | OH |
| I.a.23 | cyclohexen-2-yl | OH |
| I.a.24 | tetrahydrofur-2-yl | OH |
| I.a.25 | tetrahydrofur-3-yl | OH |
| I.a.26 | tetrahydropyran-2-yl | OH |
| I.a.27 | tetrahydropyran-3-yl | OH |
| I.a.28 | tetrahydropyran-4-yl | OH |
| I.a.29 | 3,6-dihydro-2H-pyran-4-yl | OH |
| I.a.30 | 3,6-dihydro-2H-pyran-5-yl | OH |
| I.a.31 | phenyl | OH |
| I.a.32 | pyrid-2-yl | OH |
| I.a.33 | H | OC(O)H |
| I.a.34 | CH₃ | OC(O)H |
| I.a.35 | CH═CH₂ | OC(O)H |
| I.a.36 | CH═CH(CH₃) | OC(O)H |
| I.a.37 | CH═C(CH₃)₂ | OC(O)H |
| I.a.38 | C(CH₃)═CH₂ | OC(O)H |
| I.a.39 | C(CH₃)═CH(CH₃) | OC(O)H |
| I.a.40 | C≡CH | OC(O)H |
| I.a.41 | C≡CCH₃ | OC(O)H |
| I.a.42 | CF₃ | OO(O)H |
| I.a.43 | CHF₂ | OC(O)H |
| I.a.44 | CF₂CF₃ | OC(O)H |
| I.a.45 | CH₂OH | OO(O)H |
| I.a.46 | CH₂OCH₃ | OO(O)H |
| I.a.47 | CH₂OC(O)CH₃ | OC(O)H |
| I.a.48 | CH(OH)CH₂OH | OC(O)H |
| I.a.49 | CH₂NHC(O)H | OC(O)H |
| I.a.50 | CH₂NHC(O)CH₃ | OC(O)H |
| I.a.51 | cyclopropyl | OC(O)H |
| I.a.52 | cyclopentyl | OC(O)H |
| I.a.53 | cyclohexyl | OC(O)H |
| I.a.54 | cyclopenten-2-yl | OC(O)H |
| I.a.55 | cyclohexen-2-yl | OC(O)H |
| I.a.56 | tetrahydrofur-2-yl | OC(O)H |
| I.a.57 | tetrahydrofur-3-yl | OC(O)H |
| I.a.58 | tetrahydropyran-2-yl | OC(O)H |
| I.a.59 | tetrahydropyran-3-yl | OC(O)H |
| l.a.60 | tetrahydropyran-4-yl | OC(O)H |
| I.a.61 | 3,6-dihydro-2H-pyran-4-yl | OC(O)H |
| I.a.62 | 3,6-dihydro-2H-pyran-5-yl | OC(O)H |
| I.a.63 | phenyl | OC(O)H |
| I.a.64 | pyrid-2-yl | OC(O)H |
| I.a.65 | H | OC(O)CH₃ |
| I.a.66 | OH₃ | OC(O)CH₃ |
| I.a.67 | CH═CH₂ | OC(O)CH₃ |
| I.a.68 | CH═CH(CH₃) | OC(O)CH₃ |
| I.a.69 | CH═C(CH₃)₂ | OC(O)CH₃ |
| I.a.70 | C(CH₃)═CH₂ | OC(O)CH₃ |
| l.a.71 | C(CH₃)═CH(CH₃) | OC(O)CH₃ |
| I.a.72 | C≡CH | OC(O)CH₃ |
| I.a.73 | C≡CCH₃ | OC(O)CH₃ |
| I.a.74 | CF₃ | OC(O)CH₃ |
| I.a.75 | CHF₂ | OC(O)CH₃ |
| l.a.76 | CF₂CF₃ | OC(O)CH₃ |
| I.a.77 | CH₂OH | OC(O)CH₃ |
| I.a.78 | CH₂OCH₃ | OC(O)CH₃ |
| I.a.79 | CH₂OC(O)CH₃ | OC(O)CH₃ |
| I.a.80 | CH(OH)CH₂OH | OC(O)CH₃ |
| l.a.81 | CH₂NHC(O)H | OC(O)CH₃ |
| I.a.82 | CH₂NHC(O)CH₃ | OC(O)CH₃ |
| I.a.83 | cyclopropyl | OC(O)CH₃ |
| l.a.84 | cyclopentyl | OC(O)CH₃ |
| I.a.85 | cyclohexyl | OO(O)CH₃ |
| I.a.86 | cyclopenten-2-yl | OC(O)CH₃ |
| I.a.87 | cyclohexen-2-yl | OC(O)CH₃ |
| I.a.88 | tetrahydrofur-2-yl | OC(O)CH₃ |
| I.a.89 | tetrahydrofur-3-yl | OC(O)CH₃ |
| I.a.90 | tetrahydropyran-2-yl | OC(O)CH₃ |
| I.a.91 | tetrahydropyran-3-yl | OC(O)CH₃ |
| I.a.92 | tetrahydropyran-4-yl | OC(O)CH₃ |
| I.a.93 | 3,6-dihydro-2H-pyran-4-yl | OC(O)CH₃ |
| I.a.94 | 3,6-dihydro-2H-pyran-5-yl | OC(O)CH₃ |
| I.a.95 | phenyl | OC(O)CH₃ |
| I.a.96 | pyrid-2-yl | OC(O)CH₃ |
| I.a.97 | H | OC(O)NH(CH₃) |
| I.a.98 | CH₃ | OC(O)NH(CH₃) |
| I.a.99 | CH═CH₂ | OC(O)NH(CH₃) |
| I.a.100 | CH═CH(CH₃) | OC(O)NH(CH₃) |
| I.a.101 | CH═C(CH₃)₂ | OC(O)NH(CH₃) |
| I.a.102 | C(CH₃)═CH₂ | OC(O)NH(CH₃) |
| I.a.103 | C(CH₃)═CH(CH₃) | OC(O)NH(CH₃) |
| I.a.104 | C≡CH | OC(O)NH(CH₃) |
| l.a.105 | C≡CH₃ | OC(O)NH(CH₃) |
| I.a.106 | CF₃ | OC(O)NH(CH₃) |
| I.a.107 | CHF₂ | OC(O)NH(CH₃) |
| I.a.108 | CF₂CF₃ | OC(O)NH(CH₃) |
| I.a.109 | CH₂OH | OC(O)NH(CH₃) |
| I.a.110 | CH₂OCH₃ | OC(O)NH(CH₃) |
| I.a.111 | CH₂OC(O)CH₃ | OC(O)NH(CH₃) |
| I.a.112 | CH(OH)CH₂OH | OC(O)NH(CH₃) |
| I.a.113 | CH₂NHC(O)H | OC(O)NH(CH₃) |
| I.a.114 | CH₂NHC(O)CH₃ | OC(O)NH(CH₃) |
| I.a.115 | cyclopropyl | OC(O)NH(CH₃) |
| I.a.116 | cyclopentyl | OC(O)NH(CH₃) |
| I.a.117 | cyclohexyl | OC(O)NH(CH₃) |
| I.a.118 | cyclopenten-2-yl | OC(O)NH(CH₃) |
| I.a.119 | cyclohexen-2-yl | OC(O)NH(CH₃) |
| I.a.120 | tetrahydrofur-2-yl | OC(O)NH(CH₃) |
| I.a.121 | tetrahydrofur-3-yl | OC(O)NH(CH₃) |
| I.a.122 | tetrahydropyran-2-yl | OC(O)NH(CH₃) |
| I.a.123 | tetrahydropyran-3-yl | OC(O)NH(CH₃) |
| I.a.124 | tetrahydropyran-4-yl | OC(O)NH(CH₃) |
| I.a.125 | 3,6-dihydro-2H-pyran-4-yl | OC(O)NH(CH₃) |
| I.a.126 | 3,6-dihydro-2H-pyran-5-yl | OC(O)NH(CH₃) |
| I.a.127 | phenyl | OC(O)NH(CH₃) |
| I.a.128 | pyrid-2-yl | OC(O)NH(CH₃) |
| I.a.129 | H | OC(O)N(CH₃)₂ |
| I.a.130 | CH₃ | OC(O)N(CH₃)₂ |
| I.a.131 | CH═CH₂ | OC(O)N(CH₃)₂ |
| I.a.132 | CH═CH(CH₃) | OC(O)N(CH₃)₂ |
| I.a.133 | CH═C(CH₃)₂ | OC(O)N(CH₃)₂ |
| I.a.134 | C(CH₃)═CH₂ | OC(O)N(CH₃)₂ |
| I.a.135 | C(CH₃)═CH(CH₃) | OC(O)N(CH₃)₂ |
| I.a.136 | C≡CH | OC(O)N(CH₃)₂ |
| I.a.137 | C≡CCH₃ | OC(O)N(CH₃)₂ |
| I.a.138 | CF₃ | OC(O)N(CH₃)₂ |
| I.a.139 | CHF₂ | OC(O)N(CH₃)₂ |
| I.a.140 | CF₂CF₃ | OC(O)N(CH₃)₂ |
| I.a.141 | CH₂OH | OC(O)N(CH₃)₂ |
| I.a.142 | CH₂OCH₃ | OC(O)N(CH₃)₂ |
| I.a.143 | CH₂OC(O)CH₃ | OC(O)N(CH₃)₂ |
| I.a.144 | CH(OH)CH₂OH | OC(O)N(CH₃)₂ |
| I.a.145 | CH₂NHC(O)H | OC(O)N(CH₃)₂ |
| I.a.146 | CH₂NHC(O)CH₃ | OC(O)N(CH₃)₂ |
| I.a.147 | cyclopropyl | OC(O)N(CH₃)₂ |
| I.a.148 | cyclopentyl | OC(O)N(CH₃)₂ |
| I.a.149 | cyclohexyl | OC(O)N(CH₃)₂ |
| I.a.150 | cyclopenten-2-yl | OC(O)N(CH₃)₂ |
| I.a.151 | cyclohexen-2-yl | OC(O)N(CH₃)₂ |
| I.a.152 | tetrahydrofur-2-yl | OC(O)N(CH₃)₂ |
| I.a.153 | tetrahydrofur-3-yl | OC(O)N(CH₃)₂ |
| I.a.154 | tetrahydropyran-2-yl | OC(O)N(CH₃)₂ |
| I.a.155 | tetrahydropyran-3-yl | OC(O)N(CH₃)₂ |
| I.a.156 | tetrahydropyran-4-yl | OC(O)N(CH₃)₂ |
| I.a.157 | 3,6-dihydro-2H-pyran-4-yl | OC(O)N(CH₃)₂ |
| I.a.158 | 3,6-dihydro-2H-pyran-5-yl | OC(O)N(CH₃)₂ |
| I.a.159 | phenyl | OC(O)N(CH₃)₂ |
| I.a.160 | pyrid-2-yl | OC(O)N(CH₃)₂ |
| I.a.161 | H | OC(O)OCH₃ |
| I.a.162 | CH₃ | OC(O)OCH₃ |
| I.a.163 | CH═CH₂ | OC(O)OCH₃ |
| I.a.164 | CH═CH(CH₃) | OC(O)OCH₃ |
| I.a.165 | CH═C(CH₃)₂ | OC(O)OCH₃ |
| I.a.166 | C(CH₃)═CH₂ | OC(O)OCH₃ |
| I.a.167 | C(CH₃)═CH(CH₃) | OC(O)OCH₃ |
| I.a.168 | C≡CH | OC(O)OCH₃ |
| I.a.169 | C≡CCH₃ | OC(O)OCH₃ |
| I.a.170 | CF₃ | OC(O)OCH₃ |
| I.a.171 | CHF₂ | OC(O)OCH₃ |
| I.a.172 | CF₂CF₃ | OC(O)OCH₃ |
| I.a.173 | CH₂OH | OC(O)OCH₃ |
| I.a.174 | CH₂OCH₃ | OC(O)OCH₃ |
| I.a.175 | CH₂OC(O)CH₃ | OC(O)OCH₃ |
| I.a.176 | CH(OH)CH₂OH | OC(O)OCH₃ |
| I.a.177 | CH₂NHC(O)H | OC(O)OCH₃ |
| I.a.178 | CH₂NHC(O)CH₃ | OC(O)OCH₃ |
| I.a.179 | cyclopropyl | OC(O)OCH₃ |
| I.a.180 | cyclopentyl | OC(O)OCH₃ |
| I.a.181 | cyclohexyl | OC(O)OCH₃ |
| I.a.182 | cyclopenten-2-yl | OC(O)OCH₃ |
| I.a.183 | cyclohexen-2-yl | OC(O)OCH₃ |
| I.a.184 | tetrahydrofur-2-yl | OC(O)OCH₃ |
| I.a.185 | tetrahydrofur-3-yl | OC(O)OCH₃ |
| I.a.186 | tetrahydropyran-2-yl | OC(O)OCH₃ |
| I.a.187 | tetrahydropyran-3-yl | OC(O)OCH₃ |
| I.a.188 | tetrahydropyran-4-yl | OC(O)OCH₃ |
| I.a.189 | 3,6-dihydro-2H-pyran-4-yl | OC(O)OCH₃ |
| I.a.190 | 3,6-dihydro-2H-pyran-5-yl | OC(O)OCH₃ |
| I.a.191 | phenyl | OC(O)OCH₃ |
| I.a.192 | pyrid-2-yl | OC(O)OCH₃ |
| I.a.193 | H | NH₂ |
| I.a.194 | CH₃ | NH₂ |
| I.a.195 | CH═CH₂ | NH₂ |
| I.a.196 | CH═CH(CH₃) | NH₂ |
| I.a.197 | CH═C(CH₃)₂ | NH₂ |
| I.a.198 | C(CH₃)═CH₂ | NH₂ |
| I.a.199 | C(CH₃)═CH(CH₃) | NH₂ |
| I.a.200 | C≡CH | NH₂ |
| I.a.201 | C≡CCH₃ | NH₂ |
| I.a.202 | CF₃ | NH₂ |
| I.a.203 | CHF₂ | NH₂ |
| I.a.204 | CF₂CF₃ | NH₂ |
| I.a.205 | CH₂OH | NH₂ |
| I.a.206 | CH₂OCH₃ | NH₂ |
| I.a.207 | CH₂OC(O)CH₃ | NH₂ |
| I.a.208 | CH(OH)CH₂OH | NH₂ |
| I.a.209 | CH₂NHC(O)H | NH₂ |
| I.a.210 | CH₂NHC(O)CH₃ | NH₂ |
| I.a.211 | cyclopropyl | NH₂ |
| I.a.212 | cyclopentyl | NH₂ |
| I.a.213 | cyclohexyl | NH₂ |
| I.a.214 | cyclopenten-2-yl | NH₂ |
| I.a.215 | cyclohexen-2-yl | NH₂ |
| I.a.216 | tetrahydrofur-2-yl | NH₂ |
| I.a.217 | tetrahydrofur-3-yl | NH₂ |
| I.a.218 | tetrahydropyran-2-yl | NH₂ |
| I.a.219 | tetrahydropyran-3-yl | NH₂ |
| I.a.220 | tetrahydropyran-4-yl | NH₂ |
| I.a.221 | 3,6-dihydro-2H-pyran-4-yl | NH₂ |
| I.a.222 | 3,6-dihydro-2H-pyran-5-yl | NH₂ |
| I.a.223 | phenyl | NH₂ |
| I.a.224 | pyrid-2-yl | NH₂ |
| I.a.225 | H | NHC(O)H |
| I.a.226 | CH₃ | NHC(O)H |
| I.a.227 | CH═CH₂ | NHC(O)H |
| I.a.228 | CH═CH(CH₃) | NHC(O)H |
| I.a.229 | CH═C(CH₃)₂ | NHC(O)H |
| I.a.230 | C(CH₃)═CH₂ | NHC(O)H |
| I.a.231 | C(CH₃)═CH(CH₃) | NHC(O)H |
| I.a.232 | C≡CH | NHC(O)H |
| I.a.233 | C≡CCH₃ | NHC(O)H |
| I.a.234 | CF₃ | NHO(O)H |
| I.a.235 | CHF₂ | NHC(O)H |
| I.a.236 | CF₂CF₃ | NHC(O)H |
| I.a.237 | CH₂OH | NHC(O)H |
| I.a.238 | CH₂OCH₃ | NHC(O)H |
| I.a.239 | CH₂OC(O)CH₃ | NHC(O)H |
| I.a.240 | CH(OH)CH₂OH | NHC(O)H |
| I.a.241 | CH₂NHC(O)H | NHC(O)H |
| I.a.242 | CH₂NHC(O)CH₃ | NHC(O)H |
| I.a.243 | cyclopropyl | NHC(O)H |
| I.a.244 | cyclopentyl | NHC(O)H |
| I.a.245 | cyclohexyl | NHC(O)H |
| I.a.246 | cyclopenten-2-yl | NHC(O)H |
| I.a.247 | cyclohexen-2-yl | NHC(O)H |
| I.a.248 | tetrahydrofur-2-yl | NHC(O)H |
| I.a.249 | tetrahydrofur-3-yl | NHC(O)H |
| I.a.250 | tetrahydropyran-2-yl | NHC(O)H |
| I.a.251 | tetrahydropyran-3-yl | NHC(O)H |
| I.a.252 | tetrahydropyran-4-yl | NHC(O)H |
| I.a.253 | 3,6-dihydro-2H-pyran-4-yl | NHC(O)H |
| I.a.254 | 3,6-dihydro-2H-pyran-5-yl | NHC(O)H |
| I.a.255 | phenyl | NHC(O)H |
| I.a.256 | pyrid-2-yl | NHC(O)H |
| I.a.257 | H | NHC(O)CH₃ |
| I.a.258 | CH₃ | NHC(O)CH₃ |
| I.a.259 | CH═CH₂ | NHC(O)CH₃ |
| I.a.260 | CH═CH(CH₃) | NHC(O)CH₃ |
| I.a.261 | CH═C(CH₃)₂ | NHC(O)CH₃ |
| I.a.262 | C(CH₃)═CH₂ | NHC(O)CH₃ |
| I.a.263 | C(CH₃)═CH(CH₃) | NHC(O)CH₃ |
| I.a.264 | C≡OH | NHC(O)CH₃ |
| I.a.265 | C≡CCH₃ | NHC(O)CH₃ |
| I.a.266 | CF₃ | NHC(O)CH₃ |
| I.a.267 | CHF₂ | NHC(O)CH₃ |
| I.a.268 | CF₂CF₃ | NHC(O)CH₃ |
| I.a.269 | CH₂OH | NHC(O)CH₃ |
| I.a.270 | CH₂OCH₃ | NHC(O)CH₃ |
| I.a.271 | CH₂OC(O)CH₃ | NHC(O)CH₃ |
| I.a.272 | CH(OH)CH₂OH | NHC(O)CH₃ |
| I.a.273 | CH₂NHC(O)H | NHC(O)CH₃ |
| I.a.274 | CH₂NHC(O)CH₃ | NHC(O)CH₃ |
| I.a.275 | cyclopropyl | NHC(O)CH₃ |
| I.a.276 | cyclopentyl | NHC(O)CH₃ |
| I.a.277 | cyclohexyl | NHC(O)CH₃ |
| I.a.278 | cyclopenten-2-yl | NHC(O)CH₃ |
| I.a.279 | cyclohexen-2-yl | NHC(O)CH₃ |
| I.a.280 | tetrahydrofur-2-yl | NHC(O)CH₃ |
| I.a.281 | tetrahydrofur-3-yl | NHC(O)CH₃ |
| I.a.282 | tetrahydropyran-2-yl | NHC(O)CH₃ |
| I.a.283 | tetrahydropyran-3-yl | NHC(O)CH₃ |
| I.a.284 | tetrahydropyran-4-yl | NHC(O)CH₃ |
| I.a.285 | 3,6-dihydro-2H-pyran-4-yl | NHC(O)CH₃ |
| I.a.286 | 3,6-dihydro-2H-pyran-5-yl | NHC(O)CH₃ |
| I.a.287 | phenyl | NHC(O)CH₃ |
| I.a.288 | pyrid-2-yl | NHC(O)CH₃ |
| I.a.289 | H | NHC(O)NH(CH₃) |
| I.a.290 | CH₃ | NHC(O)NH(CH₃) |
| I.a.291 | CH═CH₂ | NHC(O)NH(CH₃) |
| I.a.292 | CH═CH(CH₃) | NHC(O)NH(CH₃) |
| I.a.293 | CH═C(CH₃)₂ | NHC(O)NH(CH₃) |
| I.a.294 | C(CH₃)═CH₂ | NHC(O)NH(CH₃) |
| I.a.295 | C(CH₃)═CH(CH₃) | NHC(O)NH(CH₃) |
| I.a.296 | C≡CH | NHC(O)NH(CH₃) |
| I.a.297 | C≡CCH₃ | NHC(O)NH(CH₃) |
| I.a.298 | CF₃ | NHC(O)NH(CH₃) |
| I.a.299 | CHF₂ | NHC(O)NH(CH₃) |
| I.a.300 | CF₂CF₃ | NHC(O)NH(CH₃) |
| I.a.301 | CH₂OH | NHC(O)NH(CH₃) |
| I.a.302 | CH₂OCH₃ | NHC(O)NH(CH₃) |
| I.a.303 | CH₂OC(O)CH₃ | NHC(O)NH(CH₃) |
| I.a.304 | CH(OH)CH₂OH | NHC(O)NH(CH₃) |
| I.a.305 | CH₂NHC(O)H | NHC(O)NH(CH₃) |
| I.a.306 | CH₂NHC(O)CH₃ | NHC(O)NH(CH₃) |
| I.a.307 | cyclopropyl | NHC(O)NH(CH₃) |
| I.a.308 | cyclopentyl | NHC(O)NH(CH₃) |
| I.a.309 | cyclohexyl | NHC(O)NH(CH₃) |
| I.a.310 | cyclopenten-2-yl | NHC(O)NH(CH₃) |
| I.a.311 | cyclohexen-2-yl | NHC(O)NH(CH₃) |
| I.a.312 | tetrahydrofur-2-yl | NHC(O)NH(CH₃) |
| I.a.313 | tetrahydrofur-3-yl | NHC(O)NH(CH₃) |
| I.a.314 | tetrahydropyran-2-yl | NHC(O)NH(CH₃) |
| I.a.315 | tetrahydropyran-3-yl | NHC(O)NH(CH₃) |
| I.a.316 | tetrahydropyran-4-yl | NHC(O)NH(CH₃) |
| I.a.317 | 3,6-dihydro-2H-pyran-4-yl | NHC(O)NH(CH₃) |
| I.a.318 | 3,6-dihydro-2H-pyran-5-yl | NHC(O)NH(CH₃) |
| I.a.319 | phenyl | NHC(O)NH(CH₃) |
| I.a.320 | pyrid-2-yl | NHC(O)NH(CH₃) |
| I.a.321 | H | NHC(O)N(CH₃)₂ |
| I.a.322 | CH₃ | NHC(O)N(CH₃)₂ |
| I.a.323 | CH═CH₂ | NHC(O)N(CH₃)₂ |
| I.a.324 | CH═CH(CH₃) | NHC(O)N(CH₃)₂ |
| I.a.325 | CH═C(CH₃)₂ | NHC(O)N(CH₃)₂ |
| I.a.326 | C(CH₃)═CH₂ | NHC(O)N(CH₃)₂ |
| I.a.327 | C(CH₃)═CH(CH₃) | NHC(O)N(CH₃)₂ |
| I.a.328 | C≡CH | NHC(O)N(CH₃)₂ |
| I.a.329 | C≡CCH₃ | NHC(O)N(CH₃)₂ |
| I.a.330 | CF₃ | NHC(O)N(CH₃)₂ |
| I.a.331 | CHF₂ | NHC(O)N(CH₃)₂ |
| I.a.332 | CF₂CF₃ | NHC(O)N(CH₃)₂ |
| I.a.333 | CH₂OH | NHC(O)N(CH₃)₂ |
| I.a.334 | CH₂OCH₃ | NHC(O)N(CH₃)₂ |
| I.a.335 | CH₂OC(O)CH₃ | NHC(O)N(CH₃)₂ |
| I.a.336 | CH(OH)CH₂OH | NHC(O)N(CH₃)₂ |
| I.a.337 | CH₂NHC(O)H | NHC(O)N(CH₃)₂ |
| I.a.338 | CH₂NHC(O)CH₃ | NHC(O)N(CH₃)₂ |
| I.a.339 | cyclopropyl | NHC(O)N(CH₃)₂ |
| I.a.340 | cyclopentyl | NHC(O)N(CH₃)₂ |
| I.a.341 | cyclohexyl | NHC(O)N(CH₃)₂ |
| I.a.342 | cyclopenten-2-yl | NHC(O)N(CH₃)₂ |
| I.a.343 | cyclohexen-2-yl | NHC(O)N(CH₃)₂ |
| I.a.344 | tetrahydrofur-2-yl | NHC(O)N(CH₃)₂ |
| I.a.345 | tetrahydrofur-3-yl | NHC(O)N(CH₃)₂ |
| I.a.346 | tetrahydropyran-2-yl | NHC(O)N(CH₃)₂ |
| I.a.347 | tetrahydropyran-3-yl | NHC(O)N(CH₃)₂ |
| I.a.348 | tetrahydropyran-4-yl | NHC(O)N(CH₃)₂ |
| I.a.349 | 3,6-dihydro-2H-pyran-4-yl | NHC(O)N(CH₃)₂ |
| I.a.350 | 3,6-dihydro-2H-pyran-5-yl | NHC(O)N(CH₃)₂ |
| I.a.351 | phenyl | NHC(O)N(CH₃)₂ |
| I.a.352 | pyrid-2-yl | NHC(O)N(CH₃)₂ |
| I.a.353 | H | NHC(O)OCH₃ |
| I.a.354 | CH₃ | NHC(O)OCH₃ |
| I.a.355 | CH═CH₂ | NHC(O)OCH₃ |
| I.a.356 | CH═CH(CH₃) | NHC(O)OCH₃ |
| I.a.357 | CH═C(CH₃)₂ | NHC(O)OCH₃ |
| I.a.358 | C(CH₃)═CH₂ | NHC(O)OCH₃ |
| I.a.359 | C(CH₃)═CH(CH₃) | NHC(O)OCH₃ |
| I.a.360 | C≡CH | NHC(O)OCH₃ |
| I.a.361 | C≡CCH₃ | NHC(O)OCH₃ |
| I.a.362 | CF₃ | NHC(O)OCH₃ |
| I.a.363 | CHF₂ | NHC(O)OCH₃ |
| I.a.364 | CF₂CF₃ | NHC(O)OCH₃ |
| I.a.365 | CH₂OH | NHC(O)OCH₃ |
| I.a.366 | CH₂OCH₃ | NHC(O)OCH₃ |
| I.a.367 | CH₂OC(O)CH₃ | NHC(O)OCH₃ |
| I.a.368 | CH(OH)CH₂OH | NHC(O)OCH₃ |
| I.a.369 | CH₂NHC(O)H | NHC(O)OCH₃ |
| I.a.370 | CH₂NHO(O)CH₃ | NHO(O)OCH₃ |
| I.a.371 | cyclopropyl | NHC(O)OCH₃ |
| I.a.372 | cyclopentyl | NHC(O)OCH₃ |
| I.a.373 | cyclohexyl | NHC(O)OCH₃ |
| I.a.374 | cyclopenten-2-yl | NHC(O)OCH₃ |
| I.a.375 | cyclohexen-2-yl | NHC(O)OCH₃ |
| I.a.376 | tetrahydrofur-2-yl | NHC(O)OCH₃ |
| I.a.377 | tetrahydrofur-3-yl | NHC(O)OCH₃ |
| I.a.378 | tetrahydropyran-2-yl | NHC(O)OCH₃ |
| I.a.379 | tetrahydropyran-3-yl | NHC(O)OCH₃ |
| I.a.380 | tetrahydropyran-4-yl | NHC(O)OCH₃ |
| I.a.381 | 3,6-dihydro-2H-pyran-4-yl | NHC(O)OCH₃ |
| I.a.382 | 3,6-dihydro-2H-pyran-5-yl | NHC(O)OCH₃ |
| I.a.383 | phenyl | NHC(O)OCH₃ |
| I.a.384 | pyrid-2-yl | NHC(O)OCH₃ |

Most preference is also given to the compounds of the formula I.b, especially the compounds of the formulae I.b.1 to I.b.384 which differ from the corresponding compounds of the formulae I.a.1 to I.a.384 in that A is A1 where R¹³═CH₃ and R¹⁴═CF₃:

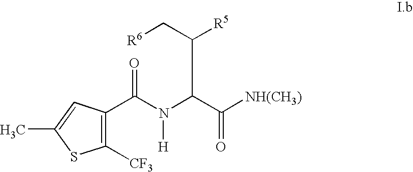

Most preference is also given to the compounds of the formula I.c, especially the compounds of the formulae I.c.1 to I.c.384 which differ from the corresponding compounds of the formulae I.a.1 to I.a.384 in that A is A2 where R¹³═H and R¹⁴═CF₃:

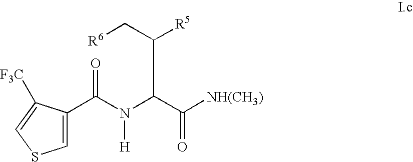

Most preference is also given to the compounds of the formula I.d, especially the compounds of the formulae I.d.1 to I.d.384 which differ from the corresponding compounds of the formulae I.a.1 to I.a.384 in that A is A3 where R¹³═H and R¹⁴═CF₃:

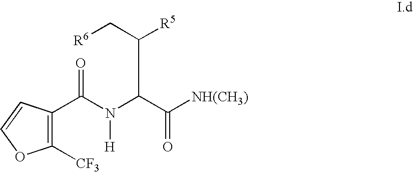

Most preference is also given to the compounds of the formula I.e, especially the compounds of the formulae I.e.1 to I.e.384 which differ from the corresponding compounds of the formulae I.a.1 to I.a.384 in that A is A3 where R¹³═CH₃ and R¹⁴═CF₃:

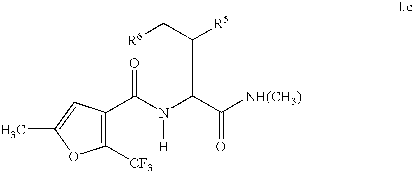

Most preference is also given to the compounds of the formula I.f, especially the compounds of the formulae I.f.1 to I.f.384 which differ from the corresponding compounds of the formulae I.a.1 to I.a.384 in that A is A4 where R¹³═H and R¹⁴═CF₃:

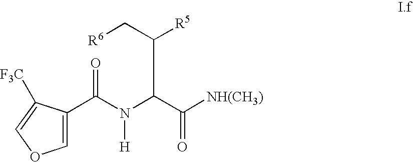

Most preference is also given to the compounds of the formula I.g, especially the compounds of the formulae I.g.1 to I.g.384 which differ from the corresponding compounds of the formulae I.a.1 to I.a.384 in that A is A5 where R¹⁴═CF₃, and R¹⁵ and R¹⁶═H:

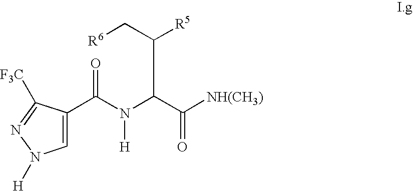

Most preference is also given to the compounds of the formula I.h, especially the compounds of the formulae I.h.1 to I.h.384 which differ from the corresponding compounds of the formulae I.a.1 to I.a.384 in that A is A5 where R¹⁴═CF₃, R¹⁵═H and R¹⁶═CH₃:

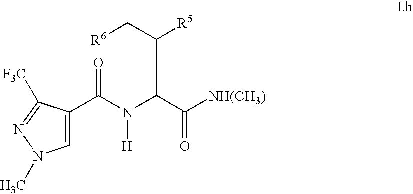

Most preference is also given to the compounds of the formula I.j, especially the compounds of the formulae I.j.1 to I.j.384 which differ from the corresponding compounds of the formulae I.a.1 to I.a.384 in that A is A8 where R¹³═H and R¹⁴═CF₃:

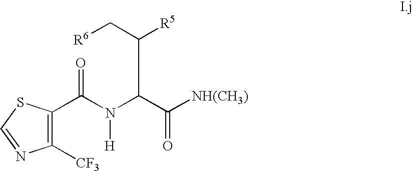

Most preference is also given to the compounds of the formula I.k, especially the compounds of the formulae I.k.1 to I.k.384 which differ from the corresponding compounds of the formulae I.a.1 to I.a.384 in that A is A8 where R¹³═CH₃ and R¹⁴═CF₃:

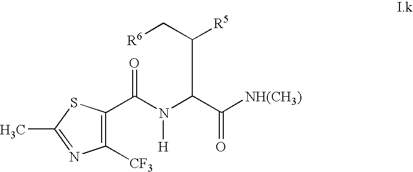

Most preference is also given to the compounds of the formula I.l, especially the compounds of the formulae I.l.1 to I.l.384 which differ from the corresponding compounds of the formulae I.a.1 to I.a.384 in that A is A10 where R¹³═CH₃ and R¹⁴═CF₃:

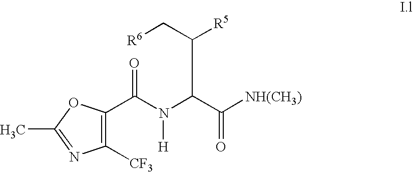

Most preference is also given to the compounds of the formula I.m, especially the compounds of the formulae I.m.1 to I.m.384 which differ from the corresponding compounds of the formulae I.a.1 to I.a.384 in that A is A11 where R¹³═CH₃ and R¹⁴═CF₃:

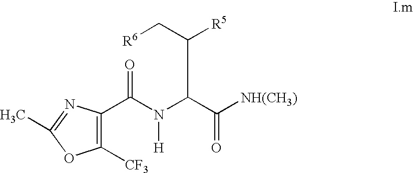

The benzoyl-substituted alanines of the formula I can be obtained by different routes, for example by the following processes:

### Process A

Alanine derivatives of the formula V are initially reacted with heteroaryl acids/heteroaryl acid derivatives of the formula IV to give the corresponding heteroaroyl derivatives of the formula III which then react with amines of the formula II to give the desired heteroaroyl-substituted alanines of the formula I:

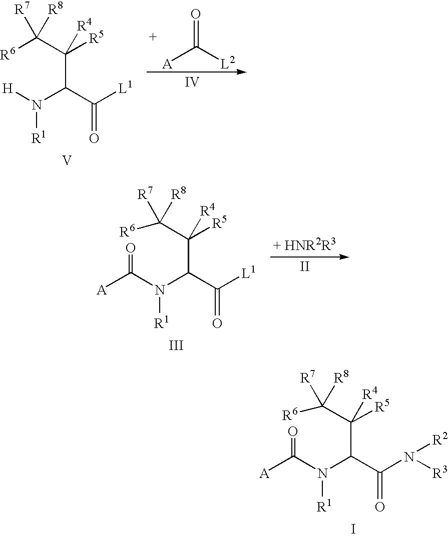

L¹ is a nucleophilically displaceable leaving group, for example hydroxyl or C₁-C₆-alkoxy.

L² is a nucleophilically displaceable leaving group, for example hydroxyl, halogen, C₁-C₆-alkylcarbonyl, C₁-C₆-alkoxycarbonyl, C₁-C₄-alkylsulfonyl, phosphoryl or isoureyl.

The reaction of the alanine derivatives of the formula V with heteroaryl acids/heteroaryl acid derivatives of the formula IV where L² is hydroxyl to give heteroaroyl derivatives of the formula III is carried out in the presence of an activating agent and a base, usually at temperatures of from 0° C. to the boiling point of the reaction mixture, preferably at from 0° C. to 110° C., particularly preferably at room temperature, in an inert organic solvent [cf. C. Montalbetti et al., Tetrahedron 2005, 61, 10827 and the literature cited therein].

Suitable activating agents are condensing agents, such as, for example, polystyrene-supported dicyclohexylcarbodiimide, diisopropylcarbodiimide, carbonyldiimidazole, chloroformates, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCl) or sulfonyl chlorides, such as methanesulfonyl chloride, toluenesulfonyl chloride or benzenesulfonyl chloride.

Suitable solvents are aliphatic hydrocarbons, such as pentane, hexane, cyclohexane and mixtures of C₅-C₈-alkanes, aromatic hydrocarbons such as benzene, toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran (THF), nitriles, such as acetonitrile and propionitrile, ketones, such as acetone, methyl ethyl ketone, diethyl ketone and tert-butyl methyl ketone, and also dimethyl sulfoxide, dimethylformamide (DMF), dimethylacetamide (DMA) and N-methylpyrrolidone (NMP) or else in water; particular preference is given to methylene chloride, THF and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methylmorpholine, and N-methylpiperidine, pyridine, substituted pyridines, such as collidine, lutidine and 4-dimethylaminopyridine, and also bicyclic amines. Particular preference is given to sodium hydroxide, triethylamine and pyridine.

The bases are generally employed in equimolar amounts. However, they can also be used in excess or, if appropriate, as solvents.

The starting materials are generally reacted with one another in equimolar amounts. It may be advantageous to employ an excess of IV, based on V.

The reaction mixtures are worked up in a customary manner, for example by mixing with water, separating the phases and, if appropriate, chromatographic purification of the crude products. Some of the intermediates and end products are obtained in the form of viscous oils which are purified or freed from volatile components under reduced pressure and at moderately elevated temperature. If the intermediates and end products are obtained as solids, purification may also be carried out by recrystallization or digestion.

The reaction of the alanine derivatives of the formula V with heteroaryl acids/heteroaryl acid derivatives of the formula IV where L² is halogen, C₁-C₆-alkylcarbonyl, C₁-C₆-alkoxycarbonyl, C₁-C₄-alkylsulfonyl, phosphoryl or isoureyl to give heteroaroyl derivatives of the formula III is carried out in the presence of a base, usually at temperatures of from 0° C. to the boiling point of the reaction mixture, preferably at from 0° C. to 100° C., particularly preferably at room temperature, in an inert organic solvent [cf. C. Montalbetti et al., Tetrahedron 2005, 61, 10827 and the literature cited therein].

Suitable solvents are aliphatic hydrocarbons, such as pentane, hexane, cyclohexane and mixtures of C₅-C₈-alkanes, aromatic hydrocarbons such as benzene, toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran (THF), nitriles, such as acetonitrile and propionitrile, ketones, such as acetone, methyl ethyl ketone, diethyl ketone and tert-butyl methyl ketone, and also dimethyl sulfoxide, dimethylformamide (DMF), dimethylacetamide (DMA) and N-methylpyrrolidone (NMP) or else in water; particular preference is given to methylene chloride, THF and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methylmorpholine, and N-methylpiperidine, pyridine, substituted pyridines, such as collidine, lutidine and 4-dimethylaminopyridine, and also bicyclic amines. Particular preference is given to sodium hydroxide, triethylamine and pyridine.

The bases are generally employed in equimolar amounts. However, they can also be used in excess or, if appropriate, as solvents.

The starting materials are generally reacted with one another in equimolar amounts. It may be advantageous to employ an excess of IV, based on V.

Work-up and isolation of the products can be carried out in a manner known per se.

It is, of course, also possible to react initially, in an analogous manner, the alanine derivatives of the formula V with amines of the formula II to give the corresponding amides which then react with heteroaryl acids/heteroaryl acid derivatives of the formula IV to give the desired heteroaroyl-substituted alanines of the formula I.

The alanine derivatives of the formula V (for example where L¹═hydroxyl or C₁-C₆-alkoxy) required for preparing the heteroaroyl derivatives of the formula III are, even in enantiomerically and diastereomerically pure form, known from the literature, or they can be prepared in accordance with the literature cited:

1. Addition of glycine enolate equivalents to nitroolefins:
B. Mendler et al., Org. Lett. 2005, 7(9), 1715; D. Dixon et al., Org. Lett. 2004, 6(24), 4427; M. Alcantara et al., Synthesis 1996, (1), 64; M. Rowley et al., Tetrahedron 1992, 48(17), 3557.
   2. Rearrangement of glycine allylamine derivatives:
J. Blid et al., J. of the Am. Chem. Soc. 2005, 27(26), 9352. H. Mues et al., Synthesis 2001, (3), 487; U. Kazmaier, Angew. Chem. 1994, 106(9), 1046.
   3. Addition of glycine enolate equivalents to epoxides:
V. Rolland-Fulcrand et al., Europ. J. of Org. Chem. 2004, (4), 873; U. Schoellkopf et al., Angew. Chem. 1986, 98(8), 755.

The heteroaryl acids/heteroaryl acid derivatives of the formula IV required for preparing the heteroaroyl derivatives of the formula III are commercially available or can be prepared analogously to procedures known from the literature [for example Chang-Ling Liu et al., J. of Fluorine Chem. (2004), 125(9), 1287-1290; Manfred Schlosser et al., Europ. J. of Org. Chem. (2002), (17), 2913-2920; Hoh-Gyu Hahn et al., Agricult. Chem. and Biotech. (English Edition) (2002), 45(1), 37-42; Jonatan O Smith et al., J. of Fluorine Chem. (1997), Vol. 1996-1997, 81(2), 123-128; Etsuji Okada et al., Heterocycles (1992), 34(4), 791-798; Aliyu B. Abubakar et al., J. of Fluorine Chem. (1991), 55(2), 189-198; J. Leroy, J. of Fluorine Chem. (1991), 53(1), 61-70; Len F. Lee et al., J. of Heterocyclic Chem. (1990), 27(2), 243-245; Len F. Lee et al., J. of Heterocyclic Chem. (1985), 22(6), 1621-1630; Jacques Leroy et al., Synthesis (1982), (4), 313-315].

The reaction of the heteroaroyl derivatives of the formula III where L¹=hydroxyl or salts thereof with amines of the formula II to give the desired heteroaroyl-substituted alanines of the formula I is carried out in the presence of an activating agent and, if appropriate, in the presence of a base, usually at temperatures of from 0° C. to the boiling point of the reaction mixture, preferably at from 0° C. to 100° C., particularly preferably at room temperature, in an inert organic solvent [cf. C. Montalbetti et al., Tetrahedron 2005, 61, 10827 and the literature cited therein].

Suitable activating agents are condensing agents, such as, for example, polystyrene-supported dicyclohexylcarbodiimide, diisopropylcarbodiimide, carbonyldiimidazole, chloroformates, such as methyl chloroformate, ethyl chloroformate, isopropyl chloroformate, isobutyl chloroformate, sec-butyl chloroformate or allyl chloroformate, pivaloyl chloride, polyphosphoric acid, propanephosphonic anhydride, bis(2-oxo-3-oxazolidinyl)-phosphoryl chloride (BOPCl) or sulfonyl chlorides, such as methanesulfonyl chloride, toluenesulfonyl chloride or benzenesulfonyl chloride.

Suitable solvents are aliphatic hydrocarbons, such as pentane, hexane, cyclohexane and mixtures of C₅-C₈-alkanes, aromatic hydrocarbons such as benzene, toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide (DMF), dimethylacetamide (DMA) and N-methylpyrrolidone (NMP) or else in water; particular preference is given to methylene chloride, THF, methanol, ethanol and water.

It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methylmorpholine, and N-methylpiperidine, pyridine, substituted pyridines, such as collidine, lutidine and 4-dimethylaminopyridine, and also bicyclic amines. Particular preference is given to sodium hydroxide, triethylamine, ethyldiisopropylamine, N-methylmorpholine and pyridine.

The bases are generally employed in catalytic amounts; however, they can also be employed in equimolar amounts, in excess or, if appropriate, as solvent.

The starting materials are generally reacted with one another in equimolar amounts. It may be advantageous to employ an excess of II, based on III.

Work-up and isolation of the products can be carried out in a manner known per se.

The reaction of the heteroaroyl derivatives of the formula III where L¹=C₁-C₆-alkoxy with amines of the formula II to give the desired heteroaroyl-substituted alanines of the formula I is usually carried out at temperatures of from 0° C. to the boiling point of the reaction mixture, preferably at from 0° C. to 10° C., particularly preferably at room temperature, in an inert organic solvent, if appropriate in the presence of a base [cf. C. Montalbetti et al., Tetrahedron 2005, 61, 10827 and the literature cited therein].

Suitable solvents are aliphatic hydrocarbons, such as pentane, hexane, cyclohexane and mixtures of C₅-C₈-alkanes, aromatic hydrocarbons such as benzene, toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide (DMF), dimethylacetamide (DMA) and N-methylpyrrolidone (NMP) or else in water; particular preference is given to methylene chloride, THF, methanol, ethanol and water.

It is also possible to use mixtures of the solvents mentioned.

The reaction can, if appropriate, be carried out in the presence of a base. Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine, N-methylmorpholine, and N-methylpiperidine, pyridine, substituted pyridines, such as collidine, lutidine and 4-dimethylaminopyridine, and also bicyclic amines. Particular preference is given to sodium hydroxide, triethylamine, ethyldiisopropylamine, N-methylmorpholine and pyridine.

The bases are generally employed in catalytic amounts; however, they can also be employed in equimolar amounts, in excess or, if appropriate, as solvent.

The starting materials are generally reacted with one another in equimolar amounts. It may be advantageous to employ an excess of II, based on III.

Work-up and isolation of the products can be carried out in a manner known per se.

The amines of the formula II required for preparing the heteroaroyl-substituted alanines of the formula I are commercially available.

### Process B

Heteroaroyl derivatives of the formula III where R⁶═NO₂ and R⁸=hydrogen can also be obtained by condensing acylated glycine derivatives of the formula VIII where the acyl group may be a removable protective group such as benzyloxycarbonyl (cf. VIIIa where Σ=benzyl) or tert-butyloxycarbonyl (cf. VIIIa where Σ=tert-butyl) with nitroolefines VII to give the corresponding addition products VI where R⁶═NO₂ and R⁸=hydrogen. The protective group is then removed, and the alanine derivative of the formula V formed in this manner where R⁶═NO₂ and R⁸=hydrogen is acylated with heteroaryl acids/heteroaryl acid derivatives of the formula IV.

Analogously, it is also possible to react an acylated glycine derivative of the formula VIII where the acyl group is a substituted heteroaroyl radical (cf. VIIIb) in the presence of a base with a nitroolefin VII to give the heteroaroyl derivative III where R⁶═NO₂ and R⁸=hydrogen:

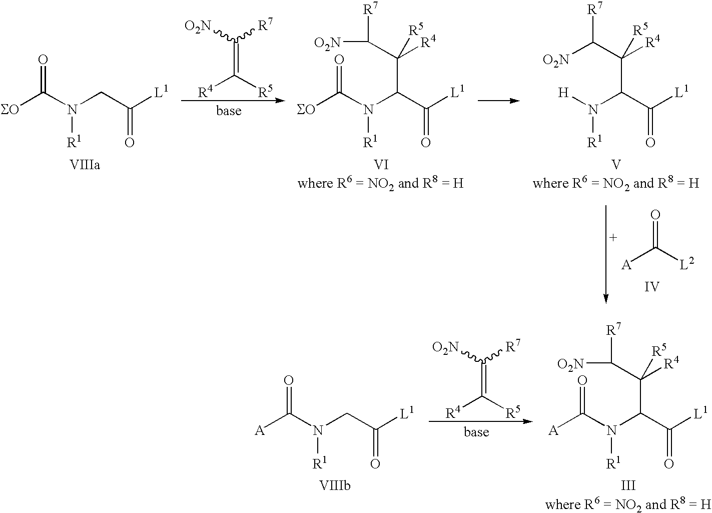

L¹ is a nucleophilically displaceable leaving group, for example hydroxyl or C₁-C₆-alkoxy.

L² is a nucleophilically displaceable leaving group, for example hydroxyl, halogen, C₁-C₆-alkylcarbonyl, C₁-C₆-alkoxycarbonyl, C₁-C₄-alkylsulfonyl, phosphoryl or isoureyl.

The reaction of the glycine derivatives VIII with nitroolefins VII to give the corresponding addition product VI where R⁶═NO₂ and R⁸=hydrogen or heteroaroyl derivative III where R⁶═NO₂ and R⁸=hydrogen is usually carried out at temperatures of from −100° C. to the boiling point of the reaction mixture, preferably from −80° C. to 20° C., especially preferably from −80° C. to −20° C., in an inert organic solvent in the presence of a base (cf. B. Mendler et al., Organic Lett. 2005, 7 (9), 1715; D. Dixon et al., Organic Lett. 2004, 6 (24), 4427; M. Alcantara et al., Synthesis 1996, (1), 64; M. Rowley et al., Tetrahedron 1992, 48 (17), 3557).

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, tert-butylmethyl ether, dioxane, anisole and tetrahydrofuran, and also dimethyl sulfoxide, dimethylformamide and dimethylacetamide, particularly preferably diethyl ether, dioxane and tetrahydrofuran. It is also possible to use mixtures of the solvents mentioned.

Suitable bases are, in general, inorganic compounds, such as alkali metal and alkaline earth metal hydrides, such as lithium hydride, sodium hydride, potassium hydride and calcium hydride, alkali metal amides, such as lithium isopropylamide and lithium hexamethyldisilazide, organometallic compounds, in particular alkali metal alkyls, such as methyllithium, butyllithium and phenyllithium, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example, tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to sodium hydride, lithium hexamethyldisilazide and lithium diisopropylamide.

The bases are generally employed in equimolar amounts; however, they can also be employed in catalytic amounts, in excess or, if appropriate, as solvent.

The starting materials are generally reacted with one another in equimolar amounts. It may be advantageous to employ an excess of the base and/or the imino compounds VII, based on the glycine derivatives VIII.

Work-up and isolation of the products can be carried out in a manner known per se.

The glycine derivatives of the formula VIII required for preparing the heteroaroyl derivatives III where R⁶═NO₂ and R⁸=hydrogen are commercially available, known from the literature [for example H. Pessoa-Mahana et al., Synth. Comm. 32, 1437 (2002)] or can be prepared in accordance with the literature cited.

The removal of the protective group Σ to give alanine derivatives of the formula V where R⁶═NO₂ and R⁸=hydrogen is carried out by methods known from the literature [cf. J.-F. Rousseau et al., J. Org. Chem. 63, 2731-2737 (1998); J. M. Andres, Tetrahedron 56, 1523 (2000)]; in the case of Σ=benzyl by hydrogenolysis, preferably using hydrogen and Pd/C in methanol; in the case of Σ=tert-butyl using acid, preferably using hydrochloric acid in dioxane.

The reaction of the alanine derivatives V where R⁶═NO₂ and R⁸=hydrogen with heteroaryl acids/heteroaryl acid derivatives IV to give heteroaroyl derivatives III where R⁶═NO₂ and R⁸=hydrogen is usually carried out analogously to the reaction, mentioned under process A, of the alanine derivatives of the formula V with heteroaryl acids/heteroaryl acid derivatives of the formula IV to give heteroaroyl derivatives III.

The heteroaroyl derivatives, obtainable in this manner, of the formula III where R⁶═NO₂ and R⁸=hydrogen can be reacted with amines of the formula II analogously to process A to give the desired heteroaroyl-substituted alanines of the formula I where R⁶═NO₂ and R⁸=hydrogen, which can then, if desired, initially be reduced to give heteroaroyl-substituted alanines of the formula I where R⁶═NH₂ and R⁸=hydrogen. The heteroaroyl-substituted alanines of the formula I where R⁶═NH₂ and R⁸=hydrogen obtained in this manner can then be derivatized with compounds IX to give heteroaroyl-substituted alanines of the formula I where R⁶═NHR¹⁰ [cf., for example, Yokokawa, F. et al., Tetrahedron Lett. 42 (34), 5903-5908 (2001); Arrault, A. et al., Tetrahedron Lett. 43(22), 4041-4044 (2002)].

It is also possible to initially reduce the heteroaroyl derivatives of the formula III where R⁶═NO₂ and R⁸=hydrogen to give further heteroaroyl derivatives of the formula III where R⁶═NH₂ and R⁸=hydrogen and then, if desired, derivatize with compounds IX to give heteroaroyl derivatives of the formula III where R⁶═NHR¹⁰ and R⁸=hydrogen [cf., for example, Jung-Hui Sun et al., Heterocycles (2004), 63(7), 585-1599; Christian Lherbet et al., Bioorg. and Med. Chem. Lett. (2003), 13(6), 997-1000; Masami Otsuka et al., Chem. and Pharm. Bull. (1985), 33(2), 509-514; J. R Piper et al., J. of Med. Chem. (1985), 28(8), 1016-1025]. The heteroaroyl derivatives of the formula III where R⁶═NHR¹³ and R⁸=hydrogen obtained in this manner can then be reacted analogously to process A with amines of the formula II to give the desired heteroaroyl-substituted alanines of the formula I where R⁶═NHR¹³ and R⁸=hydrogen:

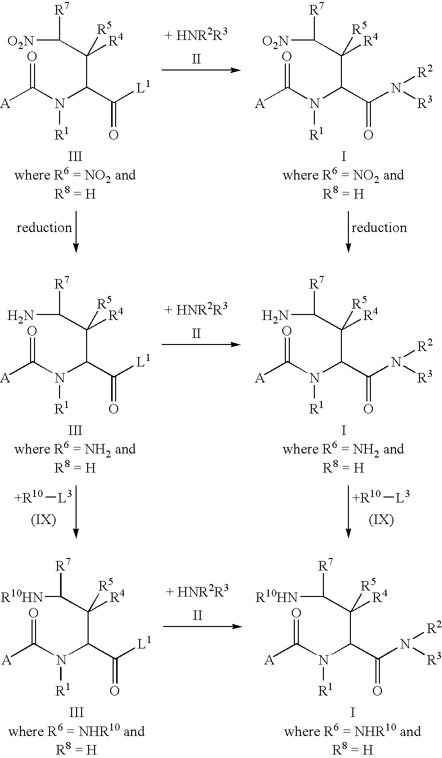

L¹ is a nucleophilically displaceable leaving group, for example hydroxyl or C₁-C₆-alkoxy.

L³ is a nucleophilically displaceable leaving group, for example halogen, hydroxyl or C₁-C₆-alkoxy.

The reaction of the heteroaroyl derivatives of the formula III where R⁶═NO₂, NH₂ or NHR¹⁰ and R⁸=hydrogen with amines of the formula II to give heteroaroyl-substituted alanines of the formula I where R⁶═NO₂, NH₂ or NHR¹⁰ and R⁸=hydrogen usually takes place analogously to the reaction, described under Process A, of the heteroaroyl derivatives of the formula III with amines of the formula II.

The reduction of the heteroaroyl derivatives of the formula III where R⁶═NO₂ and R⁸=hydrogen to give heteroaroyl derivatives of the formula III where R⁶═NH₂ and R⁸=hydrogen, and the reduction of the heteroaroyl-substituted alanines of the formula I where R⁶═NO₂ and R⁸=hydrogen to give heteroaroyl-substituted alanines of the formula I where R⁶═NH₂ and R⁸=hydrogen is usually carried out at temperatures of from 0° C. to 100° C., preferably at from 10° C. to 50° C., in an inert organic solvent in the presence of a reducing agent.

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably dichloromethane, tert-butylmethyl ether, dioxane and tetrahydrofuran. It is also possible to use mixtures of the solvents mentioned.

Suitable reducing agents are transition metal catalysts (for example Pd/C or Raney-Ni) in combination with hydrogen.

Work-up and isolation of the products can be carried out in a manner known per se.

The reduction of the nitro derivatives of the formula II or I where R⁶═NO₂ is usually carried out at a temperature of from −100° C. to the boiling point of the reaction mixture, preferably at from 0° C. to 100° C., in an inert organic solvent using a reducing agent (cf. V. Burgess et al., Aust. J. of Chem. (1988), 41(7), 1063-1070).

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, tert-butylmethyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably toluene, THF or tert-butylmethyl ether.

Suitable reducing agents are transition metal catalysts (for example Pd/C or Raney-Ni) in combination with hydrogen.

Work-up and isolation of the product can be carried out in a manner known per se.

The reaction of the heteroaroyl derivatives of the formula III where R⁶═NH₂ and R⁸=hydrogen or of the heteroaroyl-substituted alanines of the formula I where R⁶═NH₂ and R⁸=hydrogen with compounds of the formula IX to give heteroaroyl derivatives of the formula III where R⁶═NH₂ and R⁸=hydrogen or heteroaroyl-substituted alanines of the formula I where R⁶═NH₂ and R⁸=hydrogen is usually carried out at temperatures of from 0° C. to 100° C., preferably at from 10° C. to 50° C., in an inert organic solvent in the presence of a base [cf., for example, Jung-Hui Sun et al., Heterocycles (2004), 63(7), 585-1599; Christian Lherbet et al., Bioorg. and Med. Chem. Lett. (2003), 13(6), 997-1000; Masami Otsuka et al., Chem. and Pharm. Bull. (1985), 33(2), 509-514; J. R Piper et al., J. of Med. Chem. (1985), 28(8), 1016-1025].

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably dichloromethane, tert-butylmethyl ether, dioxane and tetrahydrofuran. It is also possible to use mixtures of the solvents mentioned.

Suitable bases are in general inorganic compounds such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example, tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to sodium hydroxide, sodium hydride and triethylamine.

The bases are generally employed in equimolar amounts; however, they can also be employed in catalytic amounts, in excess or, if appropriate, as solvent.

The starting materials are generally reacted with one another in equimolar amounts. It may be advantageous to employ an excess of base and/or IX, based on III or I.

Work-up and isolation of the products can be carried out in a manner known per se.

### Process C

Heteroaroyl-substituted alanines of the formula I where R¹ and R⁸=hydrogen and R⁶═OH can be obtained by converting, in a first step, glycine derivatives of the formula XII with an allyl alcohol derivative of the formula XI in the presence of a transition metal catalyst and a base, and subsequent aqueous-acidic work-up into amino derivatives which can then, in a second and third step, be acylated analogously to process A and converted into an amide X. The double bond of the amide X can then be cleaved oxidatively, and the resulting aldehyde can be reduced to heteroaroyl-substituted alanines of the formula I where R¹ and R⁸=hydrogen and R⁶═OH. The heteroaroyl-substituted alanines of the formula I where R¹ and R⁸=hydrogen and R⁶═OH obtained in this manner can for their part be derivatized into further heteroaroyl-substituted alanines of the formula I where R¹ and R⁸=hydrogen and R⁶═OR⁹, where R⁹ is not hydrogen:

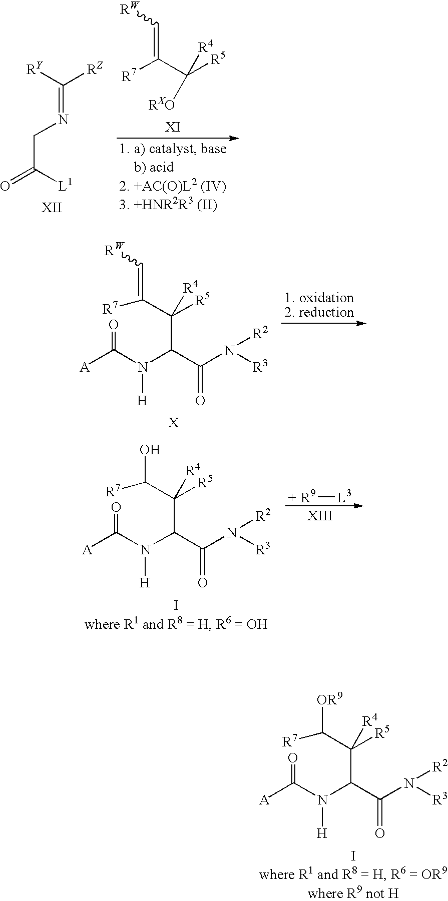

L¹ is a nucleophilically displaceable leaving group, for example hydroxyl or C₁-C₆-alkoxy.

L² is a nucleophilically displaceable leaving group, for example hydroxyl, halogen, C₁-C₆-alkylcarbonyl, C₁-C₆-alkoxycarbonyl, C₁-C₄-alkylsulfonyl, phosphoryl or isoureyl.

L³ is a nucleophilically displaceable leaving group, for example halogen, hydroxyl or C₁-C₆-alkoxy.

R^{Y} and R^{Z} are hydrogen, C₁-C₆-alkyl or aryl.

R^{W} is hydrogen or R⁵.

R^{X} is an acyl group, such as C₁-C₆-alkylcarbonyl (for example methylcarbonyl) or C₁-C₆-alkoxycarbonyl (for example methoxycarbonyl).

The reaction of the glycine derivatives of the formula XII with an allyl alcohol derivative of the formula XI is usually carried out at temperatures of from −100° C. to the boiling point of the reaction mixture, preferably from −80° C. to 80° C., especially preferably from −20° C. to 50° C., in an inert organic solvent in the presence of a transition metal catalyst and a base, followed by aqueous-acidic work-up.

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; particular preference is given to toluene, THF and acetonitrile.

It is also possible to use mixtures of the solvents mentioned.

Preferred for use as catalysts are palladium, iridium or molybdenum catalysts, preferably in the presence of a phosphine ligand, such as triphenylphosphine. In the presence of a chiral phosphine ligand, the reaction may also be carried out in an enantioselective manner (cf. D. Ikeda et al., Tetrahedron Lett. 2005, 46(39), 6663; T. Kanayama et al., J. of Org. Chem. 2003, 68(16), 6197; I. Baldwin et al., Tetrahedron Asym. 1995, 6(7), 1515; J. Genet et al., Tetrahedron 1988, 44(17), 5263).

Suitable bases are in general inorganic compounds such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate and also alkali metal bicarbonates, such as sodium bicarbonate, alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, for example, tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to carbonates, such as Na₂CO₃.

The bases are generally employed in equimolar amounts; however, they can also be employed in excess or, if appropriate, as solvent.

The subsequent steps 2 and 3 can be carried out analogously to the reaction, described under process A, of alanine derivatives of the formula V with heteroaryl acids/heteroaryl acid derivatives of the formula IV to give the corresponding heteroaroyl derivatives of the formula III and subsequent reaction of the reaction product with amines of the formula II to give the desired heteroaroyl-substituted alanines of the formula I.

The starting materials are generally reacted with one another in equimolar amounts. It may be advantageous to employ an excess of base and/or IX, based on III or I.

Work-up and isolation of the products can be carried out in a manner known per se.

The glycine derivatives of the formula XII required can be obtained analogously to methods known from the literature (cf. Vicky A. Burgess et al., Aust. J. of Chem. (1988), 41(7), 1063-1070).

The required allyl alcohol derivatives of the formula XI are commercially available.

The oxidation of the double bond to the aldehyde is usually carried out at temperatures of from −100° C. to the boiling point of the reaction mixture, preferably from −80° C. to 40° C., especially preferably from −80° C. to 0° C., in an inert organic solvent in the presence of an oxidizing agent.

Preferably, the oxidation is carried out using ozone or by sequential dihydroxylation with osmium catalysts such as OsO₄ or permanganates such as KMnO₄ and subsequent cleavage of the diol, which is preferably carried out using NalO₄ (cf. A. Siebum et al., J. Europ. J. of Org. Chem. 2004, (13), 2905; S. Hanessian et al., J. of Med. Chem. (2001), 44(19), 3074; J. Sabol et al., Tetrahedron Lett. 1997, 38(21), 3687; D. Hallett et al., J. of Chem. Soc., Chem. Comm. 1995, (6), 657).

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butylmethyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; particular preference is given to toluene, THF and acetone.

It is also possible to use mixtures of the solvents mentioned.

Work-up and isolation of the product can be carried out in a manner known per se.

The subsequent reduction to heteroaroyl-substituted alanines of the formula I where R¹ and R⁸=hydrogen and R⁶═OH is usually carried out at temperatures of from −100° C. to the boiling point of the reaction mixture, preferably from −80° C. to 40° C., especially preferably from −80° C. to 20° C., in an inert organic solvent in the presence of a reducing agent.

Preferred reducing agents are borohydrides such as NaBH₄ (cf. A. Siebum et al., J. Europ. J. of Org. Chem. 2004, (13), 2905; S. Hanessian et al., J. of Med. Chem. (2001), 44(19), 3074; J. Sabol et al., Tetrahedron Lett. 1997, 38(21), 3687; D. Hallett et al., J. of Chem. Soc., Chem. Comm. 1995, (6), 657).

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, tert-butylmethyl ether, dioxane, anisole and tetrahydrofuran (THF), alcohols, such as methanol, ethanol, n-propanol, isopropanol, n-butanol and tert-butanol, and also dimethylformamide and dimethylacetamide; particular preference is given to toluene, THF and dioxane.

It is also possible to use mixtures of the solvents mentioned.

Work-up and isolation of the product can be carried out in a manner known per se.

The derivatization of the heteroaroyl-substituted alanines of the formula I where R¹ and R⁸ and R⁶═OH with compounds of the formula XIII to give heteroaroyl-substituted alanines of the formula I where R¹ and R⁸ and R⁶═OR⁹, where R⁹ is not hydrogen, is usually carried out at temperatures of from 0° C. to 100° C., preferably from 10° C. to 50° C., in an inert organic solvent in the presence of a base [cf., for example, Jung-Hui Sun et al., Heterocycles (2004), 63(7), 585-1599; Christian Lherbet et al., Bioorg. and Med. Chem. Lett. (2003), 13(6), 997-1000; Masami Otsuka et al., Chem. and Pharm. Bull. (1985), 33(2), 509-514; J. R Piper et al., J. of Med. Chem. (1985), 28(8), 1016-1025].

Suitable solvents are aliphatic hydrocarbons, such as pentane, hexane, cyclohexane and mixtures of C₅-C₈-alkanes, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, and also dimethyl sulfoxide, dimethylformamide and dimethylacetamide, particularly preferably dichloromethane, tert-butylmethyl ether, dioxane and tetrahydrofuran. It is also possible to use mixtures of the solvents mentioned.

Suitable bases are in general inorganic compounds such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also organic bases, for example, tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to sodium carbonate, sodium hydride and triethylamine.

The bases are generally employed in equimolar amounts; however, they can also be employed in catalytic amounts, in excess or, if appropriate, as solvent.

The starting materials are generally reacted with one another in equimolar amounts. It may be advantageous to employ an excess of base and/or XIII, based on I.

Work-up and isolation of the products can be carried out in a manner known per se.

Heteroaroyl derivatives of the formula III

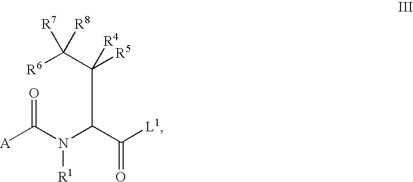

where A, R¹ and R⁴, R⁵, R⁶, R⁷ and R⁸ are as defined above and L¹ is a nucleophilically displaceable leaving group, for example hydroxyl or C₁-C₆-alkoxy, are also provided by the present invention.

The particularly preferred embodiments of the intermediates with respect to the variables correspond to those of the radicals A, R¹ and R⁴ to R⁷ of formula I.

Particular preference is given to heteroaroyl derivatives of the formula III in which
A is 5- or 6-membered heteroaryl selected from the group consisting of thienyl, furyl, pyrazolyl, imidazolyl, thiazolyl, oxazolyl and pyridyl; where the
   heteroaryl radicals mentioned may be partially or fully halogenated and/or may carry 1 to 3 radicals from the group consisting of C₁-C₆-alkyl, C₃-C₆-cycloalkyl and C₁-C₆-haloalkyl;
R¹ is hydrogen;
R⁴ is hydrogen;
R⁵ is C₂-C₆-alkenyl, C₁-C₆-haloalkyl, 3- to 6-membered heterocyclyl, C₁-C₆-alkoxy-C₁-C₄-alkyl, C₁-C₆-alkoxy-C₁-C₄-alkoxy-C₁-C₄-alkyl, aminocarbonyl, C₁-C₆-alkylcarbonylamino-C₁-C₄-alkyl, formylamino-C₁-C₄-alkyl, phenyl or heteroaryl, where the 3- to 6-membered heterocyclyl and the phenyl- and heteroaryl radicals mentioned above may be partially or fully halogenated and/or may carry one to three C₁-C₆-alkyl radicals;
R⁷ and R⁸ are hydrogen;
R⁹ and R¹⁰ are hydrogen, C₁-C₆-alkyl, formyl, C₁-C₆-alkylcarbonyl, C₁-C₆-haloalkylcarbonyl, C₁-C₆-alkoxycarbonyl, aminocarbonyl, (C₁-C₆-alkyl)aminocarbonyl, di(C₁-C₆-alkyl)aminocarbonyl, N—(C₁-C₆-alkoxy)-N—(C₁-C₆-alkyl)-aminocarbonyl, [(C₁-C₆-alkyl)aminocarbonyl(C₁-C₆-alkyl)amino]carbonyl or di(C₁-C₆-alkyl)aminothiocarbonyl or SO₂R¹²; and
R¹¹ is hydrogen.

The examples below serve to illustrate the invention.

### PREPARATION EXAMPLES

### Example 1

### 1-Methyl-N-{(1RS,2SR)-1-[(methylamino)carbonyl]-3-nitro-2-phenylpropyl}-3-(trifluoromethyl)-1H-pyrazole-4-carboxamide (Table 5, No. 5.3)

### 1.1) Ethyl ((2RS,3SR)-2-({[1-methyl-3-(trifluoromethyl)-1H-pyrazole-4-yl]carbonyl}-amino)-4-nitro-3-phenylbutyrate (Table 2, No. 2.1)

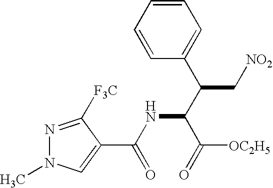

At a temperature of 0° C., 29.4 g (138 mmol) of 1-methyl-3-(trifluoromethyl)-1H-pyrazole-4-carbonyl chloride was added dropwise to a solution of 34.9 g (138 mmol) of ethyl (2RS,3SR)-2-amino-4-nitro-3-phenylbutyrate (prepared according to M. Rowley et al., Tetrahedron 1992, 48, 3557-3570) and 12.0 g (152 mmol) of pyridine in dichloromethane (400 ml). The reaction mixture obtained was stirred at room temperature for a further 16 hours, and dilute hydrochloric acid (2 M) was then added. Filtration with suction and drying of the resulting precipitate gave 8.0 g of the title compound. The organic phase of the filtrate was washed with dilute aqueous sodium hydroxide solution (2 M), dried and concentrated. By chromatographic purification (silica gel, cyclohexane/ethyl acetate), it was possible to obtain another 11.2 g of the title compound. This gave a total of 19.2 g (32.5% of theory) of the title compound.

¹H-NMR (CDCl₃): δ=1.10 (t, 3H); 3.95 (s, 3H); 3.95-4.10 (m, 3H); 4.85-4.95 (m, 2H); 5.10 (t, 1H); 6.60 (d, 1H); 7.15-7.35 (m, 5H); 7.95 (s, 1H).

### 1.2.) 1-Methyl-N-{(1RS,2SR)-1-[(methylamino)carbonyl]-3-nitro-2-phenylpropyl}-3-(trifluoromethyl)-1H-pyrazole-4-carboxamide (Table 5, No. 5.3)

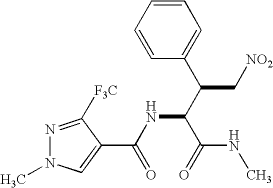

At a temperature of 0° C., methylamine was introduced into a solution of 19.2 g (44.8 mmol) of ethyl (2RS,3SR)-2-({[1-methyl-3-(trifluoromethyl)-1H-pyrazol-4-yl]carbonyl}amino)-4-nitro-3-phenylbutyrate in methanol (200 ml) until the solution was saturated. The reaction mixture obtained was stirred at room temperature for a further 16 hours. Subsequent removal of the solvent gave 18.8 g of the title compound (100% of theory; diastereomer mixture, threo/erythro >5:1) which were used without further purification for the next step.

¹H-NMR (DMSO) for the threo isomer: δ=2.35 (d, 3H); 3.95 (s, 3H); 3.90-4.05 (m, 1H); 4.75 (dd, 1H); 4.90 (dd, 1H); 5.10 (dd, 1H); 7.15-7.40 (m, 5H); 7.90 (br q, 1H); 8.50 (s, 1H); 8.55 (d, 1H).

### Example 2

### N-{(1RS,2RS)-3-Amino-1-[(methylamino)carbonyl]-2-phenylpropyl}-1-methyl-3-(trifluoromethyl)-1H-pyrazole-4-carboxamide (Table 4, No. 4.5)

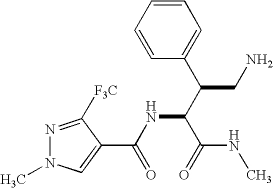

1.5 g (30.0 mmol) of hydrazine hydrate were added to a suspension von 5.0 g (12.1 mmol) of 1-methyl-N-{(1RS,2SR)-1-[(methylamino)carbonyl]-3-nitro-2-phenylpropyl}-3-(trifluoromethyl)-1H-pyrazole-4-carboxamide in methanol (100 ml). At a temperature of 40° C., about 0.5 g of Raney nickel were then added a little at a time as a methanolic suspension. The reaction mixture obtained was stirred at 40° C. for a further 2 hours. After cooling, the precipitate was filtered off and washed with methanol. The filtrate gave, after removal of the solvent, 2.7 g of the title compound (58.3% of theory, diastereomer mixture, threo/erythro >5:1) which were used without further purification for the subsequent reaction.

¹H-NMR (DMSO) for the threo isomer: δ=2.35 (d, 3H); 2.85 (d, 2H); 3.05-3.15 (m, 1H); 3.95 (s, 3H); 4.65 (d, 1H); 7.10-7.40 (m, 5H); 7.85 (br q, 1H); 8.50 (s, 1H); 8.75 (br s, 1H).

### Example 3

### 1-Methyl-N-{(1RS,2SR)-1-[(methylamino)carbonyl]-2-phenyl-3-(2,2,2-trifluoroacetylamino)propyl}-3-(trifluoromethyl)-1H-pyrazole-4-carboxamide (Table 4, No. 4.17)

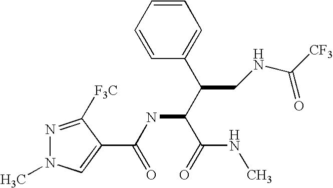

0.24 g (1.14 mmol) of trifluoroacetic anhydride was added to a solution of 0.40 g (1.04 mmol) of N-{(1RS,2RS)-3-amino-1-[(methylamino)carbonyl]-2-phenylpropyl}-1-methyl-3-(trifluoromethyl)-1H-pyrazole-4-carboxamide and 0.12 g (1.19 mmol) of triethylamine in dichloromethane (10 ml). The reaction mixture was stirred at room temperature for a further 16 hours, and dilute hydrochloric acid (2 M) was then added. The resulting precipitate was filtered off with suction and washed successively with dilute aqueous sodium hydroxide solution (2 M) and distilled water. Drying of the solid gave 0.35 g (70.2% of theory) of the title compound as a diastereomerically pure compound.

¹H-NMR (DMSO): δ=2.40 (d, 3H); 3.40-3.75 (m, 3H); 3.95 (s, 3H); 4.80 (t, 1H); 7.10-7.30 (m, 5H); 7.95 (br q, 1H); 8.45 (d, 1H); 8.50 (s, 1H); 9.20 (br t, 1H).

### Example 4

### N-(3-hydroxy-1-methylcarbamoyl-2-phenyl-propyl)-1-methyl-3-trifluoromethyl-1H-pyrazole-4-carboxamide (Table 3, No. 3.1, 3.2)

### 4.1) (E)-1-Ethoxycarbonyl-2,4-diphenylbut-3-enyl-ammonium Chloride

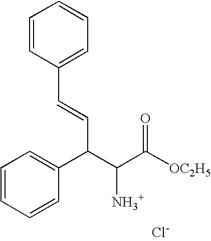

Solution A: At −60° C., 44 g (0.103 mol) of n-butyllithium (15% strength in hexane) were added dropwise to a solution of 10.4 g (0.103 mol) of diisopropylamine in 500 ml of THF. The mixture was stirred at −20° C. for 30 min and then again cooled to −60° C. A solution of 27.5 g (0.103 mol) of ethyl (benzhydrylideneamino)acetate in 30 ml of THF was added dropwise, and the mixture was stirred at −60° C. for 1 h.

Solution B: 26 g (0.103 mol) of (E)-1,3-diphenylallyl acetate (prepared according to J. Chem. Soc., Perkin Trans. 1, 2001, 2588-2594), 5.4 g (0.021 mol) of triphenylphosphine and 1.9 g (0.0052 mol) of [Pd(allyl)Cl]₂ were dissolved in 40 ml of THF.

At −60° C., solution B was added dropwise to solution A, and the mixture was stirred at this temperature for 3 h and then warmed to room temperature. Sat. NH₄Cl solution was added, and the mixture was extracted with MTBE. The combined organic phases were dried over Na₂SO₄ and the solvent was removed under reduced pressure. The residue was dissolved in 200 ml of THF, and 200 ml of hydrochloric acid (20% strength) were added. After 24 h at room temperature, the solvent was removed under reduced pressure, and twice in each case 500 ml of methanol were added to the residue and the mixture was reconcentrated under reduced pressure. The crude product was triturated with diethyl ether/CH₂Cl₂, and the solid was filtered off with suction and dried. This gave 32.8 g (96% of theory) of the title compound (diastereomer mixture) as a yellow solid of m.p. 174-176° C.

### 4.2) N-(E)-(1-Methylcarbamoyl-2,4-diphenylbut-3-enyl)-1-methyl-3-trifluoromethyl-1H-pyrazole-4-carboxamide

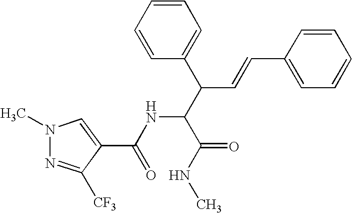

15 g (0.045 mol) of (E)-1-ethoxycarbonyl-2,4-diphenylbut-3-enylammonium chloride and 10.119 g (0.1 mol) of triethylamine were dissolved in 500 ml of CH₂Cl₂. At room temperature, 9.608 g (0.045 mol) of 1-methyl-3-trifluoromethyl-1H-pyrazole-4-carbonyl chloride were added dropwise to this solution. The mixture was stirred at room temperature for 16 h, washed with H₂O, dried over Na₂SO₄ and concentrated under reduced pressure. The residue was purified chromatographically on silica gel (CH₂Cl₂/ethyl acetate 9:1) The ethyl (E)-2-[(1-methyl-3-trifluoromethyl-1H-pyrazole-4-carbonyl)amino]-3,5-diphenylpent-4-enoate obtained (18 g, 0.038 mol) was dissolved in 190 ml of methanol, and 59.3 g (0.764 mol) of a solution of methylamine in methanol (40% strength) were added. The mixture was stirred at room temperature for 16 h, the same amount of methylamine in methanol (40% strength) was added and the mixture was stirred at room temperature for 3 d. The precipitated solid was filtered off and dried. This gave 7.4 g of the title compound (diastereomer 1) as a colorless solid of m.p. 192-194° C. The mother liquor was concentrated and stirred with diethyl ether, and the solid was filtered off and dried. This gave 8.5 g of the title compound (diastereomer 2:diastereomer 1=3:2) as a colorless solid of m.p. 239-241° C. The total yield was 15.9 g (77% of theory over two steps).

¹H-NMR (d₆-DMSO, diastereomer 1): δ=2.50 (d, 3H), 3.85 (m, 1H), 3.90 (s, 3H), 4.90 (dd, 1H), 6.35 (d, 1H), 6.50 (dd, 1H), 7.15-7.40 (m, 10H), 8.20 (m, 2H), 8.35 (d, 1H).

### 4.3) N-(3-Hydroxy-1-methylcarbamoyl-2-phenylpropyl)-1-methyl-3-trifluoromethyl-1H-pyrazole-4-carboxamide (Table 3, No. 3.1, 3.2)

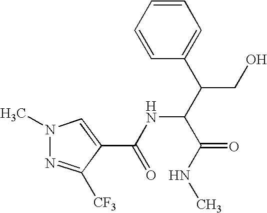

At −60° C., ozone was introduced with stirring into a solution of 6 g (13.14 mmol) of N-((E)-1-methylcarbamoyl-2,4-diphenylbut-3-enyl)-1-methyl-3-trifluoromethyl-1H-pyrazole-4-carboxamide (diastereomer ratio 3:2) in 450 ml of methanol/CH₂Cl₂ 2:1 until the solution was saturated (40 g/m³, about 50 l/h). The blueish solution was stirred at −60° C. for about another 45 min (complete conversion) and then flushed with N₂, 4.84 g (63.63 mmol) of NaBH₄ were added and the mixture was stirred at room temperature for 16 h. After filtration, the mother liquor was concentrated, and the residue was dissolved in CH₂Cl₂/THF 4:1, washed with dil. HCl, dried over Na₂SO₄ and concentrated under reduced pressure. The residue was triturated with diethyl ether and filtered off, and the solid was dried. This gave 3.1 g (61% of theory) of the title compound (diastereomer ratio 3:2) as a colorless solid.

To separate the diastereomers, acetone was added to the residue and the mixture was stirred at 50° C., and the solid was filtered off and dried. This gave 0.5 g of the title compound (diastereomer 1) as a colorless solid. The mother liquor was concentrated and triturated with methanol, and the solid was filtered off and dried. This gave 0.9 g of the title compound (diastereomer 2:diastereomer 1=6:1) as a colorless solid of m.p. 202° C. The mother liquor was reconcentrated and triturated with a little methanol, and the solid was filtered off and dried. This gave 1.1 g of the title compound (diastereomer ratio 1:1) as a colorless solid of m.p. 194° C.

¹H-NMR (d₆-DMSO, diastereomer 1): δ=2.60 (d, 3H), 3.20 (m, 1H), 3.60 (m, 2H), 3.85 (s, 3H), 4.55 (t, 1H), 4.75 (t, 1H), 7.15 (m, 1H), 7.25 (m, 4H), 8.00 (d, 1H), 8.05 (m, 1H), 8.15 (s, 1H).

¹H-NMR (d₆-DMSO, diastereomer 2): δ=2.35 (d, 3H), 3.20 (m, 1H), 3.70 (m, 1H), 3.75 (m, 1H), 3.95 (s, 3H), 4.55 (t, 1H), 4.70 (t, 1H), 7.15 (m, 1H), 7.22-7.28 (m, 4H), 7.78 (m, 1H), 8.33 (d, 1H), 8.48 (s, 1H).

### Example 5

### 3-Methylcarbamoyl-3-[(1-methyl-3-trifluoromethyl-1H-pyrazole-4-carbonyl)amino]-2-phenylpropyl Acetate (Table 3, No. 3.7)

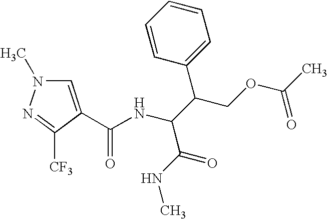

157 mg (2 mmol) of acetyl chloride were added dropwise to a solution of 400 mg (1.04 mmol) of N-(3-hydroxy-1-methylcarbamoyl-2-phenylpropyl)-1-methyl-3-trifluoromethyl-1H-pyrazole-4-carboxamide (diastereomer 1) and 202 mg (2 mmol) of triethylamine in 20 ml of CH₂Cl₂/THF 1:1, and the mixture was stirred at room temperature for 1 h. The reaction mixture was washed with H₂O and dried over Na₂SO₄, and the solvent was removed under reduced pressure. The residue was dissolved in ethyl acetate, filtered through silica gel and concentrated. The residue was triturated with diethyl ether and filtered off, and the solid was dried. This gave 200 mg (45% of theory) of the title compound as a colorless solid of m.p. 175° C. (diastereomer 1).

¹H-NMR (d₆-DMSO, diastereomer 1): δ=1.89 (s, 3H), 2.62 (d, 3H), 3.45 (m, 1H), 3.88 (s, 3H), 4.17 (dd, 1H), 4.24 (dd, 1H), 4.81 (t, 1H), 7.19 (m, 1H), 7.27 (m, 4H), 8.21 (m, 3H).

### Example 6

### 3-Methylcarbamoyl-3-[(1-methyl-3-trifluoromethyl-1H-pyrazole-4-carbonyl)amino]-2-phenylpropyl Acetate (Table 3, No. 3.8)

Analogously to the procedure above, N-(3-hydroxy-1-methylcarbamoyl-2-phenylpropyl)-1-methyl-3-trifluoromethyl-1H-pyrazole-4-carboxamide (diastereomer 2:diastereomer 1=6:1) gave the compound 3.8 (diastereomer 2:diastereomer 1=6:1) as a colorless solid of m.p. 204° C.

¹H-NMR (d₆-DMSO, diastereomer 2): δ=1.86 (s, 3H), 2.35 (d, 3H), 3.47 (m, 1H), 3.95 (s, 3H), 4.16 (dd, 1H), 4.42 (dd, 1H), 4.83 (t, 1H), 7.20 (m, 1H), 7.25-7.30 (m, 4H), 7.90 (m, 1H), 8.48 (d, 1H), 8.50 (s, 1H).

In addition to the compounds above, Tables 2 to 5 below list further heteroaroyl derivatives of the formula III and also heteroaroyl-substituted alanines of the formula I which were prepared or are preparable in a manner analogous to the processes described above.

TABLE 2

| | |
|---|---|
| | III |
| 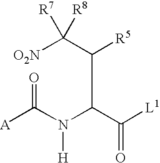 where R¹ and R⁴ = H, R⁶ = NO₂ | |

| | | | | | | Diastereomer | | |
|---|---|---|---|---|---|---|---|---|
| No. | A | R⁵ | R⁷ | R⁸ | L¹ | ration | Chirality | m.p. [° C.] |
| 2.1. | 1-CH₃-3-CF₃-4-pyrazolyl | C₆H₅ | H | H | OC₂H₅ | 5:1 | rac | 429 |
| 2.2. | 1-CH₃-3-CF₃-4-pyrazolyl | 2-CH₃-3-F—C₆H₅ | H | H | OC₂H₅ | 1:0 | rac | 461 |
| 2.3. | 1-CH₃-3-CF₃-4-pyrazolyl | 4-tetrahydropyranyl | H | H | OC₂H₅ | 1:0 | rac | 437 |

TABLE 3

| | |
|---|---|
| | I |
| 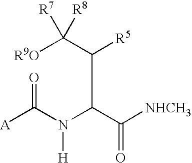 where R¹, R² and R⁴ = H, R³ = CH₃, R⁶ = OR⁹ | |

| | | | | | | Diastereomer | | m.p. | M⁺ + H |
|---|---|---|---|---|---|---|---|---|---|
| No. | A | R⁵ | R⁷ | R⁸ | R⁹ | ratio | Chirality | [° C.] | (m/z) |
| 3.1. | 1-CH₃-3-CF₃-4-pyrazolyl | C₆H₅ | H | H | H | 1:1 | rac. | 194 | |
| 3.2. | 1-CH₃-3-CF₃-4-pyrazolyl | C₆H₅ | H | H | H | 6:1 | rac. | 202 | |
| 3.3. | 1-CH₃-3-CF₃-4-pyrazolyl | C₆H₅ | H | H | CH₂COOCH₃ | 1:1 | rac. | | 457 |
| 3.4. | 1-CH₃-3-CF₃-4-pyrazolyl | C₆H₅ | H | H | (CO)NHCH₃ | 1:1 | rac. | | 442 |
| 3.5. | 1-CH₃-3-CF₃-4-pyrazolyl | C₆H₅ | H | H | (CO)N(CH₃)₂ | 1:1 | rac. | | 456 |
| 3.6. | 1-CH₃-3-CF₃-4-pyrazolyl | C₆H₅ | H | H | (CO)CH₃ | 1:1 | rac. | | 427 |
| 3.7. | 1-CH₃-3-CF₃-4-pyrazolyl | C₆H₅ | H | H | (CO)CH₃ | 0:1 | rac. | 175 | |
| 3.8. | 1-CH₃-3-CF₃-4-pyrazolyl | C₆H₅ | H | H | (CO)CH₃ | 6:1 | rac. | 194 | |
| 3.9. | 1-CH₃-3-CF₃-4-pyrazolyl | C₆H₅ | H | H | (CO)C(CH₃)₃ | 1:1 | rac. | | 469 |
| 3.10. | 1-CH₃-3-CF₃-4-pyrazolyl | C₆H₅ | H | H | (CO)CH₂COOCH₃ | 1:1 | rac. | 205 | |
| 3.11. | 1-CH₃-3-CF₃-4-pyrazolyl | C₆H₅ | H | H | (CO)CH₂CH₂COOCH₃ | 1:1 | rac. | | 499 |
| 3.12. | 1-CH₃-3-CF₃-4-pyrazolyl | C₆H₅ | H | H | (CO)CH₂CH₂CH₂COOCH₃ | 1:1 | rac. | | 513 |
| 3.13. | 1-CH₃-3-CF₃-4-pyrazolyl | C₆H₅ | H | H | (CO)CH₂CH₂CH₂CH₂COOCH₃ | 1:1 | rac. | 149 | |
| 3.14. | 1-CH₃-3-CF₃-4-pyrazolyl | C₆H₅ | H | H | (CO)N(CH₃)[(CO)NHCH₃] | 6:1 | rac. | 198 | |

TABLE 4

| | |
|---|---|
| | I |
| 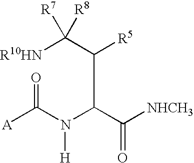 where R¹, R² and R⁴ = H, R³ = CH₃, R⁶ = NR¹⁰R¹¹ = H | |

| | | | | | | Diastereomer | | m.p. | |
|---|---|---|---|---|---|---|---|---|---|
| No. | A | R⁵ | R⁷ | R⁸ | R¹⁰ | ratio | Chirality | [° C.] | M⁺ + H (m/z) |
| 4.1. | 1-CH₃-3-CF₃-4-pyrazolyl | H | H | H | (CO)OC(CH₃)₃ | — | 2-S | 120 | |
| 4.2. | 1-CH₃-3-CF₃-4-pyrazolyl | H | H | H | (CO)N(CH₃)₂ | — | 2-S | | 379 |
| 4.3. | 1-CH₃-3-CF₃-4-pyrazolyl | H | H | H | (CO)H | — | 2-S | | 335 |
| 4.4. | 1-CH₃-3-CF₃-4-pyrazolyl | H | H | H | (CO)CH₃ | — | 2-S | | 350 |
| 4.5. | 1-CH₃-3-CF₃-4-pyrazolyl | C₆H₅ | H | H | H | >5:1 | rac. | 91 | |
| 4.6. | 1-CH₃-3-CF₃-4-pyrazolyl | C₆H₅ | H | H | (SO₂)N(CH₃)₂ | 1:0 | rac. | 210 | |
| 4.7. | 1-CH₃-3-CF₃-4-pyrazolyl | C₆H₅ | H | H | (SO₂)CH₃ | 9:1 | rac. | 245 | |
| 4.8. | 1-CH₃-3-CF₃-4-pyrazolyl | C₆H₅ | H | H | (CS)NHCH₃ | 5:1 | rac. | 193 | |
| 4.9. | 1-CH₃-3-CF₃-4-pyrazolyl | C₆H₅ | H | H | (CO)SCH₃ | 1:0 | rac. | 243 | |
| 4.10. | 1-CH₃-3-CF₃-4-pyrazolyl | C₆H₅ | H | H | (CO)SCH₂CH₃ | 1:0 | rac. | 200 | |
| 4.11. | 1-CH₃-3-CF₃-4-pyrazolyl | C₆H₅ | H | H | (CO)OCH₃ | 3:1 | rac. | 230 | |
| 4.12. | 1-CH₃-3-CF₃-4-pyrazolyl | C₆H₅ | H | H | (CO)NHCH₃ | 9:1 | rac. | 212 | |
| 4.13. | 1-CH₃-3-CF₃-4-pyrazolyl | C₆H₅ | H | H | (CO)N(CH₃)₂ | 5:1 | rac. | 171 | |
| 4.14. | 1-CH₃-3-CF₃-4-pyrazolyl | C₆H₅ | H | H | (CO)H | 5:1 | rac. | 207 | |
| 4.15. | 1-CH₃-3-CF₃-4-pyrazolyl | C₆H₅ | H | H | (CO)CH₃ | 1:0 | rac. | 155 | |
| 4.16. | 1-CH₃-3-CF₃-4-pyrazolyl | C₆H₅ | H | H | (CO)CH₂CH₃ | 1:0 | rac. | 246 | |
| 4.17. | 1-CH₃-3-CF₃-4-pyrazolyl | C₆H₅ | H | H | (CO)CF₃ | 1:0 | rac. | 233 | |
| 4.18. | 1-CH₃-3-CF₃-4-pyrazolyl | C₆H₅ | H | H | (CO)CCl₃ | 1:0 | rac. | 239 | |
| 4.19. | 1-CH₃-3-CF₃-4-pyrazolyl | 4-tetrahydropyranyl | H | H | (CO)N(CH₃)₂ | 1:0 | rac. | 242 | |
| 4.20. | 1-CH₃-3-CF₃-4-pyrazolyl | 4-tetrahydropyranyl | H | H | (CO)H | 1:0 | rac. | 231 | |
| 4.21. | 1-CH₃-3-CF₃-4-pyrazolyl | 4-tetrahydropyranyl | H | H | (CO)CH₃ | 1:0 | rac. | 298 | |
| 4.22. | 1-CH₃-3-CF₃-4-pyrazolyl | 3-F—C₆H₄ | H | H | (CO)N(CH₃)₂ | 3:1 | rac. | 168 | |
| 4.23. | 1-CH₃-3-CF₃-4-pyrazolyl | 3-F—C₆H₄ | H | H | (CO)CH₃ | 6:1 | rac. | 235 | |
| 4.24. | 1-CH₃-3-CF₃-4-pyrazolyl | 2-CH₃-3-F—C₆H₃ | H | H | (SO₂)CH₃ | 1:0 | rac. | 250 | |
| 4.25. | 1-CH₃-3-CF₃-4-pyrazolyl | 2-CH₃-3-F—C₆H₃ | H | H | (CO)OCH₃ | 1:0 | rac. | 230 | |
| 4.26. | 1-CH₃-3-CF₃-4-pyrazolyl | 2-CH₃-3-F—C₆H₃ | H | H | (CO)NHCH₃ | 1:0 | rac. | 252 | |
| 4.27. | 1-CH₃-3-CF₃-4-pyrazolyl | 2-CH₃-3-F—C₆H₃ | H | H | (CO)N(CH₃)₂ | 9:1 | rac. | 210 | |
| 4.28. | 1-CH₃-3-CF₃-4-pyrazolyl | 2-CH₃-3-F—C₆H₃ | H | H | (CO)H | 1:0 | rac. | 228 | |
| 4.29. | 1-CH₃-3-CF₃-4-pyrazolyl | 2-CH₃-3-F—C₆H₃ | H | H | (CO)CH₃ | 9:1 | rac. | 227 | |

TABLE 5

| | |
|---|---|
| | I |
| 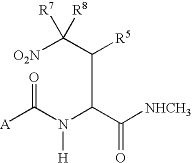 where R¹, R² and R⁴ = H, R³ = CH₃, R⁶ = NO₂ | |

| No. | A | R⁵ | R⁷ | R⁸ | Diastereomer ratio | Chirality | m.p. [° C.] |
|---|---|---|---|---|---|---|---|
| 5.1. | 1-CH₃-3-CF₃-4-pyrazolyl | 2-CH₃-3-F—C₆H₃ | H | H | 9:1 | rac. | 244 |
| 5.2. | 1-CH₃-3-CF₃-4-pyrazolyl | 2-CH₃-3-F—C₆H₃ | H | H | 1:4 | rac. | 242 |
| 5.3. | 1-CH₃-3-CF₃-4-pyrazolyl | C₆H₅ | H | H | >5:1 | rac. | 119 |
| 5.4. | 1-CH₃-3-CF₃-4-pyrazolyl | 3-F—C₆H₄ | H | H | 4:1 | rac. | 204 |
| 5.5. | 1-CH₃-3-CF₃-4-pyrazolyl | 4-tetrahydropyranyl | H | H | 1:0 | rac. | 250 |

### Biological Activity

The heteroaroyl-substituted alanines of the formula I and their agriculturally useful salts are suitable, both in the form of isomer mixtures and in the form of the pure isomers, as herbicides. The herbicidal compositions comprising compounds of the formula I control vegetation on non-crop areas very efficiently, especially at high rates of application. They act against broad-leaved weeds and grass weeds in crops such as wheat, rice, maize, soya and cotton without causing any significant damage to the crop plants. This effect is mainly observed at low rates of application.

Depending on the application method in question, the compounds of the formula I, or herbicidal compositions comprising them, can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera and Zea mays.

In addition, the compounds of the formula I may also be used in crops which tolerate the action of herbicides owing to breeding, including genetic engineering methods.

In addition, the compounds of the formula I may also be used in crops which tolerate attack by fungi or insects owing to breeding, including genetic engineering methods.

The compounds of the formula I, or the herbicidal compositions comprising them, can be used for example in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading or watering. The use forms depend on the intended purpose; in any case, they should ensure the finest possible distribution of the active compounds according to the invention.

The herbicidal compositions comprise a herbicidally effective amount of at least one compound of the formula I or an agriculturally useful salt of I, and auxiliaries which are customary for the formulation of crop protection agents.

Suitable as inert auxiliaries are essentially the following:
mineral oil fractions of medium to high boiling point, such as kerosene and diesel oil, furthermore coal tar oils and oils of vegetable or animal origin, aliphatic, cyclic and aromatic hydrocarbons, for example paraffins, tetrahydronaphthalene, alkylated naphthalenes and their derivatives, alkylated benzenes and their derivatives, alcohols such as methanol, ethanol, propanol, butanol and cyclohexanol, ketones such as cyclohexanone, strongly polar solvents, for example amines such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the substrates, either as such or dissolved in an oil or solvent, can be homogenized in water by means of a wetting agent, tackifier, dispersant or emulsifier. Alternatively, it is also possible to prepare concentrates comprising active substance, wetting agent, tackifier, dispersant or emulsifier and, if desired, solvent or oil, which are suitable for dilution with water.

Suitable surfactants (adjuvants) are the alkali metal salts, alkaline earth metal salts and ammonium salts of aromatic sulfonic acids, for example ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active compounds together with a solid carrier.

Granules, for example coated granules, impregnated granules and homogeneous granules, can be prepared by binding the active compounds to solid carriers. Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

The concentrations of the compounds of the formula I in the ready-to-use preparations can be varied within wide ranges. In general, the formulations comprise approximately from 0.001 to 98% by weight, preferably 0.01 to 95% by weight, of at least one active compound. The active compounds are employed in a purity of from 90% to 100%, preferably 95% to 100% (according to NMR spectrum).

The formulation examples below illustrate the preparation of such preparations:
I. 20 parts by weight of an active compound of the formula I are dissolved in a mixture composed of 80 parts by weight of alkylated benzene, 10 parts by weight of the adduct of from 8 to 10 mol of ethylene oxide to 1 mol of oleic acid N-monoethanolamide, 5 parts by weight of calcium dodecylbenzenesulfonate and 5 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound of the formula I.
II. 20 parts by weight of an active compound of the formula I are dissolved in a mixture composed of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound of the formula I.
III. 20 parts by weight of an active compound of the formula I are dissolved in a mixture composed of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound of the formula I.
IV. 20 parts by weight of an active compound of the formula I are mixed thoroughly with 3 parts by weight of sodium diisobutylnaphthalenesulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20 000 parts by weight of water gives a spray mixture which comprises 0.1% by weight of the active compound of the formula I.
V. 3 parts by weight of an active compound of the formula I are mixed with 97 parts by weight of finely divided kaolin. This gives a dust which comprises 3% by weight of the active compound of the formula I.
VI. 20 parts by weight of an active compound of the formula I are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.
VII. 1 part by weight of an active compound of the formula I is dissolved in a mixture composed of 70 parts by weight of cyclohexanone, 20 parts by weight of ethoxylated isooctylphenol and 10 parts by weight of ethoxylated castor oil. This gives a stable emulsion concentrate.
VIII. 1 part by weight of an active compound of the formula I is dissolved in a mixture composed of 80 parts by weight of cyclohexanone and 20 parts by weight of Wettol® EM 31 (=nonionic emulsifier based on ethoxylated castor oil). This gives a stable emulsion concentrate.

The compounds of the formula I or the herbicidal compositions can be applied pre- or post-emergence. If the active compounds are less well tolerated by certain crop plants, application techniques may be used in which the herbicidal compositions are sprayed, with the aid of the spraying equipment, in such a way that as far as possible they do not come into contact with the leaves of the sensitive crop plants, while the active compounds reach the leaves of undesirable plants growing underneath, or the bare soil surface (post-directed, lay-by).

The rates of application of the compound of the formula I are from 0.001 to 3.0, preferably 0.01 to 1.0, kg/ha of active substance (a.s.), depending on the control target, the season, the target plants and the growth stage.

To widen the spectrum of action and to achieve synergistic effects, the heteroaroyl-substituted serineamides of the formula I may be mixed with a large number of representatives of other herbicidal or growth-regulating active compound groups and then applied concomitantly. Suitable components for mixtures are, for example, 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-(het)aroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

It may furthermore be beneficial to apply the compounds of the formula I alone or in combination with other herbicides, or in the form of a mixture with other crop protection agents, for example together with agents for controlling pests or phytopathogenic fungi or bacteria. Also of interest is the miscibility with mineral salt solutions, which are employed for treating nutritional and trace element deficiencies. Non-phytotoxic oils and oil concentrates may also be added.

### Use Examples

The herbicidal activity of the heteroaroyl-substituted alanines of the formula I was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active compounds, which had been suspended or emulsified in water, were applied directly after sowing by means of finely distributing nozzles. The containers were irrigated gently to promote germination and growth and subsequently covered with transparent plastic hoods until the plants had rooted. This cover causes uniform germination of the test plants, unless this has been impaired by the active compounds.

For the post-emergence treatment, the test plants were first grown to a height of 3 to 15 cm, depending on the plant habit, and only then treated with the active compounds which had been suspended or emulsified in water. For this purpose, the test plants were either sown directly and grown in the same containers, or they were first grown separately as seedlings and transplanted into the test containers a few days prior to treatment. The rate of application for the post-emergence treatment was 1.0 kg/ha of a.s. (active substance).

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial parts, and 0 means no damage, or normal course of growth.

The plants used in the greenhouse experiments belonged to the following species:

| Scientific name | Common Name |
|---|---|
| Amaranthus retroflexus | pig weed |
| Chenopodium album | lambsquarters |
| Galium aparine | cleavers |
| Setaria viridis | green foxtail |

At application rates of 1 kg/ha, the compounds 3.1, 3.2, 3.7 and 3.14 (Table 3) and the compounds 4.8, 4.10, 4.11, 4.12, 4.13, 4.14, 4.15, 4.22, 4.23, 4.26, 4.27, 4.28 and 4.29 (Table 4) showed very good post-emergence action against the unwanted plants Amaranthus retroflexus, Chenopodium album and Setaria viridis.

Furthermore, at application rates of 1 kg/ha, the compounds 5.1 and 5.4 (Table 5) and, at application rates of 0.25 kg/ha, the compound 5.3 showed very good post-emergence action against the unwanted plants Amaranthus retroflexus, Chenopodium album and Galium aparine.

At application rates of 1 kg/ha, the compound 5.5 (Table 5) showed very good post-emergence action against the unwanted plants Amaranthus retroflexus and Chenopodium album, and good action against Galium aparine.

1-10. (canceled)

11. A heteroaroyl-substituted alanine of the formula I 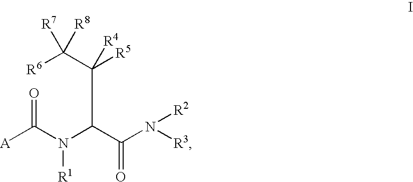
wherein
A is 5- or 6-membered heteroaryl having one to four nitrogen atoms, or having one to three nitrogen atoms and one oxygen or sulfur atom, or having one oxygen or sulfur atom, which heteroaryl may be partially or fully halogenated and/or may carry 1 to 3 radicals from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl;
R¹, R² are hydrogen, hydroxy or C₁-C₆-alkoxy;
R³ is C₁-C₆-alkyl, C₁-C₄-cyanoalkyl or C₁-C₆-haloalkyl;
R⁴ is hydrogen or C₁-C₆-alkyl;
R⁵ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₂-C₆-cyanoalkenyl, C₂-C₆-cyanoalkynyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl, or 3- to 6-membered heterocyclyl,
wherein the cycloalkyl, cycloalkenyl or 3- to 6-membered heterocyclyl radicals may be partially or fully halogenated and/or may carry one to three radicals selected from the group consisting of oxo, cyano, nitro, C₁-C₆-alkyl, C₁-C₆-haloalkyl, hydroxy, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, aminocarbonylamino, (C₁-C₆-alkylamino)carbonylamino, di(C₁-C₆-alkyl)aminocarbonylamino, aryl and aryl(C₁-C₆-alkyl); or
R⁵ is C₁-C₆-alkoxy-C₁-C₄-alkyl, C₂-C₆-alkenyloxy-C₁-C₄-alkyl, C₂-C₆-alkynyloxy-C₁-C₄-alkyl, C₁-C₆-haloalkoxy-C₁-C₄-alkyl, C₂-C₆-haloalkenyloxy-C₁-C₄-alkyl, C₂-C₆-haloalkynyloxy-C₁-C₄-alkyl, C₁-C₆-alkoxy-C₁-C₄-alkoxy-C₁-C₄-alkyl, C₁-C₆-alkylthio-C₁-C₄-alkyl, C₂-C₆-alkenylthio-C₁-C₄-alkyl, C₂-C₆-alkynylthio-C₁-C₄-alkyl, C₁-C₆-haloalkyl-C₁-C₄-thioalkyl, C₂-C₆-haloalkenyl-C₁-C₄-thioalkyl, C₂-C₆-haloalkynyl-C₁-C₄-thioalkyl, C₁-C₆-alkylsulfinyl-C₁-C₄-alkyl, C₁-C₆-haloalkylsulfinyl-C₁-C₄-alkyl, C₁-C₆-alkylsulfonyl-C₁-C₄-alkyl, C₁-C₆-haloalkylsulfonyl-C₁-C₄-alkyl, amino-C₁-C₄-alkyl, (C₁-C₆-alkyl)amino-C₁-C₄-alkyl, di(C₁-C₆-alkyl)amino-C₁-C₄-alkyl, (C₁-C₆-alkylsulfonyl)amino-C₁-C₄-alkyl, C₁-C₆-alkylsulfonyl(C₁-C₆-alkyl)amino-C₁-C₄-alkyl, C₁-C₆-alkylcarbonyl, hydroxycarbonyl, C₁-C₆-alkoxycarbonyl, aminocarbonyl, (C₁-C₆-alkyl)aminocarbonyl, di(C₁-C₆-alkyl)aminocarbonyl, formylamino-C₁-C₄-alkyl, (C₁-C₆-alkoxycarbonyl)amino-C₁-C₄-alkyl, C₁-C₆-alkylcarbonyl-C₁-C₆-alkyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, aminocarbonyl-C₁-C₄-alkyl, (C₁-C₆-alkyl)aminocarbonyl-C₁-C₄-alkyl, di(C₁-C₆-alkyl)aminocarbonyl-C₁-C₄-alkyl, (C₁-C₆-alkylcarbonyl)amino-C₁-C₄-alkyl, C₁-C₆-alkylcarbonyl(C₁-C₆-alkylamino)-C₁-C₄-alkyl, (C₁-C₆-alkyl)aminocarbonyloxy-C₁-C₄-alkyl, di(C₁-C₆-alkyl)aminocarbonyloxy-C₁-C₄-alkyl, (C₁-C₆-alkyl)aminocarbonylamino-C₁-C₄-alkyl, di(C₁-C₆-alkyl)aminocarbonylamino-C₁-C₄-alkyl, phenyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₂-C₄-haloalkynyl, phenyl-C₁-C₄-hydroxyalkyl, phenyl-C₂-C₄-hydroxyalkenyl, phenyl-C₂-C₄-hydroxyalkynyl, phenylcarbonyl-C₁-C₄-alkyl, phenylcarbonyloxy-C₁-C₄-alkyl, phenyloxycarbonyl-C₁-C₄-alkyl, phenyloxy-C₁-C₄-alkyl, phenylthio-C₁-C₄-alkyl, phenylsulfinyl-C₁-C₄-alkyl, phenylsulfonyl-C₁-C₄-alkyl,
heteroaryl, heteroaryl-C₁-C₄-alkyl, heteroaryl-C₂-C₄-alkenyl, heteroaryl-C₂-C₄-alkynyl, heteroaryl-C₁-C₄-haloalkyl, heteroaryl-C₂-C₄-haloalkenyl, heteroaryl-C₂-C₄-haloalkynyl, heteroaryl-C₁-C₄-hydroxyalkyl, heteroaryl-C₂-C₄-hydroxyalkenyl, heteroaryl-C₂-C₄-hydroxyalkynyl, heteroarylcarbonyl-C₁-C₄-alkyl, heteroarylcarbonyloxy-C₁-C₄-alkyl, heteroaryloxycarbonyl-C₁-C₄-alkyl, heteroaryloxy-C₁-C₄-alkyl, heteroarylthio-C₁-C₄-alkyl, heteroarylsulfinyl-C₁-C₄-alkyl, or heteroarylsulfonyl-C₁-C₄-alkyl,
wherein the phenyl and heteroaryl radicals may be partially or fully halogenated and/or may carry one to three radicals selected from the group consisting of cyano, nitro, C₁-C₆-alkyl, C₁-C₆-haloalkyl, hydroxy, C₁-C₆-hydroxyalkyl, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkoxycarbonyl-C₁-C₆-alkoxy, amino, C₁-C₆-alkylamino, di(C₁-C₆-alkyl)amino, C₁-C₆-alkylsulfonylamino, C₁-C₆-haloalkylsulfonylamino, (C₁-C₆-alkyl)aminocarbonylamino, di(C₁-C₆-alkyl)-aminocarbonylamino, aryl and aryl(C₁-C₆-alkyl);
R⁶ is OR⁹, NR¹⁰R¹¹ or NO₂;
R⁷ is hydrogen, C₁-C₆-alkyl or C₁-C₆-haloalkyl;
R⁸ is hydrogen, C₁-C₆-alkyl or C₁-C₆-haloalkyl;
R⁹ and R¹⁰ are independently of one another hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, formyl, C₁-C₆-alkylcarbonyl, C₁-C₆-alkylthiocarbonyl, C₃-C₆-cycloalkylcarbonyl, C₂-C₆-alkenylcarbonyl, C₂-C₆-alkynylcarbonyl, C₁-C₆-alkoxycarbonyl, C₃-C₆-alkenyloxycarbonyl, C₃-C₆-alkynyloxycarbonyl, aminocarbonyl, C₁-C₆-alkylaminocarbonyl, C₃-C₆-alkenylaminocarbonyl, C₃-C₆-alkynylaminocarbonyl, C₁-C₆-alkylsulfonylaminocarbonyl, di(C₁-C₆-alkyl)aminocarbonyl, N—(C₃-C₆-alkenyl)-N—(C₁-C₆-alkyl)aminocarbonyl, N—(C₃-C₆-alkynyl)-N—(C₁-C₆-alkyl)aminocarbonyl, N—(C₁-C₆-alkoxy)-N—(C₁-C₆-alkyl)aminocarbonyl, N—(C₃-C₆-alkenyl)-N—(C₁-C₆-alkoxy)aminocarbonyl, N—(C₃-C₆-alkynyl)-N—(C₁-C₆-alkoxy)aminocarbonyl, [(C₁-C₆-alkyl)aminocarbonyl(C₁-C₆-alkyl)amino]carbonyl, (C₁-C₆-alkyl)aminothiocarbonyl, di(C₁-C₆-alkyl)aminothiocarbonyl, (C₁-C₆-alkyl)cyanoimino, (amino)cyanoimino, (C₁-C₆-alkyl)aminocyanoimino, di(C₁-C₆-alkyl)aminocyanoimino, C₁-C₆-alkylcarbonyl-C₁-C₆-alkyl, C₁-C₆-alkoxyimino-C₁-C₆-alkyl, N—(C₁-C₆-alkylamino)imino-C₁-C₆-alkyl, N-(di-C₁-C₆-alkylamino)imino-C₁-C₆-alkyl or tri-C₁-C₄-alkylsilyl,
wherein the alkyl, cycloalkyl and alkoxy radicals mentioned may be partially or fully halogenated and/or may carry one to three groups selected from the group consisting of: cyano, hydroxy, C₃-C₆-cycloalkyl, C₁-C₆-alkoxy-C₁-C₄-alkyl, C₁-C₄-alkoxy-C₁-C₄-alkoxy-C₁-C₄-alkyl, C₁-C₄-alkoxy, C₁-C₄-alkylthio, di(C₁-C₄-alkyl)amino, C₁-C₄-alkyl-C₁-C₆-alkoxycarbonylamino, C₁-C₄-alkylcarbonyl, hydroxycarbonyl, C₁-C₄-alkoxycarbonyl, aminocarbonyl, C₁-C₄-alkylaminocarbonyl, di(C₁-C₄-alkyl)aminocarbonyl and C₁-C₄-alkylcarbonyloxy; or
R⁹ and R¹⁰ are independently of one another phenyl, phenyl-C₁-C₆-alkyl, phenylcarbonyl-C₁-C₆-alkyl, phenoxycarbonyl, phenylaminocarbonyl, phenylsulfonylaminocarbonyl, N—(C₁-C₆-alkyl)-N-(phenyl)aminocarbonyl, phenyl-C₁-C₆-alkylcarbonyl, or SO₂R¹²;
wherein the phenyl radical may be partially or fully halogenated and/or may carry one to three groups selected from the group consisting of nitro, cyano, C₁-C₄-alkyl, C₁-C₄-haloalkyl, C₁-C₄-alkoxy and C₁-C₄-haloalkoxy;
R¹¹ is hydrogen, C₁-C₆-alkyl, C₃-C₆-cycloalkyl, C₃-C₆-alkenyl, C₃-C₆-alkynyl, C₃-C₆-haloalkenyl, C₃-C₆-haloalkynyl, hydroxy or C₁-C₆-alkoxy;
R¹² is C₁-C₆-alkyl, C₁-C₆-haloalkyl, di(C₁-C₆-alkyl)amino or phenyl,
wherein the phenyl radical may be partially or fully halogenated and/or may carry one to three groups selected from the group consisting of C₁-C₆-alkyl, C₁-C₆-haloalkyl and C₁-C₆-alkoxy;
or an agriculturally useful salt thereof.

12. The heteroaroyl-substituted alanine of claim 11 where A is 5- or 6-membered heteroaryl selected from the group consisting of pyrrolyl, thienyl, furyl, pyrazolyl, imidazolyl, thiazolyl, oxazolyl, tetrazolyl, pyridyl and pyrimidinyl;
wherein the heteroaryl radicals may be partially or fully halogenated and/or may carry 1 to 3 radicals selected from the group consisting of cyano, C₁-C₆-alkyl, C₃-C₆-cycloalkyl, C₁-C₆-haloalkyl, C₁-C₆-alkoxy, C₁-C₆-haloalkoxy and C₁-C₆-alkoxy-C₁-C₄-alkyl.

13. The heteroaroyl-substituted alanine of claim 11 where R¹, R², R⁴, R⁷ and R⁸ are hydrogen.

14. The heteroaroyl-substituted alanine of claim 11 where R⁵ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-cyanoalkyl, C₁-C₆-hydroxyalkyl, C₂-C₆-hydroxyalkenyl, C₂-C₆-hydroxyalkynyl, C₃-C₆-cycloalkyl, C₃-C₆-cycloalkenyl or 3- to 6-membered heterocyclyl,
wherein the cycloalkyl, cycloalkenyl or 3- to 6-membered heterocyclyl radicals may be partially or fully halogenated and/or may carry one to three radicals selected from the group consisting of oxo, C₁-C₆-alkyl, C₁-C₆-haloalkyl, hydroxycarbonyl and C₁-C₆-alkoxycarbonyl; or
R⁵ is C₁-C₆-alkoxy-C₁-C₄-alkyl, C₁-C₆-haloalkoxy-C₁-C₄-alkyl, C₁-C₆-alkoxy-C₁-C₄-alkoxy-C₁-C₄-alkyl, C₁-C₆-alkylthio-C₁-C₄-alkyl, C₁-C₆-alkylsulfonylamino-C₁-C₄-alkyl, hydroxycarbonyl, C₁-C₆-alkoxycarbonyl, aminocarbonyl, hydroxycarbonyl-C₁-C₄-alkyl, C₁-C₆-alkoxycarbonyl-C₁-C₄-alkyl, C₁-C₆-haloalkoxycarbonyl-C₁-C₄-alkyl, C₁-C₆-alkylcarbonyloxy-C₁-C₄-alkyl, C₁-C₆-alkylcarbonylamino-C₁-C₄-alkyl, (C₁-C₆-alkyl)aminocarbonylamino-C₁-C₄-alkyl, di(C₁-C₆-alkyl)aminocarbonylamino-C₁-C₄-alkyl, di(C₁-C₆-alkyl)aminocarbonyloxy-C₁-C₄-alkyl, formylamino-C₁-C₄-alkyl, phenyl, phenyl-C₁-C₄-alkyl, phenyl-C₂-C₄-alkenyl, phenyl-C₂-C₄-alkynyl, phenyl-C₁-C₄-haloalkyl, phenyl-C₂-C₄-haloalkenyl, phenyl-C₁-C₄-hydroxyalkyl, phenyloxy-C₁-C₄-alkyl, phenylthio-C₁-C₄-alkyl, phenylsulfinyl-C₁-C₄-alkyl, phenylsulfonyl-C₁-C₄-alkyl, heteroaryl, heteroaryl-C₁-C₄-alkyl, heteroaryl-C₁-C₄-hydroxyalkyl, heteroaryloxy-C₁-C₄-alkyl, heteroarylthio-C₁-C₄-alkyl, heteroarylsulfinyl-C₁-C₄-alkyl or heteroarylsulfonyl-C₁-C₄-alkyl,
wherein the phenyl and the heteroaryl radicals may be partially or fully halogenated and/or may carry one to three radicals selected from the group consisting of cyano, nitro, C₁-C₆-alkyl, C₁-C₆-haloalkyl, hydroxy, C₁-C₆-alkoxy, C₁-C₆-haloalkoxy, hydroxycarbonyl, C₁-C₆-alkoxycarbonyl, hydroxycarbonyl-C₁-C₆-alkoxy, C₁-C₆-alkylsulfonylamino and C₁-C₆-haloalkylsulfonylamino.

15. A process for preparing the heteroaroyl-substituted alanine of claim 11, wherein an alanine derivative of the formula V 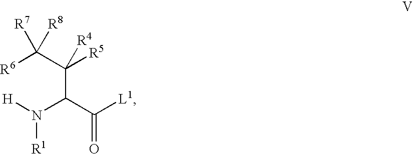
wherein R¹, R⁴, R⁵, R⁶, R⁷ and R⁸ are as defined in claim 11 and L¹ is hydroxy or C₁-C₆-alkoxy,
is reacted with a heteroaryl acid derivative of the formula IV 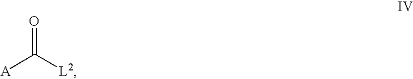
wherein A is as defined in claim 11 and L² is hydroxy, halogen, C₁-C₆-alkylcarbonyl, C₁-C₆-alkoxycarbonyl, C₁-C₄-alkylsulfonyl, phosphoryl or isoureyl,
to give the corresponding heteroaroyl derivative of the formula III 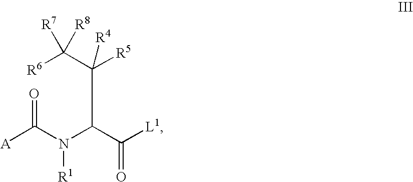
wherein A, R¹, R⁴, R⁵, R⁶ and R⁷ are as defined in claim 11 and L¹ is hydroxy or C₁-C₆-alkoxy,
and the resulting heteroaroyl derivative of the formula III is then reacted with an amine of the formula II
HNR²R³ II,
where R² and R³ are as defined in claim 11
to afford the heteroaroyl-substituted alanine of claim 11.

16. A heteroaroyl derivative of the formula III 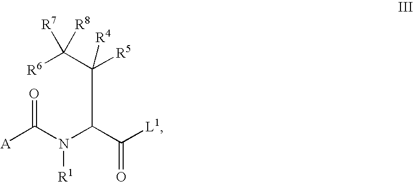
where A, R¹, R⁴, R⁵, R⁶ and R⁷ are as defined in claim 11 and L¹ is hydroxy or C₁-C₆-alkoxy.

17. A composition, comprising a herbicidally effective amount of at least one heteroaroyl-substituted alanine of claim 11 and auxiliaries customary for formulating crop protection agents.

18. A process for preparing the composition of claim 17, comprising mixing a herbicidally effective amount of the at least one heteroaroyl-substituted alanine and auxiliaries customary for formulating crop protection agents.

19. A process for preparing a composition comprising a herbicidally effective amount of at least one heteroaroyl-substituted alanine of claim 12 comprising mixing said at least one heteroaroyl-substituted alanine and auxiliaries customary for formulating crop protection agents.

20. A process for preparing a composition comprising a herbicidally effective amount of at least one heteroaroyl-substituted alanine of claim 13 comprising mixing said at least one heteroaroyl-substituted alanine and auxiliaries customary for formulating crop protection agents.

21. A process for preparing a composition comprising a herbicidally effective amount of at least one heteroaroyl-substituted alanine of claim 14 comprising mixing said at least one heteroaroyl-substituted alanine and auxiliaries customary for formulating crop protection agents.

22. A method for controlling unwanted vegetation, comprising allowing a herbicidally effective amount of at least one heteroaroyl-substituted alanine of claim 11 to act on plants, their habitat and/or on seed.

23. A method for controlling unwanted vegetation, comprising allowing a herbicidally effective amount of at least one heteroaroyl-substituted alanine of claim 12 to act on plants, their habitat and/or on seed.

24. A method for controlling unwanted vegetation, comprising allowing a herbicidally effective amount of at least one heteroaroyl-substituted alanine of claim 13 to act on plants, their habitat and/or on seed.

25. A method for controlling unwanted vegetation, comprising allowing a herbicidally effective amount of at least one heteroaroyl-substituted alanine of claim 14 to act on plants, their habitat and/or on seed.
